(19) United States
(12) Patent Application Publication
(10) Pub. No.: US 2011/0054156 A1
(43) Pub. Date: Mar. 3, 2011

(54) SYNTHESIS OF TIMOSAPONIN BII

(75) Inventors: Shuihong Cheng, Beijing (CN); Yuguo Du, Beijing (CN); Baiping Ma, Beijing (CN); Lu Li, Beijing (CN); Yang Zhao, Beijing (CN)

(73) Assignee: INSTITUTE OF RADIATION MEDICINE, ACADEMY OF MILITARY SCIENCES, PLA, Beijing (CN)

(21) Appl. No.: 12/990,336

(22) PCT Filed: Apr. 30, 2008
(86) PCT No.: PCT/CN08/00889

§ 371 Date: Oct. 29, 2010

(51) Int. Cl. C07H 17/04

(52) U.S. Cl. 536/6

(57) ABSTRACT

The invention provides a synthetic route from sarsasapogenin to timosaponin BII and related compounds. A diketone intermediate is provided, which can advantageously be used for in situ assembly of complex sugar moieties of the desired glycone end product. The diketone compound is then selectively reduced using a borohydride reducing agent to form the desired end product, certain of the end products and intermediates are novel compounds perse.

### FIELD OF THE INVENTION

The present invention relates to synthesis of timosaponin BII and related compounds.

### BACKGROUND OF THE INVENTION

Timosaponin BII, also called prototimosaponin AIII, (25S)-26-O-β-D-glucopyranosyl-22-hydroxy-5β-furostane-3β,26-diol-3-O-β-D-glucopyranosyl-(1→2)-β-D-galactopyranoside, having the formula:

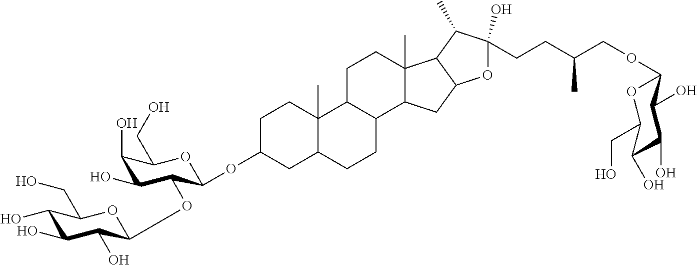

has been reported as having useful pharmaceutical properties, including activities against dementia and stroke, and abilities to lower blood sugar levels, inhibit platelet aggregation and clear free radicals. See WO-A-99/16786 (EP-A-1024146), WO-A-2005/105108 and WO-A-2005/105824 and the publications referred to therein, the disclosures of all of which are incorporated herein by reference.

Hitherto, the active agent has been extracted from plant sources, particularly the rhizomes of Anemarrhena asphodeloides Bge. The relatively small amounts of the compound available in this way limits the potential commercial development of the compound and its physiologically active analogues and derivatives.

The present invention is based on our surprising finding of a synthetic route from the more readily available material sarsasapogenin, providing timosaponin BII in good yield with an economically acceptable number of reaction steps and without the need for excessive intermediate separation or purification steps.

The sarsasapogenin is available with an acceptable purity via known processes, for example as described in WO-A-2004/037845 and WO-A-2006/048665, the disclosures of which are incorporated herein by reference.

The finding of a new synthetic route to timosaponin BII, according to the present invention, opens up the possibilities to develop novel physiologically active analogues and derivatives of timosaponin BII. The present invention thus extends also to such novel analogues and derivatives.

### BRIEF DESCRIPTION OF THE INVENTION

In a first aspect, the present invention provides a method of preparing a compound of general formula I:

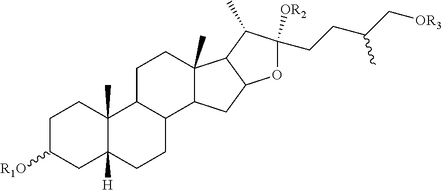

wherein, independently of each other,
R₁ represents hydrogen or an ester, ether or sugar residue;
R₂ represents hydrogen; and
R₃ represents hydrogen or a sugar residue;
or a protected form thereof in which any one or more of the groups R₁, and R₃ are, independently from each other, protected by a removable protecting group to prevent an undesirable reaction of the group;
which comprises selectively reducing a diketone compound of general formula II:

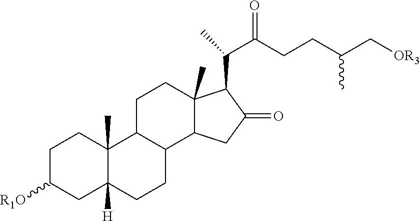

wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group to prevent an undesirable reaction of the residue;
using a borohydride reducing agent in a suitable solvent.

The expression “protected” used herein in the context of any residue will, unless the context otherwise requires, be taken to refer to the use of a removable protecting group to prevent an undesirable reaction of the residue. The expression “protecting group” and like expressions will be understood correspondingly.

Where the stereochemistry of a bond or carbon centre is defined, this is shown using the solid-wedge-bond and dotted-wedge-bond convention, a solid wedge representing a bond up (β) from the plane of the paper and a dotted wedge representing a bond down (α) from the plane of the paper. Bonds that are stereochemically undefined are shown using an unwedged bond (—) or a wavy line bond (˜). Bonds within the steroidal fused ring system are shown stereochemically undefined and any definition of their stereochemistry or stereochemistry options is to be understood from knowledge of the molecules under consideration.

The following abbreviations for sugars are used herein: Gal (galactose), Glc (glucose), Rha (rhamnose), Fuc (fucose), Xyl (xylose), Ara (arabinose).

Preferred starting materials for the method defined above are compounds of general formula IIa:

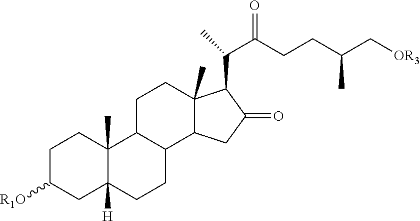

wherein, independently of each other,
R₁ represents hydrogen or an ester, ether or sugar residue; and

R₃ represents hydrogen or a sugar residue;

or a protected form thereof in which any one or both of the groups R₁ and R₃ are, independently from each other, protected by a removable protecting group to prevent an undesirable reaction of the residue.

Most preferred starting materials for the method defined above are compounds of general formula IIb:

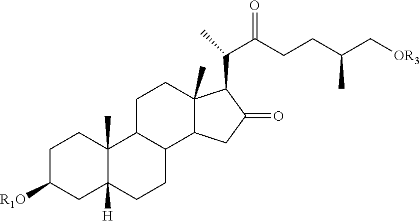

wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group to prevent an undesirable reaction of the residue.

It is especially preferred that the method is used in the preparation of timosaponin BII or a prodrug form thereof.

Protecting groups, if present, may if desired be simultaneously or subsequently removed in conventional manner. The use and removal of appropriate protecting groups will be well within the capacity of those skilled in this art.

The diketone function of the compound of general formula II has been found to be substantially inert to reaction with esterifying, etherifying and glycosylating agents for adjusting the residues R₁ and R₃. The diketone compound of formula II thus provides a suitable intermediate species for coupling reactions for adding optionally protected ester, ether and/or sugar moieties to develop the residues R₁ and R₃.

The compound of general formula I wherein R₁ represents hydrogen or an ester, ether or sugar residue, R₁ represents hydrogen and R₃ represents hydrogen or a sugar residue, or a protected form thereof, prepared as defined above, may if desired be subjected to a reaction to introduce an ester or ether residue in place of the hydrogen for R₂. Such reactions to convert an OH residue to an O-ester or O-ether residue are well known to those skilled in this art. In some cases it may be desirable to use an activated form of the compound of general formula I, for example a form of the compound in which the OH group OR₂ is converted to an O⁻A⁺ salt, wherein A⁺ is a cation, e.g. sodium. In some cases it may be desirable to use an activated form of a reagent for introducing an ester or ether residue, for example a halide form to assist a substitution reaction to introduce the residue. The selection of such features is well known to those skilled in this art.

The method of the invention can be performed on any scale from laboratory through pilot plant to commercial (kilogram) scale preparing quantities of product in excess of lkg per batch.

The above method makes available compounds of general formula I and protected forms thereof which are per se novel compounds, and they therefore constitute a further aspect of the present invention. In accordance with a second aspect of the present invention, therefore, there are provided compounds of general formula I:

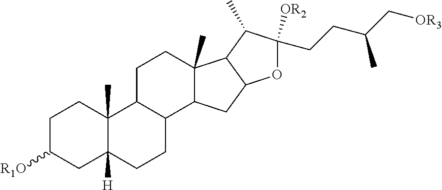

wherein, independently of each other,
R₁ denotes H, COCH₃, CO(CH₂)ₙCH₃ (n=1-6), CₘH₂ₘ₊₁ (m=1-6), Gal, Glc,

### β-D-Glc-(1→2)-β-D-Gal, β-D-Glc-(1→4)-β-D-Gal, β-D-Glc-(1→2)-β-D-Glc,

### α-D-Glc-(1→4)-β-D-Glc, β-D-Glc-(1→4)-β-D-Glc-(1→2)-β-D-Glc,

### β-L-Rha-(1→4)-β-D-Glc, α-L-Rha-(1→6)-β-D-Gal,

### α-L-Rha-(1→4)-[α-L-Rha-(1→2)]-β-D-Glc, β-D-Glc-(1→4)-[α-L-Rha-(1→2)]-β-D-Glc,

### β-D-Glc-(1→2)-[α-L-Rha-(1→4)]-β-D-Glc, β-D-Glc-(1→4)-[α-L-Rha-(1→2)]-β-D-Gal,

### α-L-Rha-(1→4)-[β-DGlc-(1→2)]-β-D-Glc, β-D-Glc-(1→4)-β-D-Glc-(1→4)-β-D-Gal,

### β-D-Glc-(1→4)-β-D-Glc-(1→2)-β-D-Gal, β-D-Glc-(1→4)-β-D-Glc-(1→4)-β-D-Glc or

### β-D-Glc-(1→4)-β-D-Glc-(1→2)-β-D-Glc;

R₂ denotes H or CₘH₂ₘ₊₁ (m=1-6); and
R₃ denotes H, α-L-Fuc, β-D-Xyl, β-D-Ara, α-L-Rha, and β-D-Glc;
excluding the compounds disclosed in WO-A-99/16786, WO-A-2005/105108 and WO-A-2005/105824 and the publications referred to therein.

In one particular embodiment of the second aspect of the present invention, the compounds are prepared using the method of the first aspect of the invention.

The intermediate compounds of general formula II, used in the method of the present invention, are also per se novel compounds, and they therefore constitute a further aspect of the present invention.

In accordance with a third aspect of the present invention, therefore, there are provided compounds of general formula

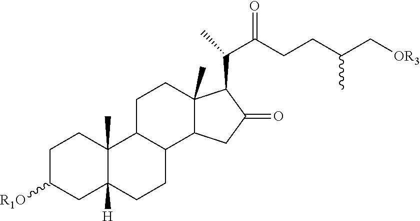

wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group.

Preferred compounds are those of general formulae IIa and IIb as defined above.

Most preferred compounds are those of general formula IIb wherein, independently of each other,

R₁ denotes H, COCH₃, CO(CH₂)ₙCH₃ (n=1-6), CₘH₂ₘ₊₁ (m=1-6), Gal, Glc,

### β-D-Glc-(1→2)-β-D-Glc, β-D-Glc-(1→4)-β-D-Gal, β-D-Glc-(1→2)-β-D-Glc,

### β-D-Glc-(1→4)-β-D-Glc, β-D-Glc-(1→6)-β-D-Glc, α-L-Rha-(1→2)-β-D-Glc,

### α-L-Rha-(1→4)-β-D-Glc, α-L-Rha-(1→6)-β-D-Gal,

### α-L-Rha-(1→4)-[α-L-Rha-(1→2)]-β-D-Glc, β-D-Glc-(1→4)-[α-L-Rha-(1→2)]-β-D-Glc,

### β-D-Glc-(1→2)-[α-L-Rha-(1→4)]-β-D-Glc, β-D-Glc-(1→4)-[α-L-Rha-(1→2)]-β-D-Gal,

### α-L-Rha-(1→4)-[β-DGlc-(1→2)]-β-D-Glc, β-D-Glc-(1→4)-β-D-Glc-(1→4)-β-D-Gal,

### β-D-Glc-(1→4)-β-D-Glc-(1→2)-β-D-Gal, β-D-Glc-(1→4)-β-D-Glc-(1→4)-β-D-Glc or

### β-D-Glc-(1→4)-β-D-Glc-(1→2)-β-D-Glc; and

R₃ denotes H, α-L-Fuc, β-D-Xyl, α-L-Rha, β-D-Gal and β-D-Glc.

### DETAILED DESCRIPTION OF THE INVENTION

### Selective Reduction of the Diketone of General Formula II

The preferred reducing agent is an unhindered borohydride reducing agent. The expression “unhindered” used herein refers to an absence of organic substituents of the borohydride species.

A suitable borohydride reducing agent is sodium borohydride.

The solvent for the selective reduction of the diketome of general formula II is selected according to the particular reducing agent used. The solvent is preferably a mixture of a polar organic solvent, such as, for example, an alkyl alcohol having more than one carbon atom, and a non-polar organic solvent which is miscible with the polar solvent, such as, for example, a haloalkane, e.g. dichloromethane. The relative proportions of the polar and non-polar solvents can be selected by a person skilled in the art according to the other compounds to be used. The polar solvent may conveniently be present in a volume excess, for example in a ratio polar:non-polar in the range of about 2:1 to about 20:1, for example about 4:1 to about 10:1 by volume. The solvent is preferably non-aqueous.

Protecting groups for R₁ and R₃ in the diketone of general formula II may be selected from conventional protecting groups according to the reaction conditions and the nature of the residue to be protected. For details of protecting groups that may be used, and the removal reactions for them, see T. W. Green, P. G. M. Wuts, “Protective Groups in Organic Synthesis”, 4^{th} Edition, Wiley-Interscience, New York, 2006, In particular, different protecting groups can be used to protect different parts of one or more of the molecules involved in the reaction, particularly different OH groups of the molecule(s), and the protecting groups can be selectively removed as desired.

By way of example, suitable protecting groups for OH group(s) of sugar moieties of the diketone of general formula II may be selected from acetyl (Ac), pivaloyl (Piv) and benzoyl (Bz) groups. Suitable protecting groups for other OH group(s) of the diketone of general formula II may be selected from silyl ether protecting groups, for example t.butyldimethylsilyl (TBS), t.butyldiphenylsilyl (TBDPS), trimethylsilyl (TMS), triethylsilyl (TES) and triisopropylsilyl (TIPS). Ester and ether residues in the diketone of general formula II may not need to be protected, but if particular active substituents of the residues are present, suitable protecting groups can be selected by the person skilled in this art.

Removal of the protecting groups can be achieved in conventional manner according to the nature of the protecting group. For example, treatment of a compound including an acetylated or benzoylated sugar moiety with NaOMe in methanol at around pH 10 with stirring, for a period of time, generally 3-6 hours, at room temperature results in the complete de-O-acetylation or de-O-benzoylation. The reaction solution can then be neutralized with Amberlite IR 120 (H⁺) resin. The organic phase can then be concentrated to dryness and the residue purified on column chromatography to afford the desired deacylated material. Those skilled in the art would recognize that other standard procedures are available for the same material, such as treatment with ammonia in methanol.

### Coupling Reactions Performed on a Sugar or on a Diketone of General Formula II

Sugar-to-sugar coupling reactions may be used to prepare di-, tri- or higher saccharide sugars for coupling. A sugar molecule may be coupled to any OH group of a diketone of general formula II. Such an OH group may be a sugar OH group or a non-sugar OH group.

For these reactions, some OH groups of the reacting molecules may be protected so that the coupling is appropriately directed. Ester and ether protecting reactions performed on an OH residue of a sugar are standard organic synthesis reactions which will be familiar to those skilled in the art and require no further discussion here.

Sugar coupling reactions generally fall into two categories, according to how the sugar moiety to be coupled is activated. One general category of reaction uses a thioglycoside (e.g. thioethyl sugar or thio-2-propyl sugar) as the activated sugar moiety for coupling, in the presence of a suitable catalyst such as a catalytic amount of N-iodosuccimide (NIS) and an iodine, silyl or silver cocatalyst. The second general category of reaction uses a sugar trihaloacetimidate (e.g. trichloroacetimidate) as the activated sugar moiety for coupling. The activated sugar is coupled, via the activated carbon centre, to a glycoside acceptor, namely another sugar molecule (via an unprotected OH group thereof) or a diketone of general formula II (via an unprotected OH group thereof, including an unprotected OH group of a sugar moiety of the diketone).

The first category coupling reaction typically proceeds as follows: To a solution of thioglycoside and the glycoside acceptor in an anhydrous solvent (such as toluene, dichloromethane or ether), is added a catalytic amount of N-iodosuccimide (NIS) and trimethylsilyl trifluorosulfonate (TMSOTf). The mixture, is stirred at −20° C. to room temperature for a period of time, generally 0, 5-2 hours, and then adjusted to pH 7.0 with triethylamine. Concentration and column purification then affords the desired compound. Those skilled in the art will readily be able to identify several alternative options for this reaction of the same material, such as using NIS-I₂ or NIS-AgOTf or NIS-TfOH as catalyst.

The second category coupling reaction typically proceeds as follows: To a solution of sugar trichloroacetimidate and the glycoside acceptor in an anhydrous solvent (such as toluene, dichloromethane or an ether) is added a catalytic amount of a suitable catalyst such as trimethylsilyl trifluorosulfonate (TMSOTf), BF₃.Et₂O, or HClO₄—SiO₂. The mixture is stirred at −20° C. to room temperature for a period of time, generally 0.5-2 hours, and then adjusted to pH7.0 with triethylamine. Concentration and column purification afforded desired compound.

In the present invention, such coupling reactions may be used to couple a sugar molecule to another sugar molecule, which may be the same or a different sugar, to assemble larger sugar molecules, or to a compound of general formula I or II (including IIa and IIb) in which one or both of OR₁ and OR₃ represents OH.

For example, such coupling reactions may be used to couple a sugar molecule to another sugar molecule, which may be the same or a different sugar, to assemble larger sugar molecules, or to a compound of general formula II (including IIa and IIb) in which one or both of R₁ and R₃ represents hydrogen.

The stereochemistry of the coupling reaction is controllable, according to the portions of the sugar molecule in the vicinity of the activated C1 carbon atom of the sugar molecule. If the activated C1 carbon atom is adjacent to a C2 carbon atom which carries an O-acyl group (—O—C(O)— linkage), then the resulting bond between the C1 carbon atom of the sugar molecule and the glycoside acceptor is controlled usually to be equatorial (β) to the plane of the sugar ring. This result is found, irrespective of which category of coupling reaction is used, irrespective of whether the activating group at C1 is α or β to the plane of the sugar ring, and irrespective of whether the glycoside acceptor is another sugar molecule or a compound of general formula II in which one or both of R₁ and R₃ represents hydrogen. It is believed that this effect is caused by the coupling reaction progressing through a cyclic intermediate involving the axial (α)-O—C(O)— linkage carried by the C2 carbon atom of the sugar and the activating group at the C1 carbon atom, the cyclic intermediate being configured in such a way that coupling of the sugar to the glycoside acceptor is always such that the coupling bond is equatorial (β) to the plane of the sugar ring.

It is possible in principle for a di- or higher polysaccharide to be assembled separately from the steroid molecule, and then coupled to a steroid molecule of general formula I in which OR₁ is OH. We have found, however, that there is often a substantial reduction in the yield of the preferred 3β-saponin when a pre-assembled di- or higher polysaccharide is coupled directly to a 3ρ-OH steroidal aglycone of this type. It appears that a mixture of 3α- and 3β-saponins is typically produced, which can be impossible to separate. Therefore, it is preferred to assemble complex sugars for the desired end product of general formula I in situ on the compound of formula II, prior to converting the compound of formula II to the compound of formula I.

Coupling of a sugar moiety to a sugar moiety, whether in situ on the steroid molecule or separately from it, and coupling of a sugar moiety to a non-sugar OH group of the steroid molecule, for example the compound of formula II, can be achieved using either category of coupling.

### Ester Residues in the Compounds of General Formulae I and II

Ester residues in the compounds of general formulae I and II, including such residues introduced in place of H for R₂ as described above, may be any ester formable by reaction of an OH group with an ester-forming acid or activated derivative thereof. The organic acid may, for example, be an aliphatic carboxylic acid or amino acid. Without limitation, the organic ester group may, for example, be selected from: cathylate (ethoxycarbonyloxy), acetate, succinate, propionate, n-butyrate, i-butyrate, valerate, isovalerate, n-caproate, iso-caproate, diethylacetate, octanoate, decanoate, laurate, myristate, palmitate, stearate, benzoate, phenylacetate, phenylpropionate, cinnamate, phthalyl, glycinate, alaninate, valinate, phenylalaninate, isoleucinate, methioninate, argininate, aspartate, cysteinate, glutaminate, histidinate, lysinate, prolinate, serinate, threoninate, tryptophanate, tyrosinate, fumerate, maleate, substituted aliphatic, e.g. chloroacetate, methoxyacetate, protected amino acid ester groups, e.g. Boc-aminoglycinate (Boc=t-butoxycarbonyl), Boc-aminovalinate, CBZ-aminoglycinate (CBZ=benzyloxycarbonyl), CBZ-aminoalinate, and substituted aromatic ester groups, e.g. p-bromobenzoyloxy, m-bromobenzoyloxy, p-methoxybenzoyloxy, chlorobenzoate such as p-chlorobenzoyloxy, dichlorobenzoate such as 2,4-dichlorobenzoyloxy, nitrobenzoate such as p-nitrobenzoyloxy or 3,5-dinitrobenzoyloxy, etc.

### Ether Residues in the Compounds of General Formulae I and II

Ether residues in the compounds of general formulae I and II, may be any ether formable by reaction of an OH group of the compound of general formulae I or II, or an OH-activated form thereof, with an ether-forming compound such as an aliphatic, olefinic or cycloaliphatic hydrocarbon bearing a suitable leaving group to couple via a substitution reaction with the OH group or activated derivative thereof. The hydrocarbon may, for example, be a straight or branched alkane, alkene, alkyne, cycloalkane or cycloalkene, suitably containing up to about 15 carbon atoms, for example between 1 and about 10 carbon atoms, e.g. 1 to 6 carbon atoms. The suitable leaving group may, for example, be a halo atom such as chloro or bromo, or an organic sulfonyl leaving groups such as tosyl.

### Sugar Residues in the Compounds of General Formulae I and II

Examples of sugar residues for R₁ and R₃ include mono-, di- and tri-saccharides and higher polysaccharides and acylated forms thereof. Without limitation, such a sugar may, for example, be a mono aldose or ketose having 5 or 6 carbon atoms, preferably in the cyclised furanose or pyranose form, either as α or β anomer and having D or L optical isomerism. Examples of suitable sugars include glucose, mannose, fructose, galactose, maltose, cellobiose, sucrose, rhamnose, xyulose, arabinose, fucose, quinovose, apiose, lactose, galactose-glucose, glucose-arabinose, fucose-glucose, rhamnose-glucose, rhamnose-galactose, glucose-glucose-glucose, glucose-glucose-galactose, gluctose-rhamnose, mannose-glucose, rhamnose-(glucose)-glucose, rhamnose-(rhamnose)-glucose, glucose-(rhamnose)-glucose, glucose-(rhamnose)-galactose, glucose-(rhamnose)-rhamnose, galactose-(rhamnose)-galactose, and acylated (e.g. acetylated) derivatives thereof.

### Compositions

The compounds of general formula I, for example timosaponin BII, prepared using the method of the present invention, may be included in pharmaceutical compositions as described in the prior art and other conventional pharmaceutical forms.

### Preparation of the Compounds of General Formula II

The compounds of general formula II in which R₁ and R₃ are not sugar residues, and their protected forms, used as starting materials in the above method, can be prepared from the corresponding F-ring-closed spirostane steroidal sapogenin by an oxidative F-ring opening reaction. Such a reaction may, for example, be carried out by mixing a 3-OH-protected form of the sapogenin and NaHCO₃ in an aqueous organic solvent such as CH₂Cl₂-acetone-1.0 mM aqueous Na₂EDTA and adding (e.g. dropwise) an aqueous solution of oxone (2KHSO₅.KHSO₄.K₂SO₄).

The reaction initially provides an equilibrium mixture of the unreacted 3-O-protected sapogenin and a correspondingly 3-O-protected compound of general formula II in which OR₃═OH. Coupling of a sugar residue in place of R₃ then drives the equilibrium towards the ring-opened form (general formula II).

The sapogenin starting material is selected from sarsasapogenin, episarsasapogenin, smilagenin and epismilagenin, according to the desired stereochemistry permutations at the two chiral centres denoted by wavy lines in general formula I. To provide the permutation of stereochemistry required for timpsaponin BII (see the formula on page 1), sarsasapogenin is used.

### EXAMPLES

The following Examples are provided for further illustration of the present invention, and without in any way limiting the protection as defined by the appended claims and the foregoing description.

In the Examples, percentages and proportions of solid materials are by weight unless otherwise stated or unless the context otherwise requires. Percentages and proportions of liquid materials are by volume unless unless otherwise stated or unless the context otherwise requires. Percentages and proportions of solid materials in liquids are by weight solid to volume of liquid unless unless otherwise stated or unless the context otherwise requires. Abbreviations: NIS=N-iodosuccimide; TMSOTf=Me₃SiOTf=trimethylsilyl trifluorosulfonate; anhyd.=anhydrous; DMF=dimethylformamide; TEA triethylamine; Me=methyl; Et=ethyl; Bu=butyl; Pr=propyl. Optical rotations were determined at 25° C. with a Perkin-Elmer Model 241-Mc automatic polarimeter. ¹H NMR and ¹³C NMR were recorded with a Bruker ARX 400 spectrometer for solutions in CDCl₃ or D₂O or CD₅N. Chemical shifts are given in ppm downfield from internal Me₄Si. Mass spectra were measured using a MALDI TOF-MS with α-cyano-4-hydroxycinnamic acid (CCA) as matrix. Thin-layer chromatography (TLC) was performed on silica gel HF₂₅₄ with detection by charring with 30% (v/v) H₂SO₄ in MeOH or in some cases by UV detector. Column chromatography was conducted by elution of a column of silica gel (100-200 mesh) with EtOAc-petroleum ether (60-90° C.) as the eluent. Solutions were concentrated at <60° C. under reduced pressure.

### Example 1

### The Preparation of (25S)-26-O-β-D-glucopyranosyl-22-hydroxy-5β-furostane-3β,26-diol-3-O-β-D-glucopyranosyl-(1→2)-β-D-galactopyranoside (14)

The overall reaction scheme is as follows:

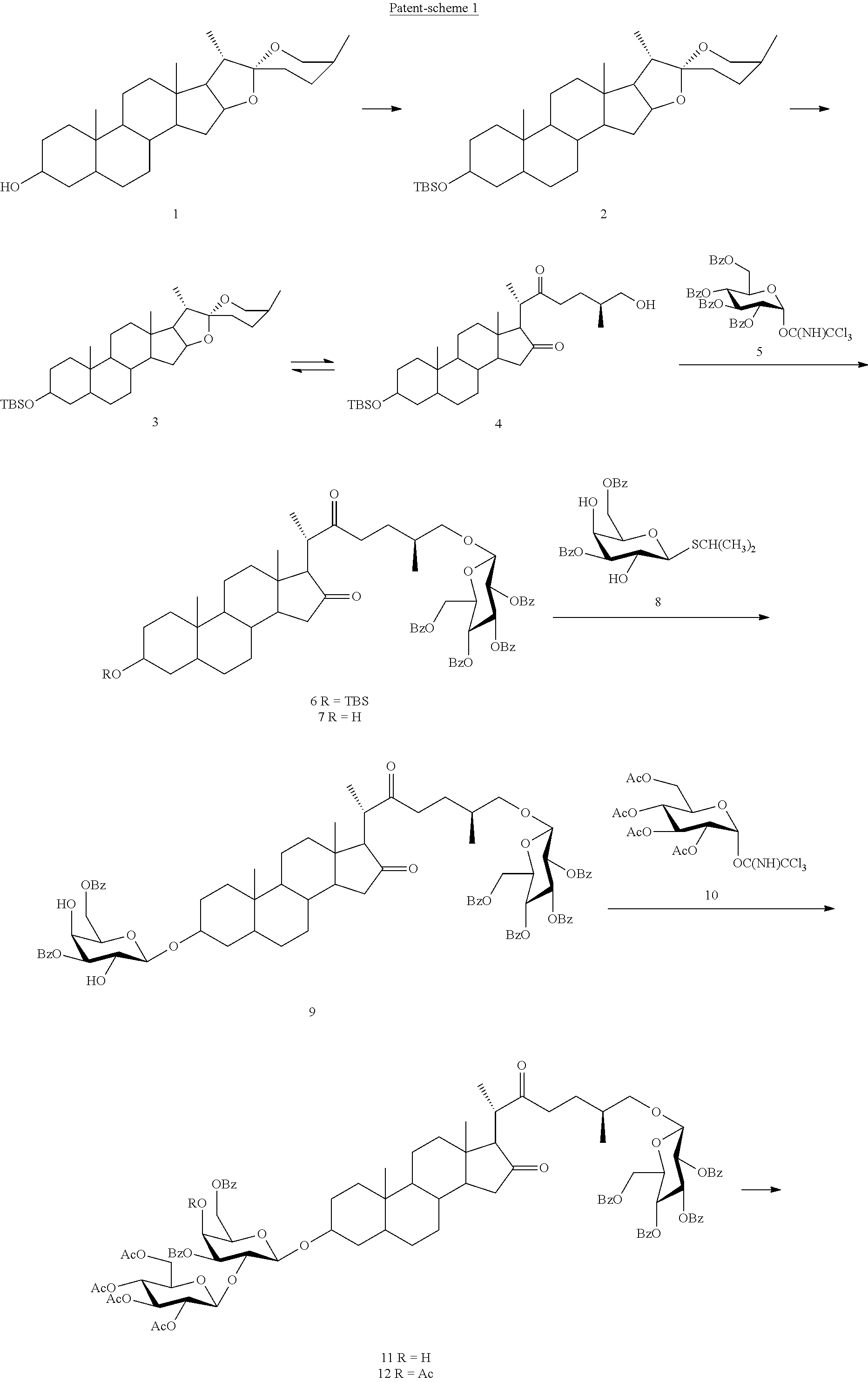

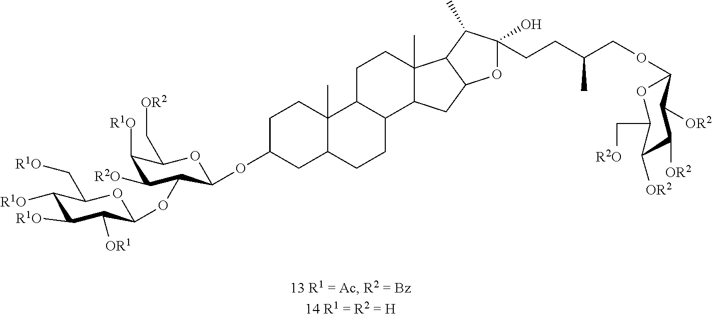

### Preparation of (2)

Compound 1 (sarsasapogenin; 13.0 g, 31.2 mmol) was dissolved in DMF (100 mL), and TBSCl (6.0 g, 39.8 mmol) was added to the solution. The mixture was stirred at 70° C.-90° C. for 8 h, at the end of which time TLC (petroleum ether-EtOAc, 30:1) indicated that all starting materials were consumed. The reaction mixture was diluted with petroleum ether (300 mL). This organic phase was washed with water (450 mL×2) and dried over anhydrous Na₂SO₄, the solid was then filtered off, and the filtrate was concentrated under vacuum to give a white solid 2 (16.1 g, 97%): ¹H NMR (400 MHz, CDCl₃): δ4.37-4.42 (m, 1H, H-16), 4.01 (br s, 1H, H-3), 3.95 (dd, 1H, J 2.6, 10.9 Hz, H-26a), 3.29 (d, 1H, J=11.8 Hz, H-26b), 1.07 (d, 3H, J 7.1 Hz, 21-CH₃), 0.98 (d, 3H, J 6.7 Hz, 27-CH₃), 0.94 (s, 3H, 19-CH₃), 0.87 (s, 9H, t-Bu), 0.75 (s, 3H, 18-CH₃), 0.001 (s, 6H, 2 CH₃). ¹³C NMR (100 MHz, CDCl₃): δ109.6, 81.0, 67.3, 65.0, 62.1, 56.5, 42.1, 40.6, 40.0, 36.4, 35.3, 35.1, 27.0, 26.8, 26.7, 25.8, 25.7, 23.9, 20.9, 18.0, 16.4, 16.0, 14.3.

### Preparation of (3) and (4)

To a mixture of compound 2 (16.1 g, 30 mmol) and NaHCO₃ (49 g, 0.58 mmol) in CH₂Cl₂-acetone-1.0 mM aqueous Na₂EDTA (450 mL, 1:1:1, v/v/v) was added dropwise a solution of Oxone (2 KHSO₅.KHSO₄.K₂SO₄) (105 g, 0.169 mol) in 25 mL of 1.0 mM aqueous Na₂EDTA. The mixture was stirred at room temperature overnight. TLC (petroleum ether-EtOAc, 10:1) indicated that all starting materials were consumed. The mixture was concentrated, then diluted with CH₇Cl₂. The organic phase was washed with water, dried over anhydrous Na₂SO₄, concentrated, and purified by column chromatography (petroleum ether-EtOAc, 20:1), to afford 3 and 4 as a mixture of white solid (13.5 g, 81%).

### Preparation of (6)

To a mixture of compounds 3 and 4 (2.25 g, 4.1 mmol) and 5 (3.56 g, 4.8 mmol, commercially available) in anhyd CH₂Cl₂ (36 mL) was added Me₃SiOTf (86 μL, 0.47 mmol) under N₂ atmosphere at −20° C. The mixture was stirred under these conditions for 40 min, at the end of which time TLC (petroleum ether-EtOAc, 4:1) indicated that all starting materials were consumed. The reaction mixture was neutralized with triethylamine (TEA), then concentrated. Column chromatography (petroleum ether-EtOAc, 6:1) of the residue gave 6 as a foamy solid (3.7 g, 80%): ¹H NMR (400 MHz, CDCl₃): δ8.02-7.27 (m, 20H, 4 PhCO), 5.90 (t, 1H, J 9.6 Hz, H-3^{Glc}), 5.67 (t, 1H, J 9.7 Hz, H-4^{Glc}), 5.53 (dd, 1H, J 7.8, 9.8 Hz, H-2^{Glc}), 4.86 (d, 1H, J 7.8 Hz, H-1^{Glc}), 4.63 (dd, 1H, J 3.3, 12.1 Hz, H-6a^{Glc}), 4.51 (dd, 1H, J 5.3, 12.1 Hz, H-6b^{Glc}), 4.19-4.14 (m, 1H, H-5^{Glc}), 4.05 (br.s, 1H, H-3^{Sar}), 3.87 (dd, 1H, J 5.3, 9.4 Hz, H-26a^{Sar}), 3.31 (dd, 1H, J 7.3, 9.4 Hz, H-26b^{Sar}), 0.95 (s, 3H, 19-CH₃), 0.89 (d, 3H, J 6.6 Hz, 21-CH₃), 0.88 (s, 9H, t-Bit of TBS), 0.79 (d, 3H, J 6.6 Hz, 27-CH₃), 0.72 (s, 3H, 18-CH₃), 0.01 (s, 6H, 2 CH₃). ¹³C NMR (400 MHz, CDCl₃): δ218.4, 213.6, 166.1, 165.8, 165.2, 165.1, 133.3, 133.1, 133.1, 133.0, 129.7, 129.7, 129.5, 129.3, 128.8, 128.7, 128.3, 128.3, 128.5, 101.4, 75.3, 72.9, 72.0, 71.9, 69.8, 67.2, 66.4, 63.2, 51.2, 43.2, 42.0, 39.8, 39.5, 39.1, 37.2, 36.3, 35.1, 34.7, 34.3, 32.6, 29.6, 28.5, 26.7, 26.6, 26.5, 25.9, 25.8, 25.8, 25.6, 23.8, 22.6, 20.5, 18.0, 16.8, 15.3, 13.1, −4.86, −4.89.

### Preparation of (7)

To a solution of compound 6 (3.6 g, 3.2 mmol) in dry CH₂Cl₂ (40 mL) was added BF₃.Et₂O (1.0 mL, 7.3 mmol), and the mixture was stirred at room temperature for 4 h, and TLC (petroleum ether-EtOAc, 1:1) indicated that the reaction was complete. The mixture was diluted with CH₂Cl₂, washed with satd aq NaHCO₃ and then satd aq NaCl. The organic layer was combined, dried, and concentrated. Purification by column chromatography (petroleum ether-EtOAc, 1:1) gave 7 as a white foamy solid (3.07 g, 95%): ¹H NMR (400 MHz, CDCl₃): δ8.02-7.27 (m, 20H, 4 PhCO), 5.89 (t, 1H, J 9.6 Hz, H-3^{Glc}), 5.67 (t, 1H, J 9.7 Hz, H-4^{Glc}), 5.53 (dd, 1H, J 7.8, 9.8 Hz, H-2^{Glc}), 4.84 (d, 1H, J 7.8 Hz, H-1^{Glc}), 4.62 (dd, 1H, J 3.3, 12.0 Hz, H-6a^{Glc}), 4.50 (dd, 1H, J 5.1, 12.0 Hz, H-6b^{Glc}), 4.17-4.12 (m, 2H, H-5^{Glc}, H-3^{Sar}), 3.88 (dd, 1H, J 5.2, 9.4 Hz, H-26a^{Sar}), 3.29 (dd, 1H, J 7.4, 9.3 Hzm H-26b^{Sar}), 0.98 (s, 3H, 19-CH₃), 0.94 (d, 3H, J 6.6 Hz, 21-CH₃), 0.81 (d, 3H, J 6.6 Hz, 27-CH₃), 0.73 (s, 3H, 18-CH₃). ¹³C NMR (100 MHz, CDCl₃): 218.1, 213.4, 166.0, 165.7, 165.1, 165.0, 133.3, 133.1, 133.0, 133.0, 129.7, 129.6, 129.6, 129.6, 129.5, 129.3, 128.8, 128.8, 128.3, 128.2, 128.2, 101.5, 75.28, 72.9, 72.0, 71.9, 69.9, 66.7, 66.4, 63.2, 60.3, 51.1, 43.2, 42.0, 39.6, 39.5, 39.0, 38.9, 37.1, 36.2, 35.1, 34.6, 33.4, 32.6, 29.5, 29.5, 29.4, 27.7, 26.7, 26.3, 26.3, 23.7, 22.6, 20.5, 19.1, 16.8, 15.2, 14.1, 13.1.

### Preparation of (9)

To a mixture of compound 8 (1.38 g, 3.08 mmol, J. Org. Chem. 2004, 69, 5497-5500) and 7 (2.6 g, 2.6 mmol) in anhyd CH₂Cl₂ (50 mL) was added NIS (1.04 g, 4.6 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 9 as a foamy solid (2.94 g, 82%): ¹H NMR (400 MHz, CDCl₃): δ8.11-7.28 (m, 30H, 6 PhCO), 5.90 (t, 1H, J 9.6 Hz, II-3^{Glc}), 5.67 (t, 1H, J 9.0 Hz, H-4^{Glc}), 5.53 (dd, 1H, J 7.8, 9.8 Hz, H-2^{Glc}), 5.18 (dd, 1H, J 3.2, 10.0 Hz, H-3^{Gal}), 4.84 (d, 1H, J 7.7 Hz, H-1^{Glc}), 4.64-4.48 (m, 4H, H-6a^{Glc}, H-6a^{Gal}, H-6b^{Glc}, H-6b^{Gal}), 4.48 (d, 1H, J 7.8 Hz, H-1^{Gal}), 4.23 (br.d, 1H, J 3.0 Hz, H-4^{Gal}), 4.17-4.15 (m, 1H, H-5^{Gal}), 4.10 (br.s, 1H, H-3^{Sar}), 4.05 (dd, 1H, J 7.7, 10.0 Hz, H-5^{Glc}), 3.96 (t, 1H, J 6.6 Hz, H-2^{Gal}), 3.88 (dd, 1H, J 5.2, 9.4 Hz, H-26a^{Snr}), 3.30 (dd, 1H, J 7.4, 9.3 Hz, H-26b^{Sar}), 0.95 (s, 3H, 19-CH₃), 0.94 (d, 3H, J 7.3 Hz, 21-CH₃), 0.82 (d, 3H, J 6.6 Hz, 27-CH₃), 0.73 (s, 3H, 18-CH₃).

### Preparation of (12)

To a mixture of compound 10 (685 mg, 1.39 mmol) and 9 (1.6 g, 1.15 mmol) in anhyd CH₂Cl₂ (15 mL) was added Me₃SiOTf (26 μL, 0.14 mmol) under N₂ atmosphere at −42° C. The mixture was stirred under these conditions for 30 min, at which time TLC (petroleum ether-EtOAc, 1:1) indicated that all starting materials were consumed. The reaction mixture was neutralized with TEA, then concentrated to afford a syrup. The syrup was dissolved in pyridine (15 mL) and Ac₂O (5 mL) and stirred at room temperature for about 3 h. Then he mixture was concentrated with toluene and purified by column chromatography (petroleum ether-EtOAc, 3:1) to give 12 as a white foamy solid (1.5 g, 75%): ¹H NMR 400 MHz, CDCl₃): δ8.01-7.28 (m, 30H, 6 PhCO), 5.90 (t, 1H, J 9.6 Hz, H-3^{Glc1}), 5.67 (t, 1H, J 9.6 Hz, H-4^{Glc1}), 5.58 (dd, 1H, J 3.4, 4.1 Hz, H-4^{Gal}), 5.53 (dd, 1H, J 7.8, 9.7 Hz, H-2^{Glc1}), 5.30 (dd, 1H, J 3.5, 9.9 Hz, H-3^{Gal}), 4.99 (t, 1H, J 9.2 Hz, H-3^{Glc□}), 4.92 (t, 1H, J 9.3 Hz, H-4^{Glc□}), 4.85 (dd, 1H, J 7.8, 9.8 Hz, H-2^{Glc□}), 4.84 (d, 1H, J 9.8 Hz, H-1^{Glc□}), 4.70 (d, 1H, J 7.6 Hz, H-1^{Glcf}), 4.61 (dd, 1H, J 8.8, 12.0 Hz, H-6a), 4.56 (d, H-1, J 7.7 Hz, H-1-1^{Gal}), 4.53-4.48 (m, 2H, H-6a′, H-6b), 4.36 (dd, 1H, J 7.2, 12.1 Hz, H-6a″), 4.29 (dd, 1H, J 6.8, 11.1 Hz, H-6b′), 4.19-4.12 (m, 1H, H-5), 4.10-4.04 (m, 4H, H-6b″, H-3^{Sar}, H-5′, H-2^{Gal}), 3.90 (dd, 1H, J 5.2, 9.4 Hz, H-26a^{Sar}), 3.72-3.69 (m, 1H, H-5″), 3.30 (dd, 1H, J 7.4, 9.3 Hz, H-26b^{Sar}), 2.14, 2.08, 1.98, 1.89, 1.75 (5 s, 5×3 CH₃CO), 0.99 (s, 3H, 19-CH₃), 0.94 (d, 3H, J 7.3 Hz, 21-CH₃), 0.82 (d, 3H, J 6.6 Hz, 27-CH₃), 0.73 (s, 3H, 18-CH₃). ¹³C NMR 100 MHz, CDCl₃): δ218.0, 213.3, 170.5, 169.9, 169.7, 169.3, 169.2, 166.0, 165.9, 165.7, 165.3, 165.1, 165.0, 133.6, 133.3, 133.2, 133.1, 133.0, 133.0, 129.7, 129.6, 129.6, 129.5, 129.5, 129.5, 129.5, 129.4, 129.4, 129.3, 129.3, 129.1, 128.8, 128.6, 128.3, 128.3, 128.3, 128.2, 128.2, 101.4, 100.9, 99.5, 75.4, 75.2, 75.1, 73.9, 72.9, 72.8, 72.0, 71.9, 71.6, 71.1, 70.6, 69.9, 69.5, 68.7, 67.5, 66.4, 63.2, 62.5, 61.8, 60.2, 51.1, 43.2, 42.0, 41.3, 39.8, 39.5, 39.0, 37.1, 35.9, 34.9, 34.9, 34.6, 32.6, 29.7, 29.6, 26.7, 26.3, 26.2, 26.1, 25.6, 23.7, 22.5, 20.9, 20.7, 20.6, 20.5, 20.4, 20.1, 16.8, 15.2, 14.1, 13.0.

### Preparation of (13)

The mixture of compound 12 (150 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 13 as a white solid (102 mg, 68%); NMR (400 MHz, CDCl₃): δ8.00-7.28 (m, 30H, 6 PhCO), 5.88 (t, 1H, J 9.6 Hz, H-3^{Glcl}), 5.67 (t, 1H, J 9.7 Hz, H-4^{Glcl}), 5.58 (dd, 1H, J 3.4, 4.1 Hz, H-4^{Gal}), 5.53 (dd, 1H, J 7.8, 9.7 Hz, H-2^{Glcl}), 5.30 (dd, 1H, J 3.7, 9.8 Hz, H-3^{Gal}″), 5.00 (t, 1H, J 9.6 Hz, H-3^{Glc□}), 4.93 (t, 1H, J 9.3 Hz, H-4^{Glc□}), 4.86 (dd, 1H, J 7.7, H-2^{Glc□}), 4.81 (d, 1H, J 7.8 Hz, H-1^{Glc□}), 4.70 (d, 1H, J 7.6 Hz, H-1^{Glcl}), 4.61 (dd, 1H, J 3.0, 12.1 Hz, H-6a), 4.55 (d, 1H, J 7.5 Hz, H-1^{Gal}), 4.53-4.51 (m, 2H, H-6a′, H-6b), 4.40 (dd, 1H, J 7.2, 12.1 Hz, H-6a″), 4.30 (dd, 1H, J 6.8, 11.1 Hz, H-6b′), 4.14-4.03 (m, 5H, H-5, H-6b″, H-3^{Sar}, H-5′, H-2^{Gal}), 3.87 (dd, 1H, J 5.2, 9.4 Hz, H-26a^{Sar}), 3.75-3.48 (m, 1H, 1H-5″), 3.25 (dd, 1H, J 7.4, 9.3 Hz, H-26b^{Sar}, 2.14, 2.09, 1.98, 1.90, 1.75 (5 s, 5×3 CH₃CO), 0.99 (s, 3H, 19-CH₃), 0.94 (d, 3H, J 7.3 Hz, 21-CH₃), 0.80 (d, 3H, J 6.6 Hz, 27-CH₃), 0.73 (s, 3H, 18-CH₃).

### Preparation of (14)

Compound 13 (100 mg, 0.057 mmol) was dissolved in anhyd CH₂Cl₂-MeOH (1:2, v/v, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 14 as an amorphous solid (50 mg, 95%): NMR (400 MHz, C₆D₅N): δ5.27 (d, 1H, J 7.5 Hz, H-1^{Glc□}), 4.91 (d, 1H, J 7.5 Hz, H-1^{Gal}), 4.82 (d, 1H, J 7.6 Hz, H-1^{Glcl}), 1.15 (d, 3H, J 6.9 Hz, 21-CH₃), 1.10 (s, 3H, 19-CH₃), 1.02 (d, 3H, J 6.5 Hz, 27-CH₃), 0.98 (s, 3H, 18-CH₃). ¹³C NMR 400 MHz, CD₅N): δ110.5, 105.8, 104.9, 102.3, 81.5, 81.0, 78.4, 78.2, 78.2, 77.8, 76.7, 76.4, 75.3, 75.2, 75.1, 75.0, 71.5, 71.5, 69.6, 63.8, 62.6, 62.0, 56.2, 41.0, 40.5, 40.2, 40.1, 36.9, 36.7, 35.3, 35.1, 34.2, 32.2, 30.7, 30.7, 28.1, 26.8, 26.6, 26.6, 23.8, 21.0, 17.3, 16.5, 16.3. MALDITOF-MS: Calcd for C₄₅H₇₆O₁₉: 920.5 [M]⁺. Found 944.0 [M+Na]⁺.

### Example 2

### The Preparation of (25S)-26-O-β-D-glucopyranosyl-22-hydroxy-5β-furostane-3β,26-diol-3-O-β-D-galactopyranoside (18)

The overall reaction scheme is as follows:

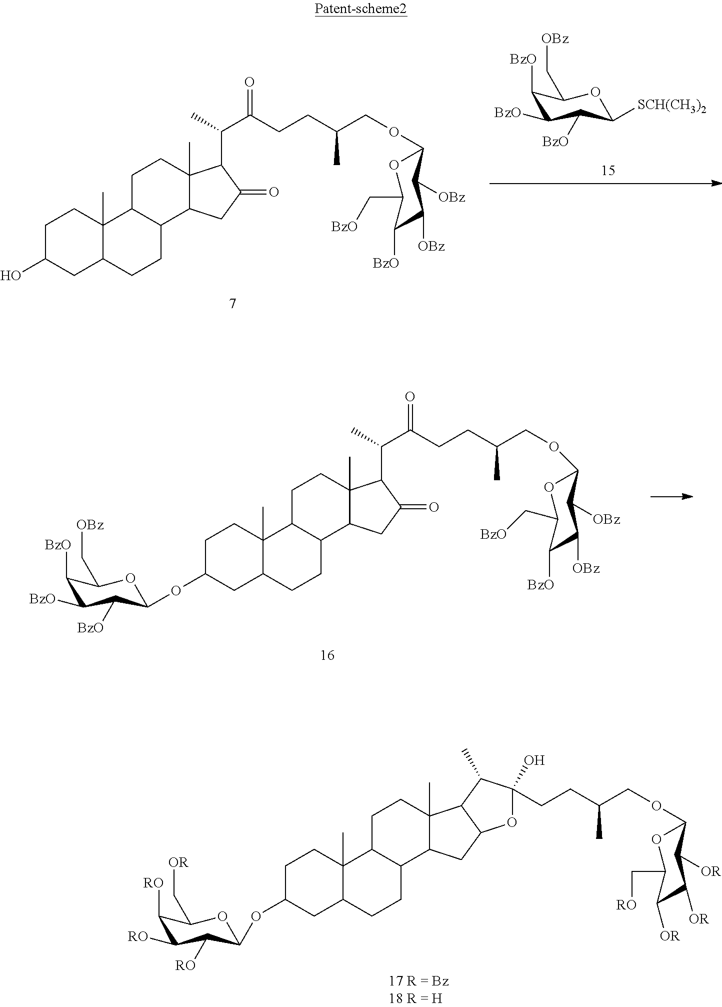

### Preparation of (16)

To a mixture of compound 15 (2.03 g, 3.10 mmol, commercially available) and 7 (2.6 g, 2.6 mmol) in anhyd CH₂Cl₂ (50 mL) was added MS (1.05 g, 4.7 mmol), Me₃SiOTf (56 μL, 0.31 mmol) under N₂ atmosphere at 0° C. The mixture was stirred under r.t. for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 16 as a foamy solid (3.72 g, 90%): MALDITOF-MS: Calcd for C₉₅H₉₆O₂₂: 1588.64 [M]⁺. Found 1611.5 [M+Na]⁺.

### Preparation of (17)

The mixture of compound 16 (600 mg, 038 mmol) and NaBH₄ (357 mg, 9.4 mmol) in 2-propanol (16 mL) and CH₂Cl₂ (2 mL) was stirred at room temperature for about 7.5 h. The reaction mixture was then extracted with CH₂Cl₂ (100 mL 2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 17 as a white solid (393 mg, 65%): Calcd for C₉₅H₉₈O₂₂: 1590.65 [M]⁺. Found 1613.5 [M+Na]⁺.

### Preparation of (18)

Compound 17 (210 mg, 0.13 mmol) was dissolved in anhyd CH₂Cl₂-MeOH (1:2, 24 mL), and then 1.0 M NaOMe in MeOH (0.25 mL) was added at 0° C. After stirring at room temperature for 5 h, TLC (n-BuOH-EtOH—H₂O, 2:0.5:0.5) indicated that the reaction was complete. The solution was neutralized with ion-exchange resin (H⁺), and then filtered and concentrated. The residue was purified by Bio-gel P₂ column to afford 18 as an amorphous solid (94 mg, 95%): Selected ¹H NMR (400 MHz, C₆D₅N): S 5.35 (d, 1 H, J 7.5 Hz, H-1^{Glc□}), 5.00 (d, 1H, J 8.0 Hz, H-1^{Gal}), ¹³C NMR 100 MHz, CD₅N): δ104.9 (C-1), 102.3 (C-1). MALDITOF-MS: Calcd for C₃₉H₆₆O₁₄: 758.45 [M]⁺. Found 781.31 [M+Na]⁺.

### Example 3

### The Preparation of (25S)-26-O-β-D-glucopyranosyl-22-hydroxy-5β-furostane-3β,26-diol-3-O-β-D-glucopyranoside (21)

The overall reaction scheme is as follows:

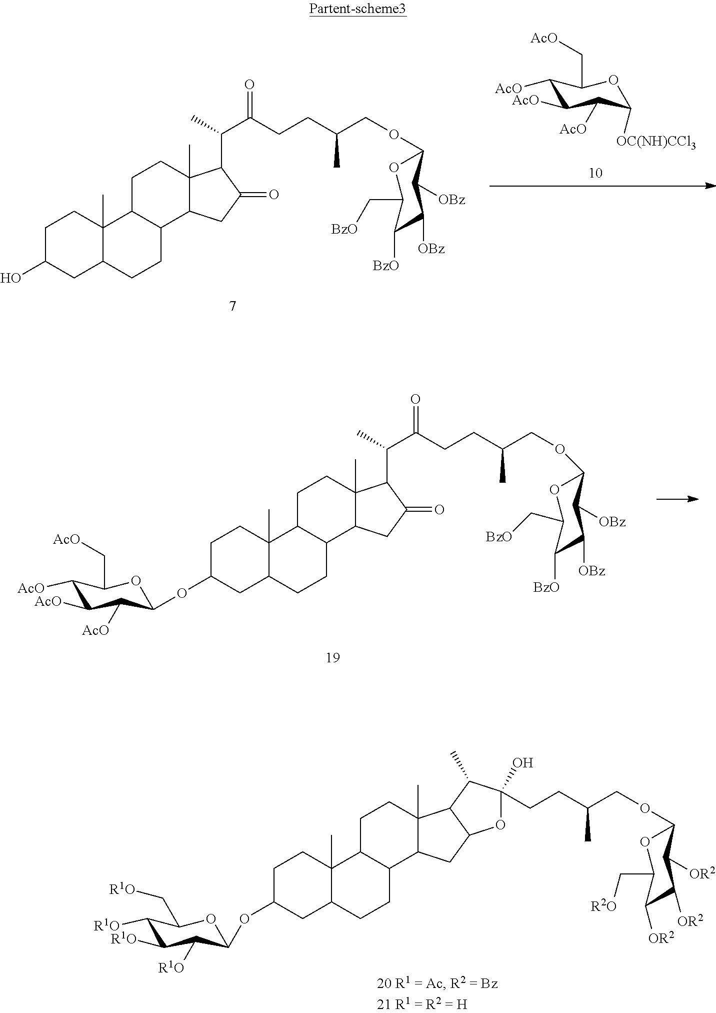

### Preparation of (19)

To a mixture of compound 10 (1.54 g, 3.12 mmol) and 7 (2.6 g, 2.6 mmol) in anhyd CH₂Cl₂ (30 mL) was added Me₃SiOTf (56 μL, 0.31 mmol) under N₂ atmosphere at 0° C. The mixture was stirred under room temperature for 15 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 19 as a foamy solid (2.96 g, 85%): MALDITOF-MS: Calcd for C₇₅H₈₈O₂₂: 1340.58 [M]⁺. Found 1363.70 [M+Na]⁺.

### Preparation of (20)

The mixture of compound 19 (114 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 20 as a white solid (74 mg, 65%): MALDITOF-MS: Calcd for C₇₅H₉₀O₂₂: 1342.59 [M]⁺. Found 1365.60 [M+Na]⁺.

### Preparation of (21)

Compound 20 (70 mg, 0.052 mmol) was dissolved in anhyd CH₂Cl₂-MeOH (1:2, v/v, 18 mL), and then 1.0 M NaOMe in MeOH (0.2 mL) was added at 0° C. After stirring at room temperature for 5.5 h, TLC (n-BuOH-EtOH—H₂O, 2:0.5:0.5) indicated that the reaction was complete. The solution was neutralized with ion-exchange resin (H⁺), and then filtered and concentrated. The residue was purified by Bio-gel P₂ column to afford 21 as an amorphous solid (38 mg, 96%): Selected ¹H NMR (400 MHz, C₆D₅N): δ5.36 (d, 1 H-1, J 7.5 Hz, H-1^{GlcII}), 5.20 (d, 1H, J 7.9 Hz, H-1^{Gal}), ¹³C NMR 100 MHz, CD₅N): δ105.1 (C-1), 103.8 (C-1). MALDITOF-MS: Calcd for C₃₉H₆₆O₁₄: 758.45 [M]⁺. Found 781.31 [M+Na]⁺.

### Example 4

### The Preparation of (25S)-26-O-β-D-glucopyranosyl-22-hydroxy-5β-furostane-3β,26-diol-3-O-β-D-glucopyranosyl-(1→4)-β-D-galactopyranoside (26)

The overall reaction scheme is as follows:

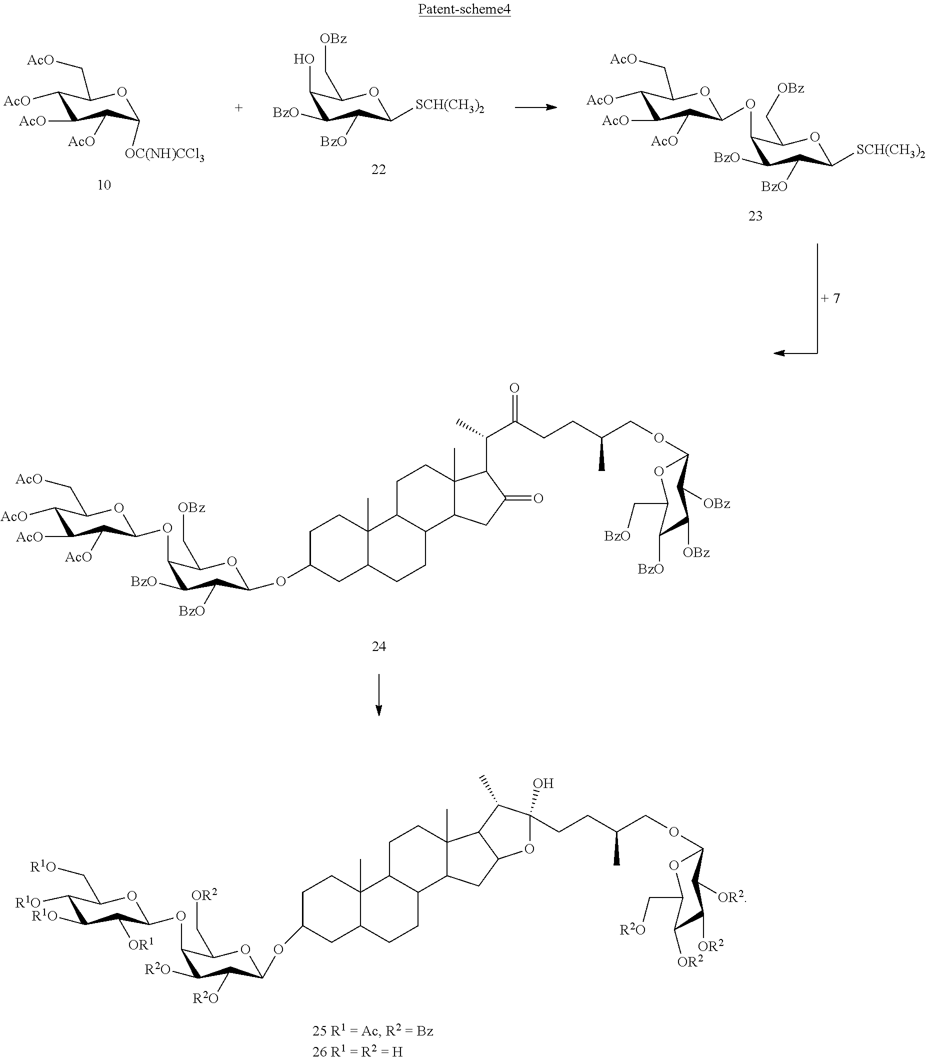

### Preparation of (24)

To a mixture of compound 10 (2.5 g, 5.1 mmol, commercially available from Beijing Carbomex Biotech Co., Ltd.) and 22 (2.55 g, 4.6 mmol) in anhyd CH₂Cl₂ (50 mL) at −20° C. was added TMSOTf (92 μL, 0.51 mmol). The mixture was stirred at these conditions for 1 h, then neutralized with TEA, and concentrated. The residue was purified by column chromatography to give a foamy solid 23 (3.36 g, 83%). To a mixture of 23 (3.1 g, 3.52 mmol) and 7 (3.24 g, 3.2 mmol) in anhyd CH₂Cl₂ (55 mL) at −20° C. was added NIS (1.18 g, 5.28 mmol) and Me₃SiOTf (63 μL, 0.35 mmol) under N₂ atmosphere at −20° C. The mixture was stirred under these conditions for 30 min, at the end of which time TLC (petroleum ether-EtOAc, 3:2) indicated that all starting materials were consumed. The reaction mixture was neutralized with TEA, then concentrated. Column chromatography (petroleum ether-EtOAc, 2:1) of the residue gave 24 as a foamy solid (4.94 g, 85%): MALDITOF-MS: Calcd for C₁₀₂H₁₁₀O₃₀: 1814.71 [M]⁺. Found 1837.50 [M Na]⁺.

### Preparation of (25)

The mixture of compound 24 (500 mg, 0.275 mmol) and NaBH₄ (313 mg, 8.26 mmol) in 2-propanol (24 mL) and CH₂Cl₂ (3 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 25 as a white solid (320 mg, 64%): MALDITOF-MS: Calcd for C₁₀₂H₁₁₂O₃₀: 1816.72 [M]⁺. Found 1839.50 [M+Na]⁺.

### Preparation of (26)

Compound 25 (200 mg, 0.11 mmol) was dissolved in anhyd CH₂Cl₂-MeOH (1:2, v/v, 21 mL), and then 1.0 M NaOMe in MeOH (0.22 mL) was added at 0° C. After stirring at room temperature for 4.5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 26 as an amorphous solid (95 mg, 94%): Selected ¹H NMR (400 MHz, C₆D₅N): δ 5.36 (d, 1H, J 7.5 Hz, H-1), 5.30 (d, 1H, J 7.5 Hz, H-1), 5.20 (d, 1H, J 7.9 Hz, H-1), ¹³C NMR 100 MHz, CD₅N): δ 105.1 (C-1), 103.8 (C-1), 102.5 (C-1). MALDITOF-MS: Calcd for C₄₅H₇₆O₁₉: 920.5 [M]⁺. Found 943.7 [M Na]⁺.

### Example 5

### The Preparation of (25S)-26-O-β-D-glucopyranosyl-22-hydroxy-5β-furostane-3β,26-diol-3-O-β-D-glucopyranosyl-(1→2)-β-D-glucopyranoside (31)

The overall reaction scheme is as follows:

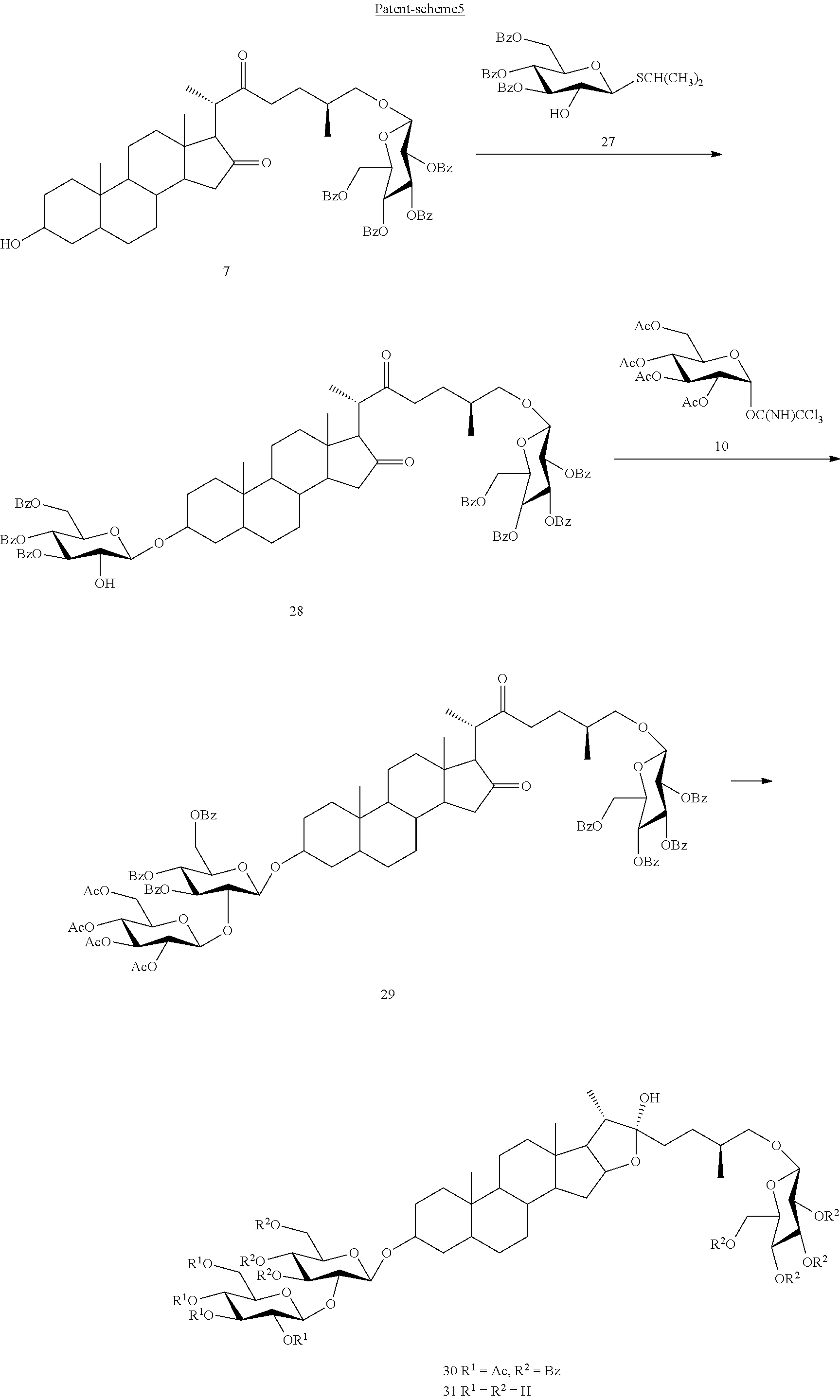

### Preparation of (28)

To a mixture of compound 27 (1.57 g, 2.86 mmol, J. Org. Chem. 2004, 69, 5497-5500) and 7 (2.6 g, 2.6 mmol) in anhyd CH₂Cl₂ (35 mL) was added NIS (643 mg, 2.86 mmol) and Me₃SiOTf (55 μl, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 28 as a foamy solid (3.09 g, 80%), MALDITOF-MS: Calcd for C₈₈H₉₂O₂₁: 1484.61 [M]⁺. Found 1507.80 [M Na]⁺.

### Preparation of (29)

To a mixture of compound 10 (834 mg, 1.69 mmol) and 28 (2.1 g, 1.41 mmol) in anhyd CH₂Cl₂ (18 mL) was added Me₃SiOTf (31 μL, 0.17 mmol) under N₂ atmosphere at −20° C. The mixture was stirred under these conditions for 20 min, at which time TLC (petroleum ether-EtOAc, 1:1) indicated that all starting materials were consumed. The reaction mixture was neutralized with TEA, then concentrated to afford 29 as a white foamy solid (2.0 g, 78%). MALDITOF-MS: Calcd for C₁₀₂H₁₁₀O₃₀: 1814.71 [M]⁺. Found 1837.8 [M+Na]⁺.

### Preparation of (30)

The mixture of compound 29 (600 mg, 0.33 mmol) and NaBH₄ (312 mg, 8.25 mmol) in 2-propanol (24 mL) and CH₂Cl₂ (3 mL) was stirred at room temperature for about 6.5 h. The reaction mixture was then extracted with CH₂Cl₁, (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 30 as a white solid (313 mg, 67%). MALDITOF-MS: Calcd for C₁₀₂H₁₁₂O₃₀: 1816.71 [M]⁺. Found 1839.8 [M+Na]⁺.

### Preparation of (31)

Compound 30 (200 mg, 0.14 mmol) was dissolved in anhyd CH₂Cl₂-MeOH (1:2, 27 mL), and then 1.0 M NaOMe in MeOH (0.3 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 31 as an amorphous solid (122 mg, 95%): Selected ¹H NMR (400 MHz, C₆D₅N): δ5.36 (d, 1H, J 7.8 Hz, H-1), 5.30 (d, 1H, J 7.5 Hz, H-1), 5.20 (d, 1H, J 7.9 Hz, H-1), ¹³C NMR 100 MHz, CD₅N): δ 104.9 (C-1), 103.5 (C-1), 102.1 (C-1). MALDITOF-MS: Calcd for C₄₅H₇₆O₁₉: 920.5 [M]⁺. Found 944.0 [M Na]⁺.

### Example 6

### The Preparation of (25S)-26-O-β-D-glucopyranosyl-22-hydroxy-5β-furostane-3β,26-diol-3-O-β-D-glucopyranosyl-(1→4)-β-D-glucopyranoside (36)

The overall reaction scheme is as follows:

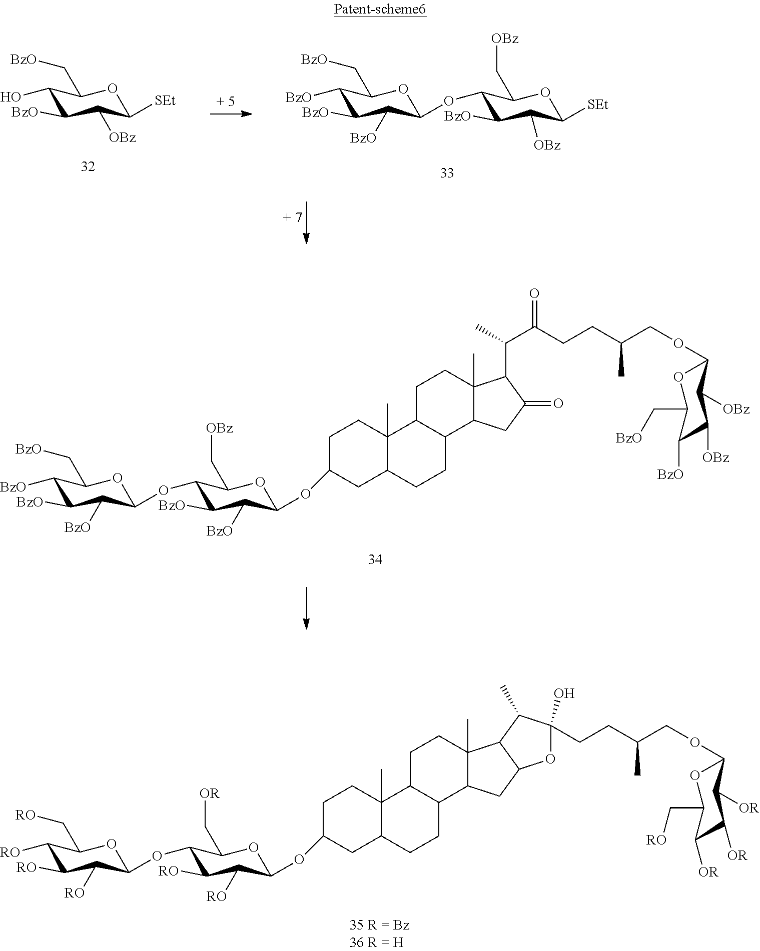

### Preparation of (34)

To a mixture of compound 32 (3.1 g, 5.78 mmol, J. Org. Chem. 2004, 69, 5497-5500) and 5 (5.14 g, 6.94 mmol) in anhyd CH₂Cl₂ (60 mL) at 0° C. was added TMSOTf (126 μL, 0.70 mmol). The mixture was stirred at these conditions for 0.5 h, then neutralized with TEA, and concentrated. The residue was purified by column chromatography to give a foamy solid 33 (5.8 g, 90%). To a mixture of 33 (2.0 g, 1.8 mmol) and 7 (1.66 g, 1.64 mmol) in anhyd CH₂Cl₂ (30 mL) was added NIS (607 mg, 2.7 mmol) and Me₃SiOTf (33 μL, 0.18 mmol) under N₂ atmosphere at 0° C. The mixture was stirred under room temperature for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 34 as a foamy solid (2.88 g, 85%). MALDITOF-MS: Calcd for C₁₂₂H₁₁₈O₃₀: 2062.77 [M]⁺. Found 2085.90 [M+Na]⁺.

### Preparation of (35)

The mixture of compound 34 (175 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 35 as a white solid (120 mg, 68%). MALDITOF-MS: Calcd for C₁₂₂H₁₂₀O₃₀: 2064.77 [M]⁺. Found 2087.90 [M+Na]⁺.

### Preparation of (36)

Compound 35 (120 mg, 0.057 mmol) was dissolved in anhyd CH₂Cl₂-MeOH (1:2, 18 mL), and then 1.0 M NaOMe in MeOH (0.21 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 36 as an amorphous solid (50 mg, 95%): Selected ¹H NMR (400 MHz, C₆D₅N): δ5.36 (d, 1H, J 7.8 Hz, H-1), 5.30 (d, 1H, J 7.5 Hz, H-1), 5.20 (d, 1H, J 7.9 Hz, H-1), ¹³C NMR 100 MHz, CD₅N): δ 104.9 (C-1), 103.5 (C-1), 102.1 (C-1). MALDITOF-MS: Calcd for C₄₅H₂₆O₁₉: 920.50 [M]⁺. Found 943.90 [M+Na]⁺,

### Example 7

### The Preparation of (25S)-26-O-β-D-glucopyranosyl-22-hydroxy-5β-furostane-3β,26-diol-3-O-β-D-glucopyranosyl-(1→6)-β-D-glucopyranoside (41)

The overall reaction scheme is as follows:

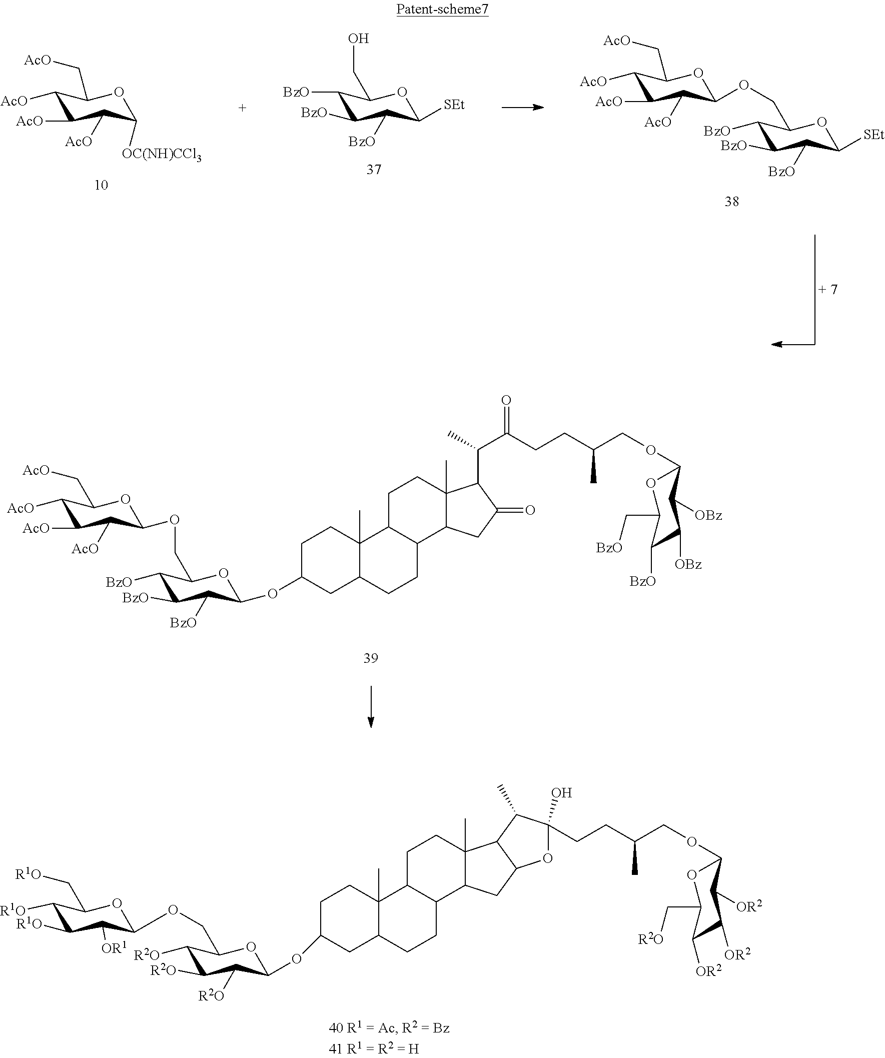

### Preparation of (39)

To a mixture of compound 10 (2.5 g, 5.07 mmol) and 37 (2.47 g, 4.61 mmol) in anhyd CH₂Cl₂ (50 mL) at 0° C. was added TMSOTf (92 μL, 0.51 mmol). The mixture was stirred at these conditions for 20 min, then neutralized with TEA, and concentrated. The residue was purified by column chromatography to give a foamy solid 38 (3.67 g, 92%). To a mixture of 38 (2.1 g, 2.4 mmol) and 7 (2.02 g, 2.0 mmol) in anhyd CH₂Cl₂ (35 mL) at −20° C. was added MS (810 mg, 3.6 mmol) and Me₃SiOTf (43 μL, 0.24 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 39 as a foamy solid (2.98 g, 82%). MALDITOF-MS: Calcd for C₁₀₂H₁₁₀O₃₀: 1814.71 [M]⁺. Found 1837.8 [M+Na]⁺.

### Preparation of (40)

The mixture of compound 39 (154 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 40 as a white solid (99 mg, 64%). MALDITOF-MS: Calcd for C₁₀₂H₁₁₂O₃₀: 1816.71 [M]⁺. Found 1839.8 [M+Na]⁺.

### Preparation of (36)

Compound 40 (80 mg, 0.056 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 41 as an amorphous solid (50 mg, 96%): ¹H NMR (400 MHz, C₆D₅N): δ5.31 (d, 1H, J 7.5 Hz, H-1), 5.28 (d, 1H, J 7.5 Hz, H-1), 5.19 (d, 1H, J 7.6 Hz, H-1). ¹³C NMR (100 MHz, CD₅N): δ105.0, 103.5, 102.1. MALDITOF-MS: Calcd for C₄₅H₇₆O₁₉: 920.5 [M]⁺. Found 943.8 [M Na]⁺.

### Example 8

### The Preparation of (25S)-26-O-β-D-glucopyranosyl-22-hydroxy-5β-furostane-3β,26-diol-3-O-α-L-rhamnopyranosyl-(1→2)-β-D-glucopyranoside (45)

The overall reaction scheme is as follows:

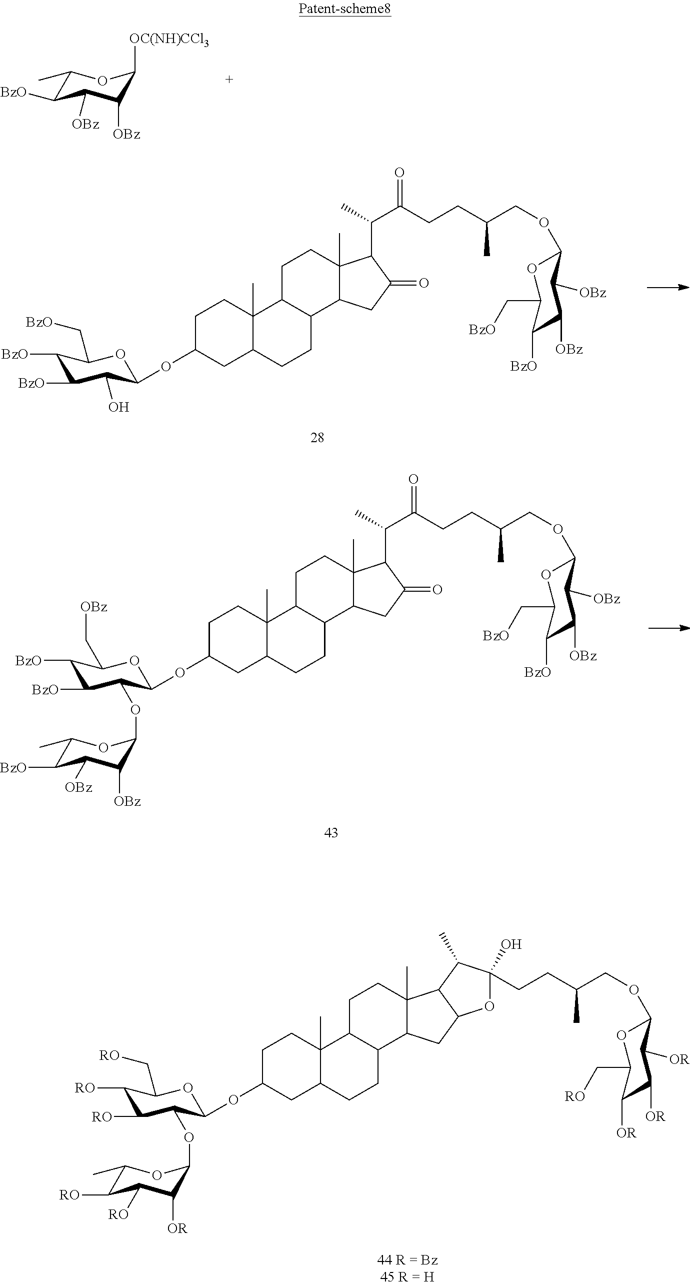

### Preparation of (43)

To a mixture of compound 42 (1.5 g, 2.4 mmol, commercially available) and 28 (3.27 g, 2.2 mmol) in anhyd CH₂Cl₂ (50 mL) at −20° C. was added TMSOTf (44 μL, 0.24 mmol). The mixture was stirred at these conditions for 40 mm, then neutralized with TEA, and concentrated. The residue was purified by column chromatography to give a foamy solid 43 (3.68 g, 86%). MALDITOF-MS: Calcd for C₁₁₅H₁₁₄O₂₈: 1942.75 [M]⁺. Found 1965.60 [M Na]⁺.

### Preparation of (44)

The mixture of compound 43 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 44 as a white solid (115 mg, 70%). MALDITOF-MS: Calcd for C₁₁₅H₁₁₆O₂₈: 1944.75 [M]⁺. Found 1967.60 [M Na]⁺.

### Preparation of (45)

Compound 44 (100 mg, 0.051 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 45 as an amorphous solid (44 mg, 95%): ¹H NMR (400 MHz, C₆D₅N): δ5.49 (d, 1H, J 3.5 Hz, H-1), 5.25 (d, 1H, J 7.5 Hz, H-1), 5.09 (d, 1H, J 7.9 Hz, H-1), ¹³C NMR (100 MHz, CD₅N): δ 106.8, 104.0, 102.1. MALDITOF-MS: Calcd for C₄₅H₇₆O₁₈: 904.50 [M]⁺. Found 947.80 [M+Na]⁺.

### Example 9

### The Preparation of (25S)-26-β-D-glucopyranosyl-22-hydroxy-5β-furostane-3β,26-diol-3-O-α-L-rhamnopyranosyl-(1→4)-β-D-glucopyranoside (49)

The overall reaction scheme is as follows:

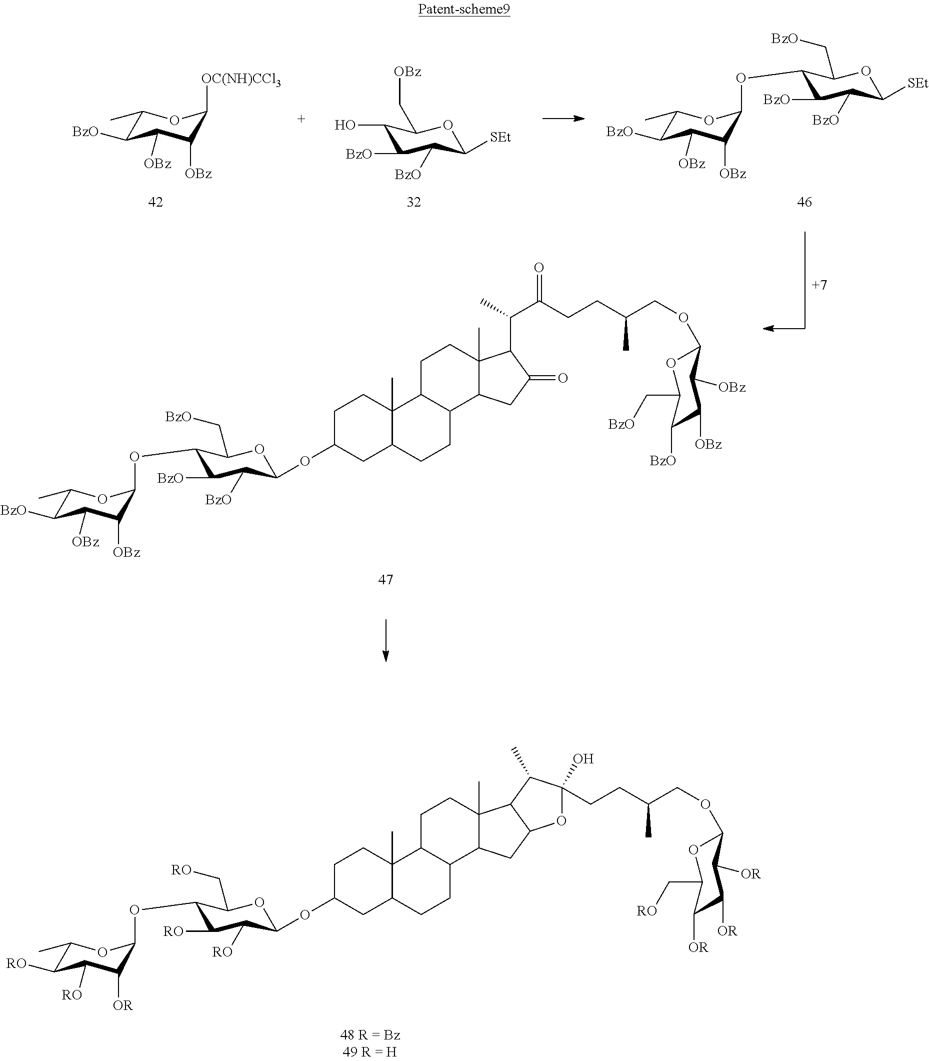

### Preparation of (47)

To a mixture of compound 32 (2.1 g, 3.9 mmol, commercially available) and 42 (2.85 g, 4.6 mmol) in anhyd CH₂Cl₂ (45 mL) at −20° C. was added TMSOTf (83 μL, 0.46 mmol). The mixture was stirred at these conditions for 30 min, then neutralized with TEA, and concentrated. The residue was purified by column chromatography to give a foamy solid 46 (3.57 g, 92%). To a mixture of 46 (2.1 g, 2.11 mmol) and 7 (1.94 g, 1.92 mmol) in anhyd CH₂Cl₂ (45 mL) at −20° C. was added MS (712 mg, 3.2 mmol) and Me₃SiOTf (38 μL, 0.211 mmol) under N₂, atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 47 as a foamy solid (3.17 g, 85%). MALDITOF-MS: Calcd for C₁₁₅H₁₁₄O₂₈: 1942.75 [M]⁺. Found 1965.60 [M+Na]⁺.

### Preparation of (48)

The mixture of compound 47 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 48 as a white solid (115 mg, 70%). MALDITOF-MS: Calcd for C₁₁₅H₁₁₆O₂₈: 1944.75 [M]⁺. Found 1967.60 [M+Na]⁺.

### Preparation of (49)

Compound 48 (100 mg, 0.051 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 49 as an amorphous solid (44 mg, 95%). MALDITOF-MS: Calcd for C₄₅H₇₆O₁₈: 904.50 [M]⁺. Found 947.80 [M+Na]⁺.

### Example 10

### The Preparation of (25S)-26-O-β-D-glucopyranosyl-22-hydroxy-5β-furostane-3β,26-diol-3-O-α-L-rhamnopyranosyl-(1→6)-β-D-galactopyranoside (54)

The overall reaction scheme is as follows;

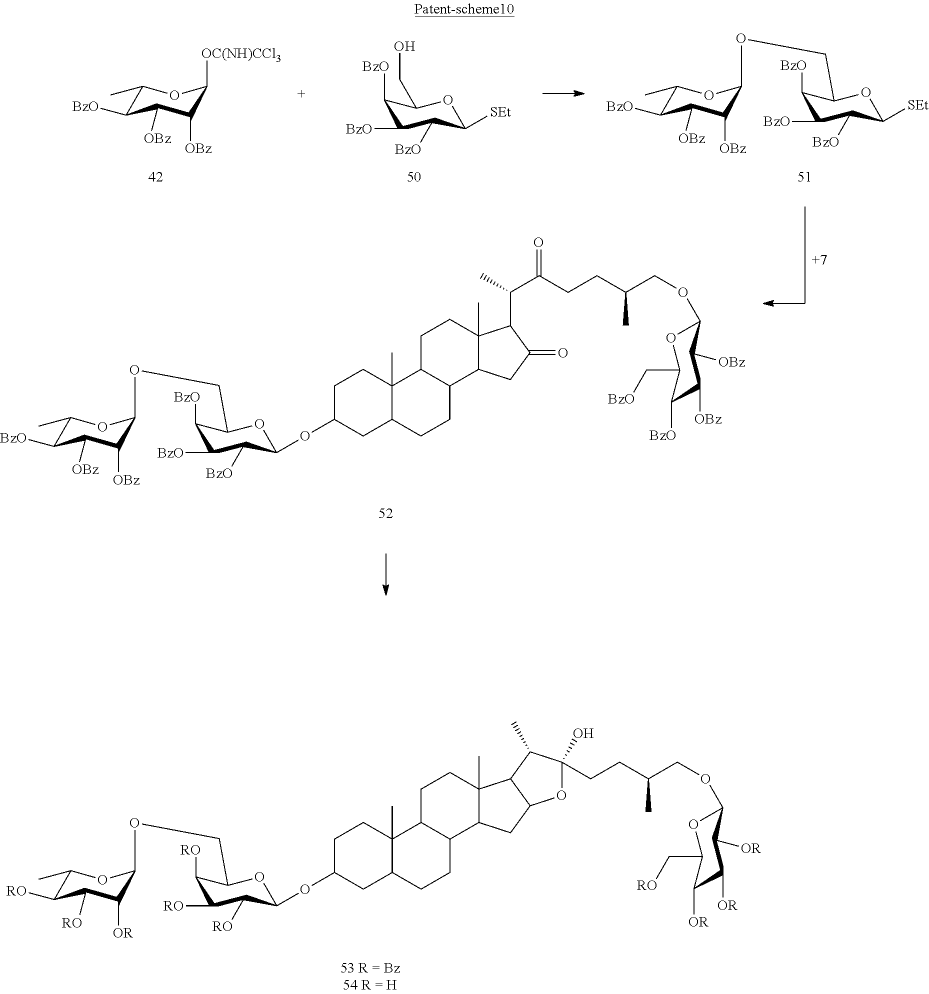

### Preparation of (52)

To a mixture of compound 50 (2.1 g, 3.9 mmol, commercially available from Beijing Carbomex Biotech Co. Ltd.) and 42 (2.85 g, 4.6 mmol) in anhyd CH₂Cl₂ (50 mL) at −20° C. was added TMSOTf (83 μL, 0.46 mmol). The mixture was stirred at these conditions for 1 h, then neutralized with TEA, and concentrated. The residue was purified by column chromatography to give a foamy solid 51 (3.69 g, 95%). To a mixture of 51 (2.1 g, 2.11 mmol) and 7 (1.94 g, 1.92 mmol) in anhyd CH₂Cl₂ (40 mL) at −20° C. was added NIS (712 mg, 3.2 mmol) and Me₃SiOTf (38 μL, 0.211 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated, Column chromatography (petroleum ether-EtOAc, 2:1) of the residue gave 52 as a foamy solid (3.17 g, 85%). MALDITOF-MS: Calcd for C₁₁₅H₁₁₄O₂₈: 1942.75 [M]⁺. Found 1965.60 [M+Na]⁺.

### Preparation of (53)

The mixture of compound 52 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 53 as a white solid (115 mg, 68%). MALDITOF-MS: Calcd for C₁₁₅H₁₁₆O₂₈: 1944.75 [M]⁺. Found 1967.60 [M+Na]⁺.

### Preparation of (54)

Compound 53 (100 mg, 0.057 mmol) was dissolved in anhyd CH₂Cl₂-MeOH (1:2, 18 mL), and then 1.0 M NaOMe in MeOH (0.2 mL) was added at 0° C., After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 54 as an amorphous solid (44 mg, 95%): ¹H NMR (400 MHz, C₆D₅N): δ5.37 (d, 1H, J 2.5 Hz, H-1), 5.24 (d, 1H, J 7.5 Hz, H-1), 5.18 (d, 1H, J 7.6 Hz, H-1). ¹³C NMR (100 MHz, CD₅N): δ 105.5, 104.1, 102.5. MALDITOF-MS: Calcd for C₄₅H₇₆O₁₈: 904.50 [M]⁺. Found 947.80 [M+Na]⁺.

### Example 11

### The Preparation of (25S)-26-O-β-D-glucopyranosyl-22-hydroxy-5β-furostane-3β,26-diol-3-O-[α-L-rhamnopyranosyl-(1→2)]-[α-L-rhamnopyranosyl-(1→6)]-β-D-glucopyranoside (59)

The overall reaction scheme is as follows:

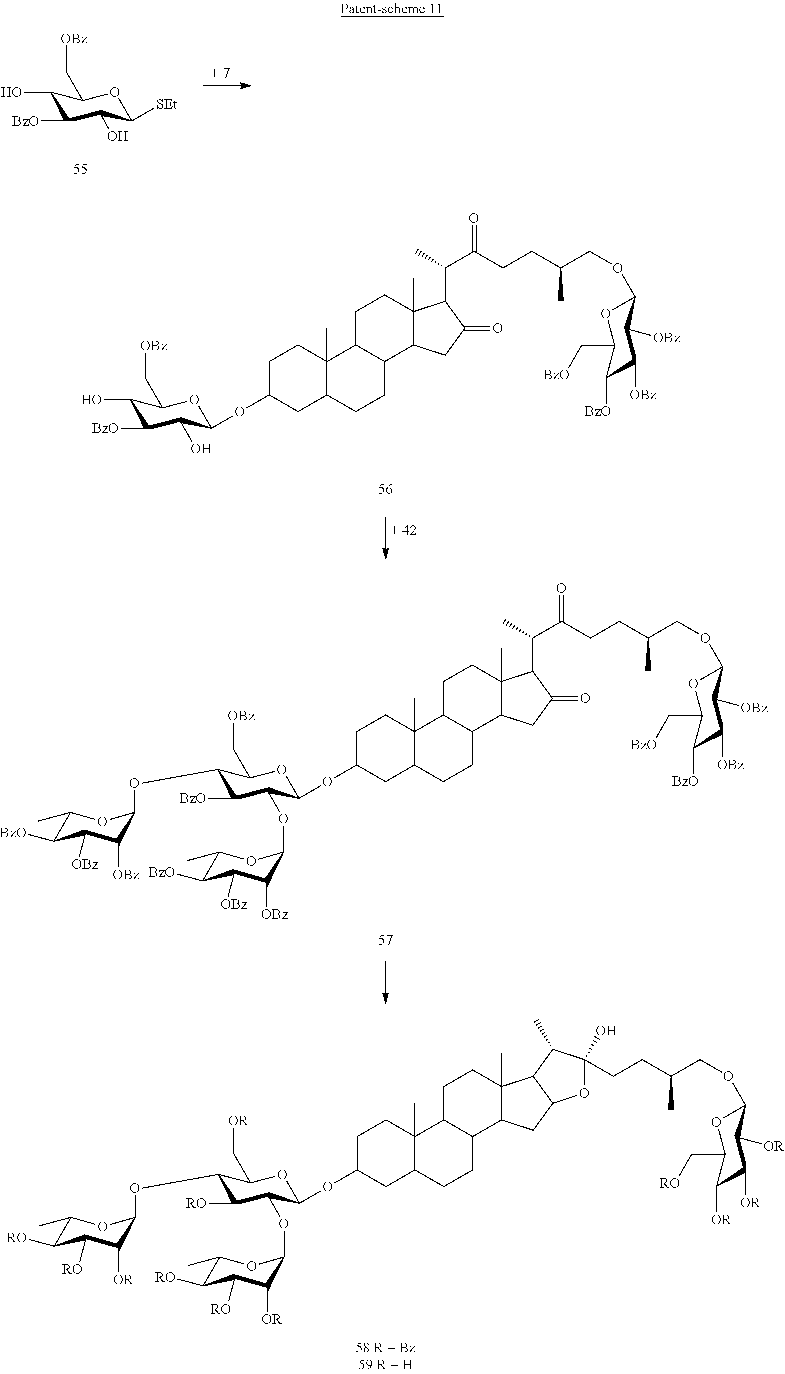

### Preparation of (56)

To a mixture of 55 (996 mg, 2.30 mmol) and 7 (1.94 g, 1.92 mmol) in anhyd CH₂Cl₂ (20 mL) at −40° C. was added NIS (517 mg, 2.30 mmol) and Me₃SiOTf (42 μL, 0.23 mmol) under N₂ atmosphere at −20° C. The mixture was stirred under these conditions for 40 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 56 as a foamy solid (2.12 g, 80%). MALDITOF-MS: Calcd for C₈₁H₈₈O₂₀: 1380.59 [M]⁺. Found 1403.70 [M+Na]⁺.

### Preparation of (57)

To a mixture of 56 (2.0 g, 1.45 mmol) and 42 (2.16 g, 3.48 mmol) in anhyd CH₂Cl₂ (30 mL) at −20° C. was added Me₃SiOTf (62 μL, 0.34 mmol) under N₂ atmosphere at −20° C. The mixture was stirred under these conditions for 30 min, at the end of which time TLC (petroleum ether-EtOAc, 3:1) indicated that all starting materials were consumed. The reaction mixture was neutralized with TEA, then concentrated. Column chromatography (petroleum ether-EtOAc, 2:1) of the residue gave 57 as a foamy solid (2.83 g, 85%). MALDITOF-MS: Calcd for C₁₃₅H₁₃₂O₃₄: 2296.86 [M]⁺. Found 2319.90 [M+Na]⁺.

### Preparation of (58)

The mixture of compound 57 (195 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 58 as a white solid (121 mg, 68%). MALDITOF-MS: Calcd for C₁₃₅H₁₃₄O₃₄: 2298.86 [M]⁺. Found 2321.90 [M+Na]⁺.

### Preparation of (59)

Compound 58 (120 mg, 0.052 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:1) indicated that the reaction was complete. The solution was neutralized with ion-exchange resin (H⁺), and then filtered and concentrated. The residue was purified by Bio-gel P₂ column to afford 59 as an amorphous solid (53 mg, 97%): Selected ¹H NMR (400 MHz, C₆D₅N): δ5.46 (d, 1H, J 2.5 Hz, H-1), 5.41 (d, 1H, J 2.4 Hz, H-1), 5.09 (d, 1H, J 7.5 Hz, H-1), 5.00 (d, 1H, J 7.6 Hz, H-1). ¹³C NMR (100 MHz, CD₅N): δ 108.5, 105.1, 102.9, 102.3. MALDITOF-MS: Calcd for C₅₁H₈₆O₂₂: 1050.56 [M]⁺. Found 1073.80 [M+Na]⁺.

### Example 12

### The Preparation of (25S)-26-O-β-D-glucopyranosyl-22-hydroxy-5β-furostane-3β,26-diol-3-O-[β-D-glucopyranosyl-(1→4)]-[α-L-rhamnopyranosyl-(1→2)]-β-D-glucopyranoside (62)

The overall reaction scheme is as follows:

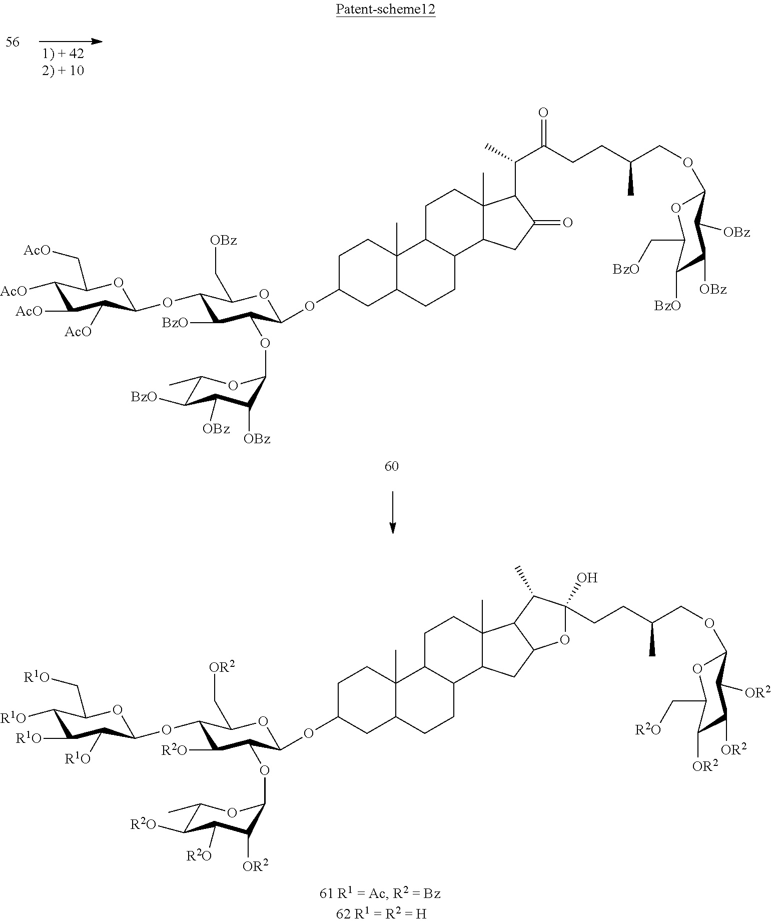

### Preparation of (60)

To a mixture of compound 56 (2.5 g, 1.81 mmol) and 42 (1.35 g, 2.17 mmol) in anhyd CH₂Cl₂ (50 mL) at −40° C. was added TMSOTf (40 μL, 0.22 mmol). The mixture was stirred at these conditions for 40 min, then the mixture was added 10 (1.07 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 60 as a foamy solid (3.10 g, 79%). MALDITOF-MS: Calcd for C₁₂₂H₁₂₈O₃₆: 2168.82 [M]⁺. Found 2192.0 [M+Na]⁺.

### Preparation of (61)

The mixture of compound 60 (185 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 61 as a white solid (118 mg, 64%). MALDITOF-MS: Calcd for C₁₂₂H₁₃₀O₃₆: 2170.82 [M]⁺. Found 2194.0 [M+Na]⁺.

### Preparation of (62)

Compound 61 (100 mg, 0.046 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 62 as an amorphous solid (47 mg, 95%). MALDITOF-MS: Calcd for C₅₁H₈₆O₂₃: 1066.56 [M]⁺. Found 1089.72 [M+Na]⁺.

### Example 13

### The Preparation of (25S)-26-O-β-D-glucopyranosyl-22-hydroxy-5β-furostane-3β,26-diol-3-O-[α-L-rhamnopyranosyl-(1→4)]-[β-D-glucopyranosyl-(1→2)]-β-D-glucopyranoside (65)

The overall reaction scheme is as follows:

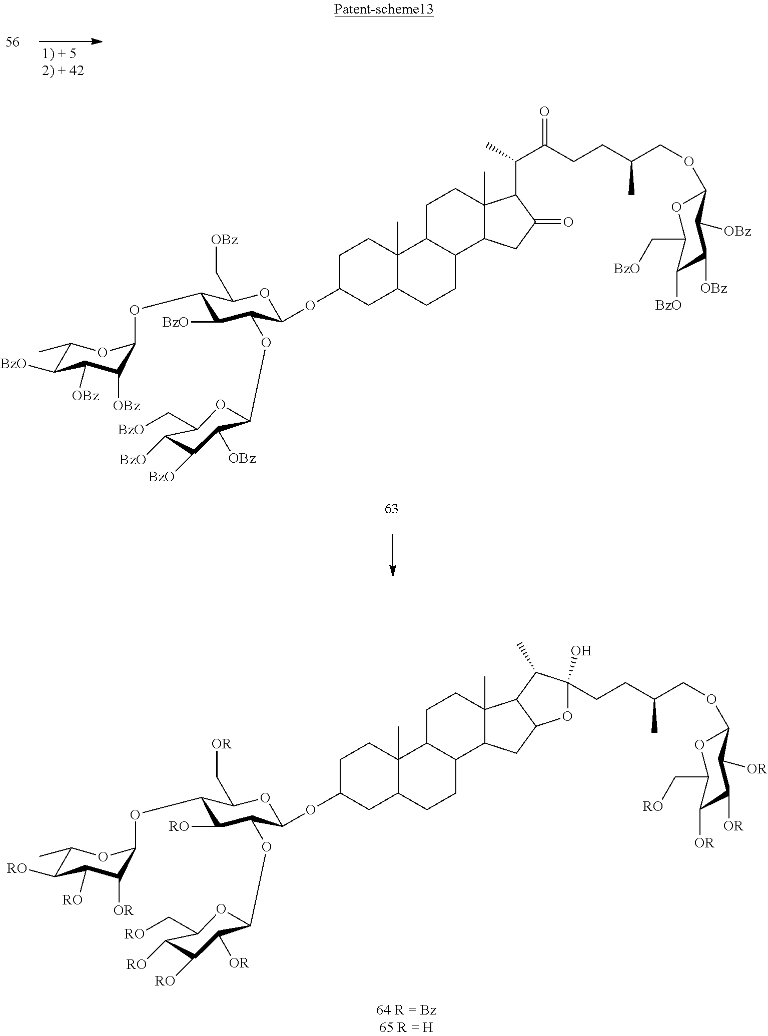

### Preparation of (63)

To a mixture of compound 56 (2.5 g, 1.81 mmol) and 5 (1.61 g, 2.17 mmol) in anhyd CH₂Cl₂ (50 mL) at −40° C. was added TMSOTf (40 μL, 0.22 mmol). The mixture was stirred at these conditions for 40 min, then the mixture was added 42 (1.35 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 63 as a foamy solid (3.46 g, 79%). MALDITOF-MS: Calcd for C₁₄₂H₁₃₆O₃₆: 2416.88 [M]⁺. Found 2439.75 [M+Na]⁺.

### Preparation of (64)

The mixture of compound 63 (206 mg, 0.085 mmol) and NaBH₄ (96 mg, 2.5 mmol) in 2-propanol (8 mL) and CH₂C17 (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 64 as a white solid (123 mg, 60%). MALDITOF-MS: Calcd for C₁₄₂H₁₃₈O₃₆: 2418.88 [M]⁺; Found 2441.75 [M+Na]⁺.

### Preparation of (65)

Compound 64 (120 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 65 as an amorphous solid (51 mg, 95%). MALDITOF-MS: Calcd for C₅₁H₈₆O₂₃: 1066.56 [M]⁺. Found 1089.72 [M+Na]⁺.

### Example 14

### The Preparation of (25S)-26-O-β-D-glucopyranosyl-22-hydroxy-5β-furostane-3β,26-diol-3-O-[β-D-glucopyranosyl-(1→4)]-[α-L-rhamnopyranosyl-(1→2)]-β-D-galactopyranoside (68)

The overall reaction scheme is as follows:

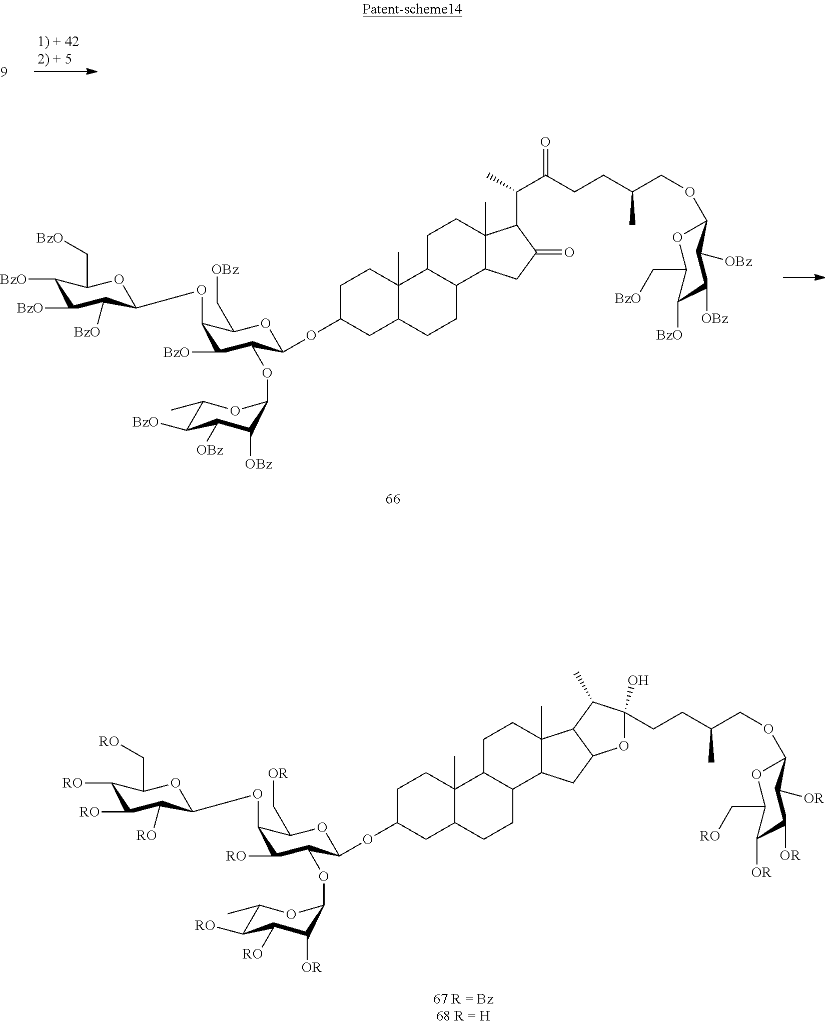

### Preparation of (66)

To a mixture of compound 9 (2.5 g, 1.81 mmol) and 42 (1.35 g, 2.17 mmol) in anhyd CH₂Cl₂ (50 mL) at −40° C. was added TMSOTf (40 μL, 0.22 mmol). The mixture was stirred at these conditions for 40 min, then the mixture was added 5 (1.61 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 66 as a foamy solid (3.46 g, 79%). MALDITOF-MS: Calcd for C₁₄₂H₁₃₆O₃₆: 2416.88 [M]⁺. Found 2439.75 [M+Na]⁺.

### Preparation of (67)

The mixture of compound 66 (206 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 67 as a white solid (123 mg, 60%). MALDITOF-MS: Calcd for C₁₄₂H₁₃₈O₃₆: 2418.88 [M]⁺. Found 2441.75 [M+Na]⁺.

### Preparation of (68)

Compound 67 (120 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 66 as an amorphous solid (49 mg, 93%): Selected ¹H NMR (400 MHz, C₆D₅N): δ5.50 (d, 1H, J 2.7 Hz, H-1), 5.37 (d, 1H, J 7.2 Hz, H-1), 5.04 (d, 1H, J 7.3 Hz, H-1), 5.00 (d, 1H, J 7.9 Hz, H-1). ¹³C NMR (100 MHz, CD₅N): δ 107.5, 105.1, 102.9, 102.1. MALDITOF-MS: Calcd for C₅₁H₈₆O₂₃: 1066.56 [M]⁺. Found 1089.72 [M+Na]⁺.

### Example 15

### The Preparation of (25S)-26-O-β-D-glucopyranosyl-22-hydroxy-5β-furostane-3β,26-diol-3-O-[α-L-rhamnopyranosyl-(1→4)]-[β-D-glucopyranosyl-(1→2)]-β-D-galactopyranoside (71)

The overall reaction scheme is as follows:

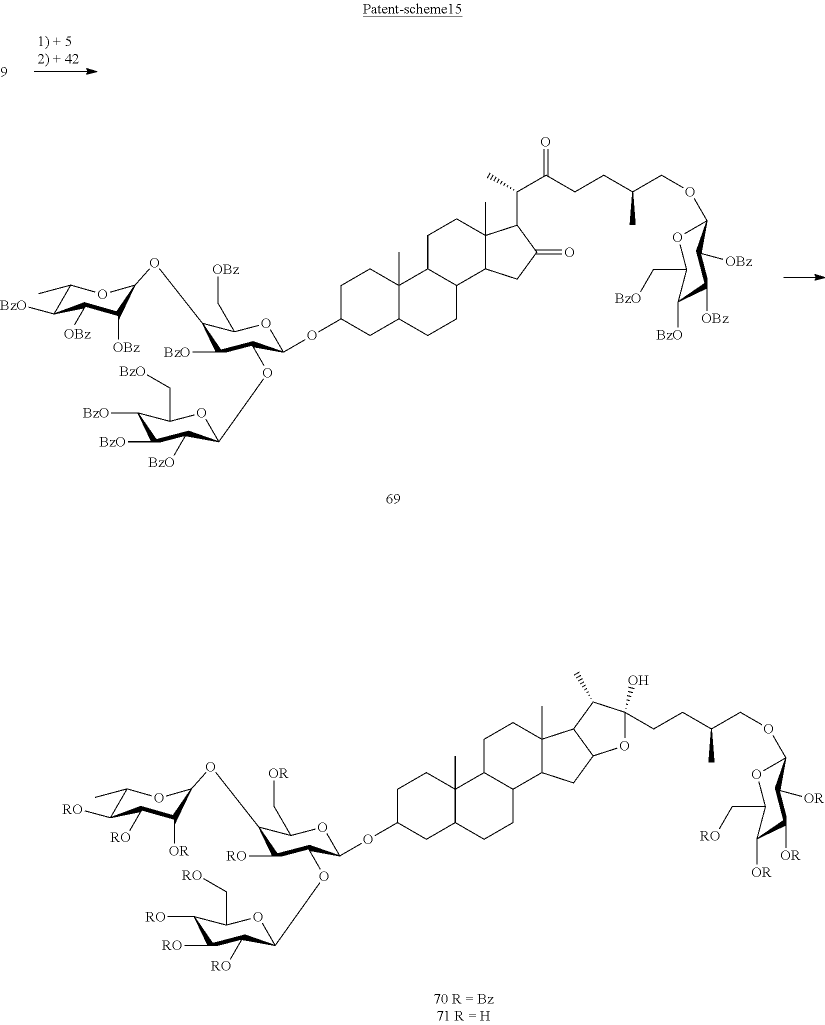

### Preparation of (69)

To a mixture of compound 9 (2.5 g, 1.81 mmol) and 5 (1.61 g, 2.17 mmol) in anhyd CH₂Cl₂ (50 mL) at −40° C. was added TMSOTf (40 μL, 0.22 mmol). The mixture was stirred at these conditions for 40 min, then the mixture was added 42 (1.35 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 69 as a foamy solid (3.46 g, 79%). MALDITOF-MS: Calcd for C₁₄₂H₁₃₆O₃₆: 2416.88 [M]⁺. Found 2439.75 [M+Na]⁺.

### Preparation of (70)

The mixture of compound 69 (206 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 70 as a white solid (123 mg, 60%). MALDITOF-MS: Calcd for C₁₄₂H₁₃₈O₃₆: 2418.88 [M]⁺. Found 2441.75 [M+Na]⁺.

### Preparation of (71)

Compound 70 (120 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 71 as an amorphous solid (49 mg, 93%): Selected ¹H NMR (400 MHz, C₆D₅N): δ5.51 (d, 1H, J 2.7 Hz, H-1), 5.32 (d, 1H, J 7.2 Hz, H-1), 5.05 (d, 1H, J 7.3 Hz, H-1), 5.02 (d, 1H, J 7.9 Hz, H-1). ¹³C NMR (100 MHz; CD₅N): δ 107.5, 104.0, 103.2, 102.0. MALDITOF-MS: Calcd for C₅₁H₈₆O₂₃: 1066.56 [M]⁺. Found 1089.72 [M+Na]⁺.

### Example 16

### The Preparation of (25S)-26-O-β-D-glucopyranosyl-22-hydroxy-5β-furostane-3β3,26-diol-3-O-β-D-glucopyranosyl-(1→4)-β-D-glucopyranosyl-(1→4)-β-D-galactopyranoside (76)

The overall reaction scheme is as follows:

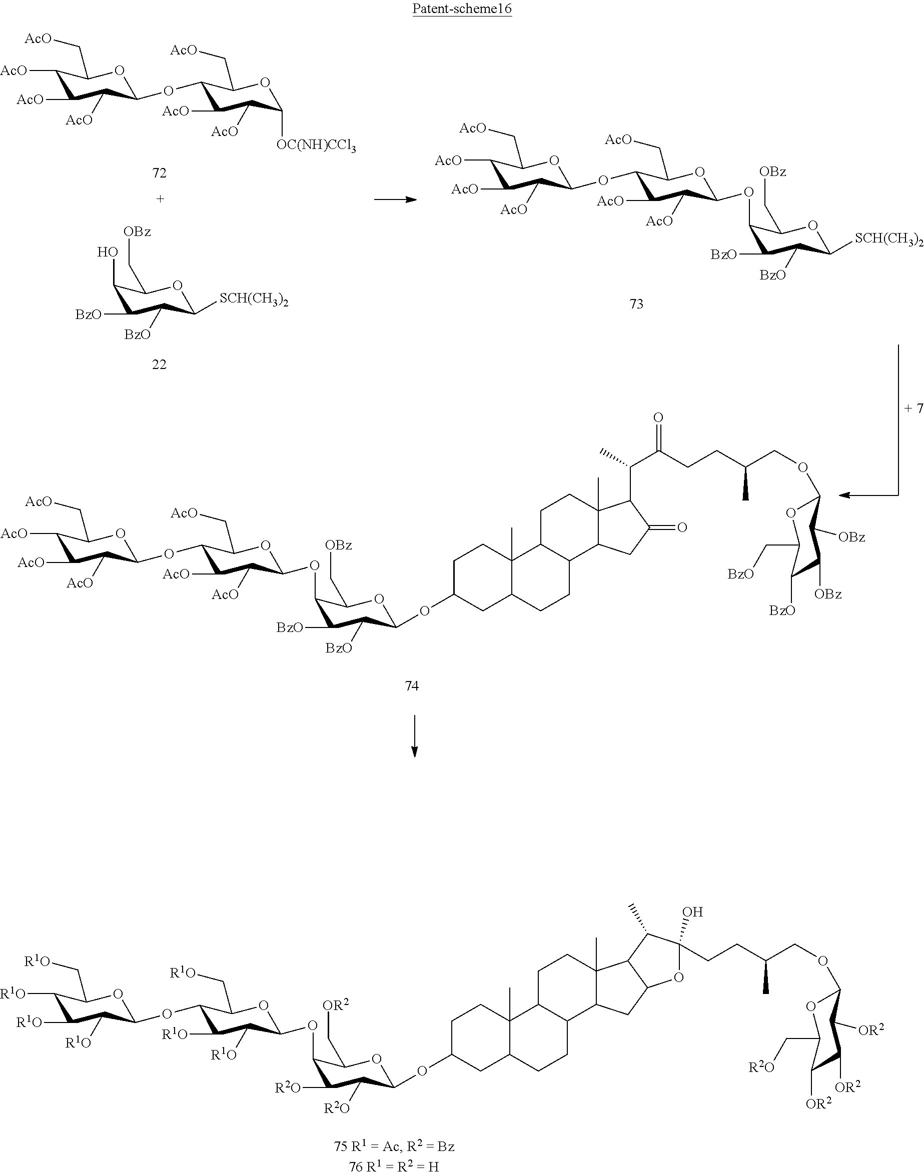

### Preparation of (74)

To a mixture of compound 72 (2.5 g, 3.20 mmol) and 22 (1.60 g, 2.91 mmol) in anhyd CH₂Cl₂ (50 mL) at −20° C. was added TMSOTf (58 μL, 0.32 mmol). The mixture was stirred at these conditions for 40 min, then neutralized with TEA, and concentrated. The residue was purified by column chromatography to give a foamy solid 73 (193 g, 86%). To a mixture of 73 (2.0 g, 1.7 mmol) and 7 (1.57 g, 1.55 mmol) in anhyd CH₂Cl₂ (50 mL) at −20° C. was added NIS (562 mg, 2.5 mmol) and Me₃SiOTf (31 μL, 0.17 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 74 as a foamy solid (2.71 g, 83%). MALDITOF-MS: Calcd for C₁₁₄H₁₂₆O₃₈: 2102.79 [M]⁺. Found 2125.90 [M+Na]⁺.

### Preparation of (75)

The mixture of compound 74 (179 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₅SO₄, and the solvent was removed under vacuum to give an colorless oil, which was purified by column chromatography (petroleum ether/ethyl acetate, 1:1) to give compound 75 as a white solid (116 mg, 67%). MALDITOF-MS: Calcd for C₁₁₄H₁₂₈O₃₈: 2104.79 [M]⁺. Found 2127.90 [M+Na]⁺.

### Preparation of (76)

Compound 75 (100 mg, 0.047 mmol) was dissolved in anhyd CH₂Cl₂-MeOH (1:2, 18 mL), and then 1.0 M NaOMe in MeOH (0.2 mL) was added at 0° C. After stirring at room temperature for S h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 76 as an amorphous solid (49 mg, 96%): Selected ¹H NMR (400 MHz, C₆D₅N): δ5.35 (d, 1H, J 7.2 Hz, H-1), 5.31 (d, 1H, J 7.2 Hz, H-1), 5.10 (d, 1H, J 7.3 Hz, H-1), 5.00 (d, 1H, J 8.0 Hz, H-1). ¹³C NMR (100 MHz, CD₅N): δ 105.1, 103.4, 102.9, 101.3. MALDITOF-MS: Calcd for C₅₁H₈₆O₂₄: 1082.55 [M]⁺. Found 1105.70 [M+Na]⁺.

### Example 17

### The Preparation of (25S)-26-O-β-D-glucopyranosyl-22-hydroxy-5β-furostane-3β,26-diol-3-O-β-D-glucopyranosyl-(1→4)-β-D-glucopyranosyl-(1→2)-β-D-galactopyranoside (79)

The overall reaction scheme is as follows:

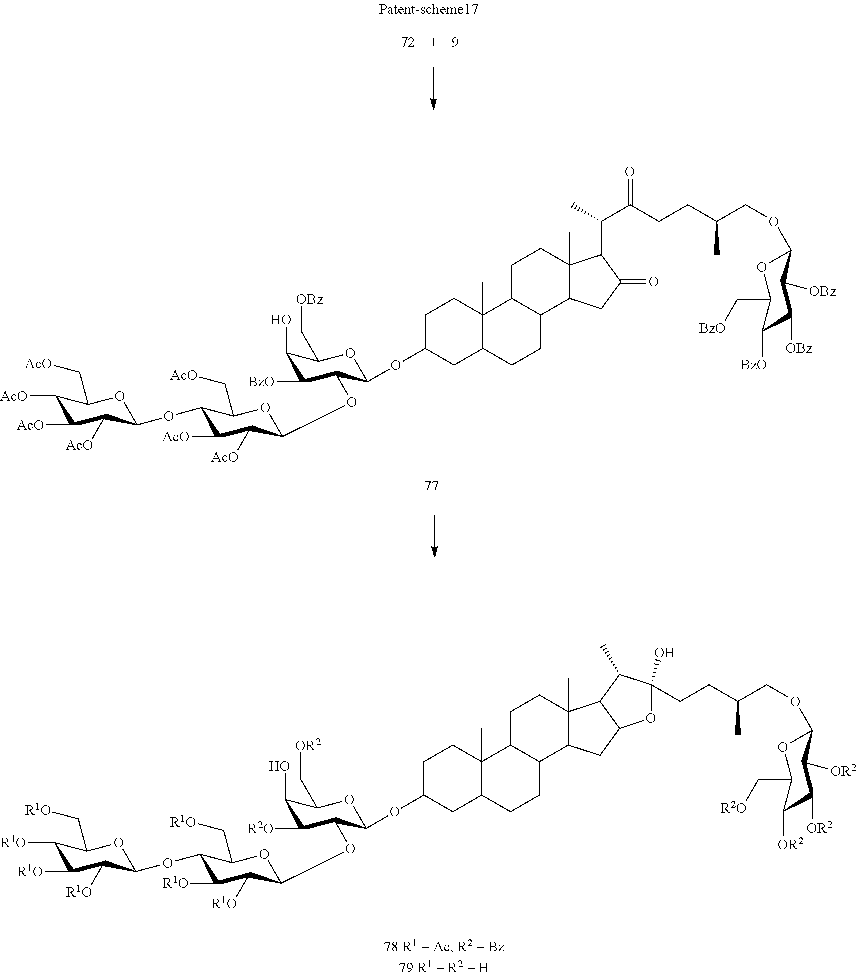

### Preparation of (77)

To a mixture of compound 72 (2.5 g, 3.20 mmol) and 9 (4.02 g, 2.91 mmol) in anhyd CH₂Cl₂ (50 mL) at −40° C. was added TMSOTf (58 μL, 0.32 mmol). The mixture was stirred at these conditions for 1 h, then neutralized with TEA, and concentrated. The residue was purified by column chromatography to give a foamy solid 77 (4.54 g, 78%), MALDITOF-MS: Calcd for C₁₀₇H₁₂₂O₃₇: 1998.77 [M]⁺. Found 2021.90 [M+Na]⁺.

### Preparation of (78)

The mixture of compound 77 (170 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 78 as a white solid (116 mg, 68%). MALDITOF-MS: Calcd for C₁₀₇H₁₂₄O₃₇: 2000.77 [M]⁺. Found 2023.90 [M+Na]⁺.

### Preparation of (79)

Compound 78 (100 mg, 0.05 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 79 as an amorphous solid (51 mg, 94%): Selected ¹H NMR (400 MHz, C₆D₅N): δ5.34 (d, 1H, J 7.5 Hz, H-1), 5.18 (d, 1H, J 7.2 Hz, H-1), 5.08 (d, 1H, J 7.3 Hz, H-1), 5.05 (d, 1H, 7.9 Hz, H-1). ¹³C NMR (100 MHz, CD₅N): δ 105.5, 103.1, 102.9, 101.9. MALDITOF-MS: Calcd for C₅₁H₈₆O₂₄: 1082.55 [M]⁺. Found 1105.70 [M+Na]⁺.

### Example 18

### The Preparation of (25S)-26-O-β-D-glucopyranosyl-22-hydroxy-5β-furostane-3β,26-diol-3-O-β-D-glucopyranosyl-(1→4)-β-D-glucopyranosyl-(1→4)-β-D-glucopyranoside (83)

The overall reaction scheme is as follows:

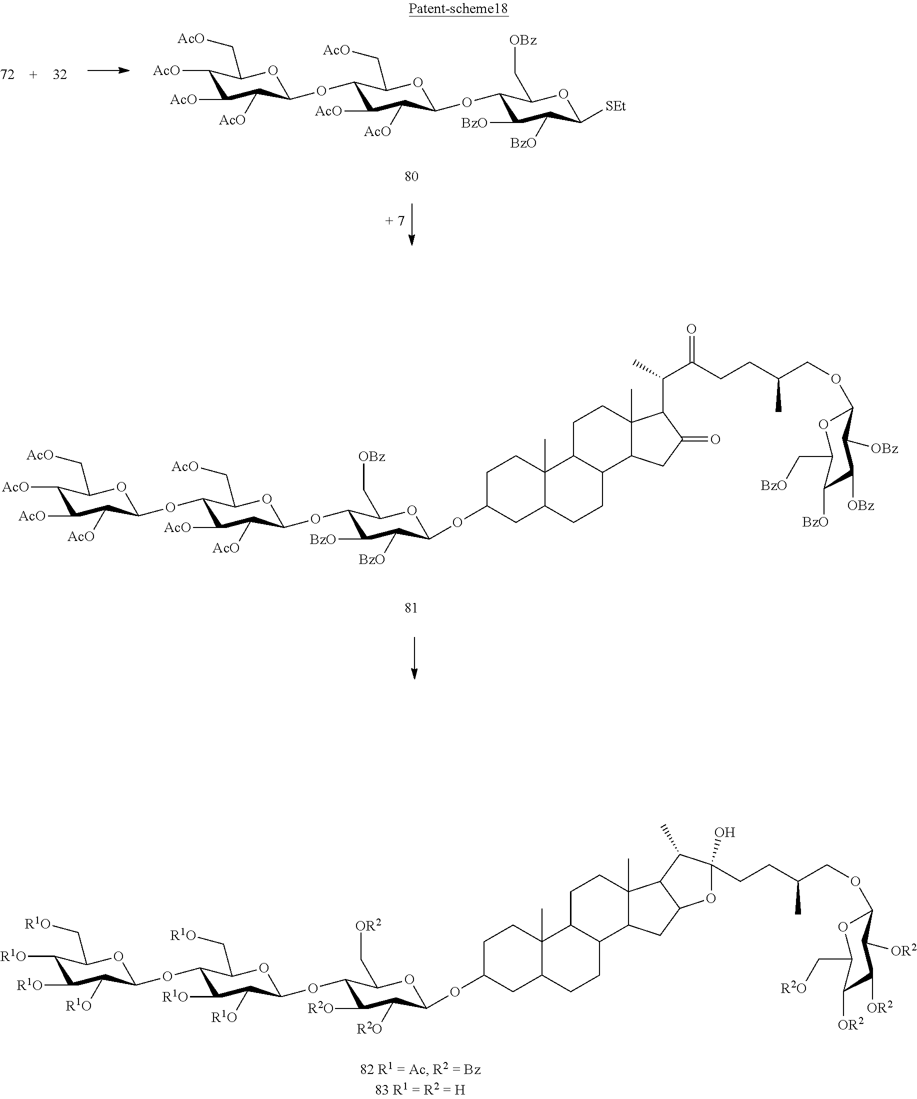

### Preparation of (81)

To a mixture of compound 32 (2.1 g, 3.9 mmol) and 72 (3.6 g, 4.6 mmol) in anhyd CH₂Cl₂ (50 mL) at −20° C. was added TMSOTf (83 μL, 0.46 mmol). The mixture was stirred at these conditions for 30 min, then neutralized with TEA, and concentrated. The residue was purified by column chromatography to give a foamy solid 80 (4.14 g, 92%). To a mixture of 80 (2.5 g, 2.16 mmol) and 7 (1.98 g, 1.96 mmol) in anhyd CH₂Cl₂ (50 mL) at −20° C. was added NIS (729 mg, 3.24 mmol) and Me₃SiOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 1:1) of the residue gave 81 as a foamy solid (3.51 g, 85%). MALDITOF-MS: Calcd for C₁₁₄H₁₂₆O₃₈: 2102.79 [M]⁺. Found 2125.90 [M+Na]⁺.

### Preparation of (82)

The mixture of compound 81 (179 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 82 as a white solid (116 mg, 67%). MALDITOF-MS: Calcd for C₁₁₄H₁₂₈O₃₈: 2104.79 [M]⁺. Found 2127.90 [M+Na]⁺.

### Preparation of (83)

Compound 82 (100 mg, 0.047 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 83 as an amorphous solid (49 mg, 96%): Selected ¹H NMR (400 MHz, C₆D₅N): δ5.29 (d, 1H, J 7.5 Hz, H-1), 5.20 (d, 1H, J 7.2 Hz, H-1), 5.11 (d, 1H, J 7.5 Hz, H-1), 5.05 (d, 1H, J 8.0 Hz, H-1). ¹³C NMR (100 MHz, CD₆N): δ 104.3, 103.0: 102.9, 101.0. MALDITOF-MS: Calcd for C₅₁H₈₆O₂₄: 1082.55 [M]⁺. Found 1105.70 [M+Na]⁺.

### Example 19

### The Preparation of (25S)-26-O-β-D-glucopyranosyl-22-hydroxy-5β-furostane-3β,26-diol-3-O-β-D-glucopyranosyl-(1→4)-β-D-glucopyranosyl-(1→2)-β-D-glucopyranoside (86)

The overall reaction scheme is as follows:

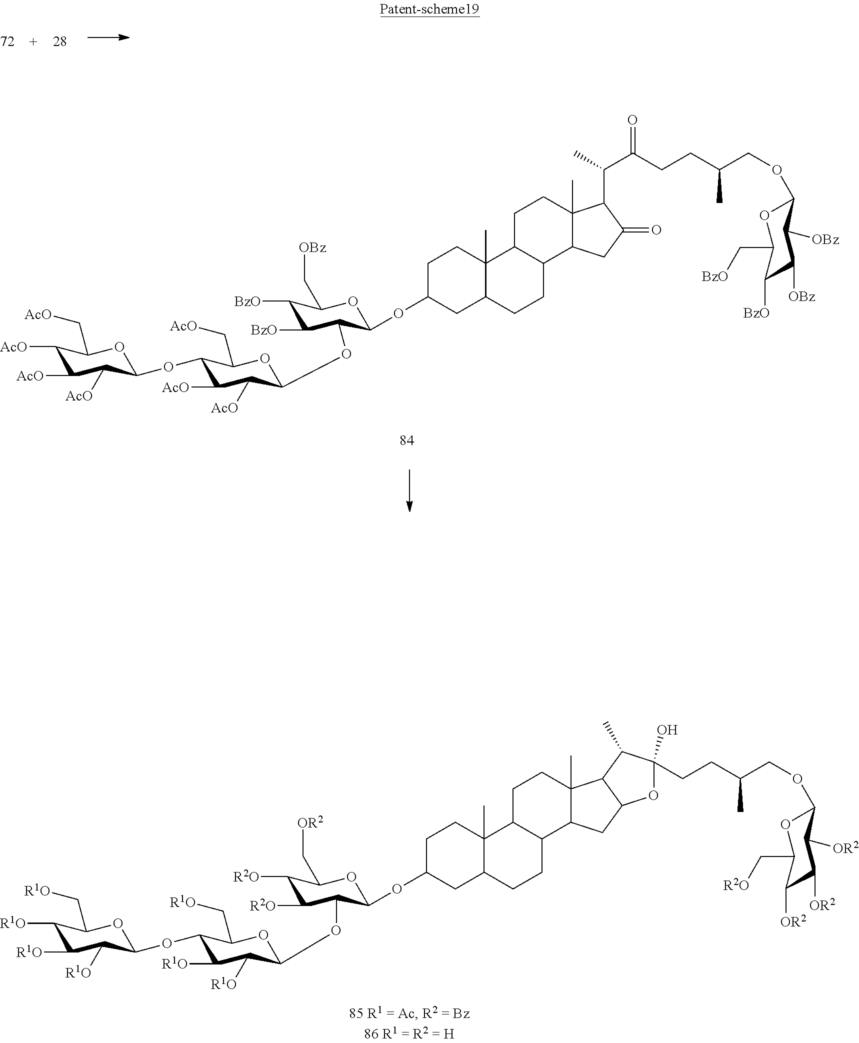

### Preparation of (84)

To a mixture of compound 28 (5.8 g, 3.9 mmol) and 72 (3.6 g, 4.6 mmol) in anhyd CH₂Cl₂ (55 mL) at −20° C. was added TMSOTf (83 μL, 0.46 mmol). The mixture was stirred at these conditions for 30 min, then neutralized with TEA, and concentrated. The residue was purified by column chromatography to give a foamy solid 84 (4.14 g, 92%). (6.97 g, 85%). MALDITOF-MS: Calcd for C₁₁₄H₁₂₆O₃₈: 2102.79 [M]⁺. Found 2125.90 [M+Na]⁺.

### Preparation of (85)

The mixture of compound 84 (179 mg, 0.085 mmol) and NaBH₄ (96 mg, 2.5 mmol) in 2-propanol (8 mL) and C₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 85 as a white solid (116 mg, 67%). MALDITOF-MS: Calcd for C₁₁₄H₁₂₈O₃₈: 2104.79 [M]⁺. Found 2127.90 [M Na]⁺.

### Preparation of (86)

Compound 85 (100 mg, 0.047 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 86 as an amorphous solid (50 mg, 96%): Selected ¹H NMR (400 MHz, C₆D₅N): δ5.24 (d, 1H, J 7.5 Hz, H-1), 5.11 (d, 1H, J 7.2 Hz, H-1), 5.10 (d, 1H, J 7.7 Hz, H-1), 5.09 (d, 1H, J 7.9 Hz, H-1). ¹³C NMR (100 MHz, CD₅N): δ 103.5, 103.3, 102.9, 101.0. MALDITOF-MS: Calcd for C₅₁H₈₆O₂₄: 1082.55 [M]⁺. Found 1105.70 [M+Na]⁺

### Example 20

### The Preparation of (25S)-26-β-D-galactopyranosyl-22-hydroxy-5β-furostane-3β,26-diol-3-O-β-D-galactopyranoside (92)

The overall reaction scheme is as follows:

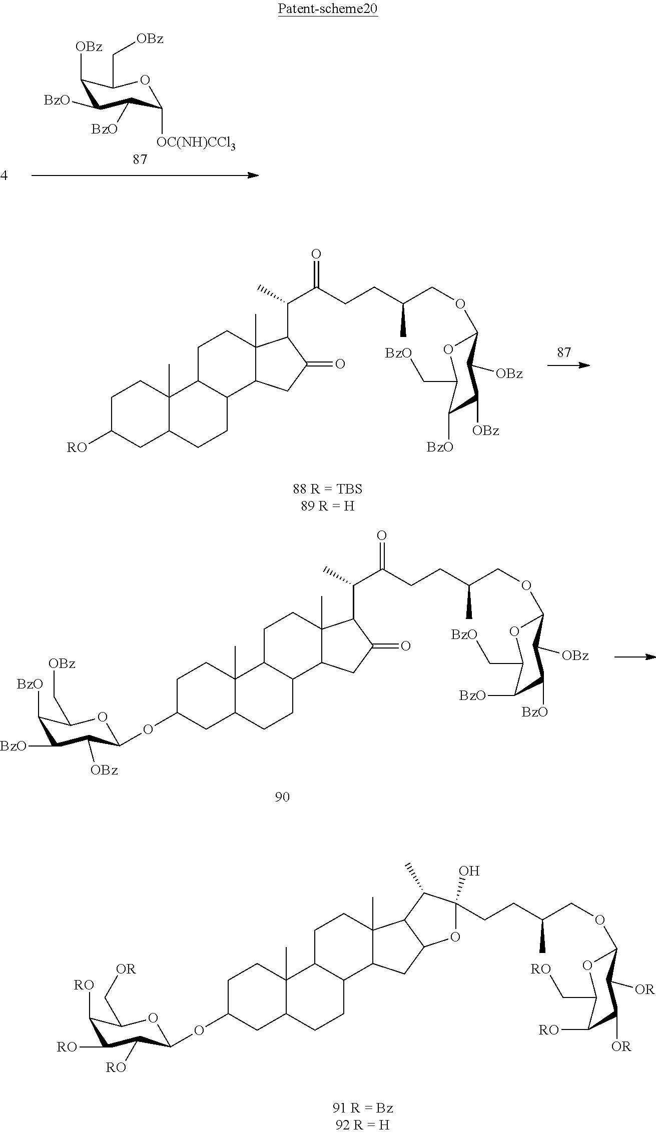

### Preparation of (89)

To a mixture of compounds 4 (2.25 g, 4.1 mmol) and 87 (3.56 g, 4.8 mmol, commercially available) in anhyd CH₂Cl₂ (36 mL) was added Me₃SiOTf (86 μL, 0.47 mmol) under N₂ atmosphere at −20° C. The mixture was stirred under these conditions for 40 min, at the end of which time TLC (petroleum ether-EtOAc, 4:1) indicated that all starting materials were consumed. The reaction mixture was neutralized with triethylamine (TEA), then concentrated. Column chromatography (petroleum ether-EtOAc, 6:1) of the residue gave 88 as a foamy solid (3.7 g, 80%).

To a solution of compound 88 (3.6 g, 3.2 mmol) in dry CH₂Cl₂ (40 mL) was added BF₃.Et₂O (1.0 mL, 7.3 mmol), and the mixture was stirred at room temperature for 4 h, and TLC (petroleum ether-EtOAc, 1:1) indicated that the reaction was complete. The mixture was diluted with CH₂Cl₂, washed with satd aq NaHCO₃ and then satd aq NaCl. The organic layer was combined, dried, and concentrated. Purification by column chromatography (petroleum ether-EtOAc, 1:1) gave 89 as a white foamy solid (3.07 g, 95%): MALDITOF-MS: Calcd for C₆₁H₇₀O₁₃: 1010.48 [M]⁺. Found 1033.60 [M+Na]⁺

### Preparation of (90)

To a mixture of compound 87 (2.30 g, 3.10 mmol) and 89 (2.6 g, 2.6 mmol) in anhyd CH₂Cl₂ (50 mL) was added Me₃SiOTf (56 μL, 0.31 mmol) under N₂ atmosphere at 0° C. The mixture was stirred under r.t. for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 90 as a foamy solid (3.72 g, 90%): MALDITOF-MS: Calcd for C₉₅H₉₆O₂₂: 1588.64 [M]⁺. Found 1611.5 [M+Na]⁺.

### Preparation of (91)

The mixture of compound 90 (600 mg, 0.38 mmol) and NaBH₄ (357 mg, 9.4 mmol) in 2-propanol (16 mL) and CH₂Cl₂ (2 mL) was stirred at room temperature for about 7.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 91 as a white solid (393 mg, 65%): Calcd for C₉₅H₉₈O₂₂: 1590.65 [M]⁺. Found 1613.5 [M Na]⁺.

### Preparation of (92)

Compound 91 (210 mg, 0.13 mmol) was dissolved in anhyd CH₂Cl₂-MeOH (1:2, 24 mL), and then 1.0 M NaOMe in MeOH (0.25 mL) was added at 0° C. After stirring at room temperature for 5 h, TLC (n-BuOH-EtOH—H₂O, 2:0.5:05) indicated that the reaction was complete. The solution was neutralized with ion-exchange resin (H⁺), and then filtered and concentrated. The residue was purified by Bio-gel P₂ column to afford 92 as an amorphous solid (94 mg, 95%): Selected ¹H NMR (400 MHz, C₆D₅N); δ5.37 (d, 1 H, J 7.5 Hz, H-1), 5.20 (d, 1H, J 7.2 Hz, H-1. ¹³C NMR (100 MHz, CD₅N): δ 105.5, 103.1. MALDITOF-MS: Calcd for C₃₉H₆₆O₁₄: 758.45 [M]⁺. Found 781.31 [M Na]⁺.

### Example 21

### The Preparation of (25S)-26-O-β-D-galactopyranosyl-22-hydroxy-5β-furostane-3β,26-diol-3-O-β-D-glucopyranosyl-(1→4)-β-D-glucopyranoside (95)

The overall reaction scheme is as follows:

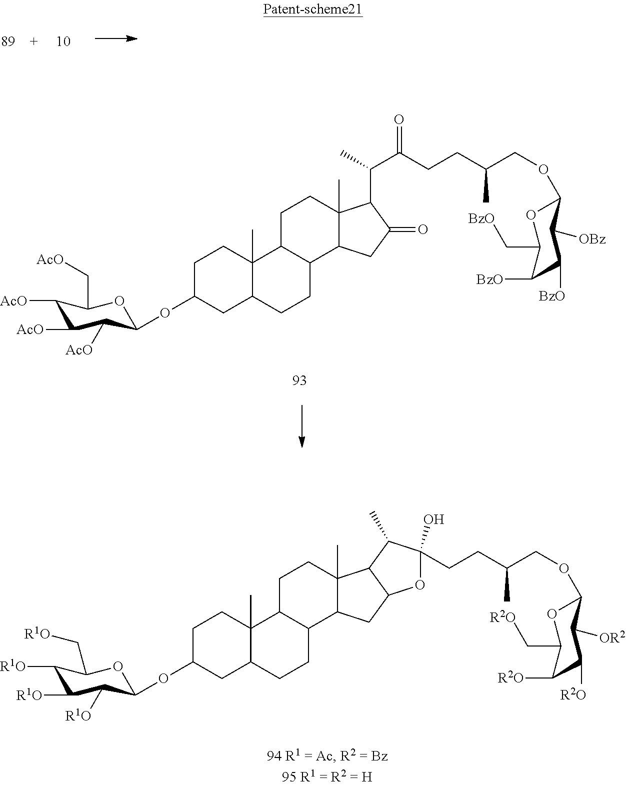

### Preparation of (93)

To a mixture of compound 10 (1.54 g, 3.12 mmol) and 89 (2.6 g, 2.6 mmol) in anhyd CH₂Cl₂ (30 mL) was added Me₃SiOTf (56 μL, 0.31 mmol) under N₂ atmosphere at 0° C. The mixture was stirred under room temperature for 15 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 89 as a foamy solid (2.96 g, 85%): MALDITOF-MS: Calcd for C₇₅H₈₈O₂₂: 1340.58 [M]⁺. Found 1363.70 [M+Na]⁺.

### Preparation of (94)

The mixture of compound 93 (114 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂, (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 94 as a white solid (74 mg, 65%). MALDITOF-MS: Calcd for C₇₃H₉₀O₂₂: 1342.59 [M]⁺. Found 1365.60 [M+Na]⁺.

### Preparation of (95)

Compound 94 (70 mg, 0.052 mmol) was dissolved in anhyd CH₂Cl₂-MeOH (1:2, 18 mL), and then 1.0 M NaOMe in MeOH (0.2 mL) was added at 0° C. After stirring at room temperature for 5.5 h, TLC (n-BuOH-EtOH—H₂O, 2:0.5:0.5) indicated that the reaction was complete. The solution was neutralized with ion-exchange resin (H^{α}), and then filtered and concentrated. The residue was purified by Bio-gel P₂ column to afford 95 as an amorphous solid (38 mg, 96%): Selected ¹H NMR (400 MHz, C₆D₅N): δ 5.30 (d, 1H, J 7.9 Hz, H-1), 5.23 (d, 1H, J 7.4 Hz, H-1). ¹³C NMR (100 MHz, CD₅N): δ 104.5, 102.9. MALDITOF-MS: Calcd for C₃₉H₆₆O₁₄: 758.45 [M]⁺. Found 781.31 [M+Na]⁺.

### Example 22

### The Preparation of (25S)-26-O-β-D-galactopyranosyl-22-hydroxy-5β-furostane-3β,26-diol-3-O-β-D-glucopyranosyl-(1→4)-β-D-galactopyranoside (98)

The overall reaction scheme is as follows:

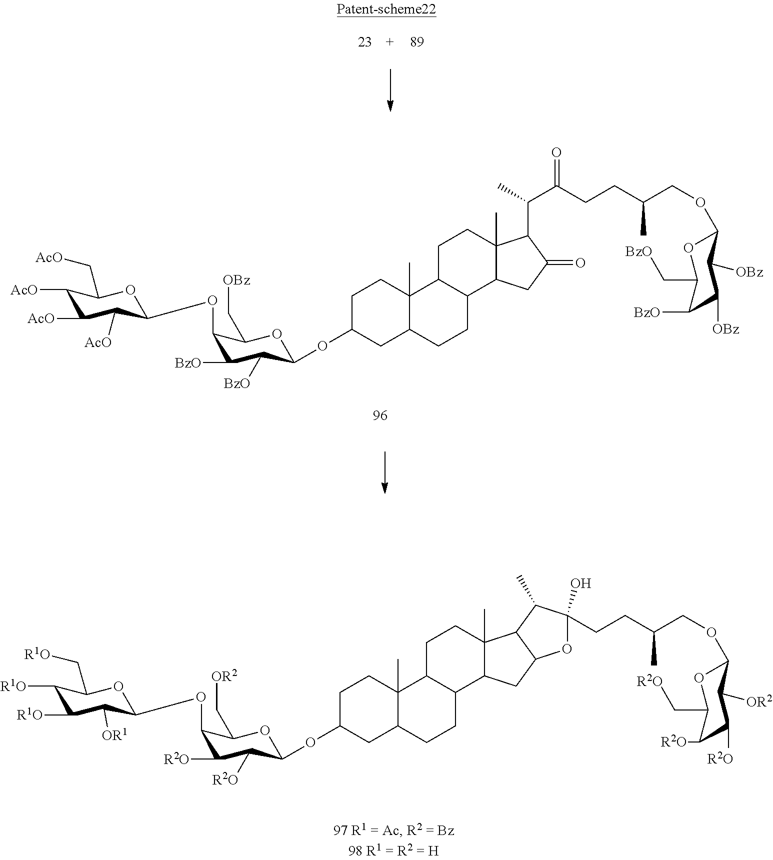

### Preparation of (96)

To a mixture of 23 (3.1 g, 3.52 mmol) and 89 (3.24 g, 3.2 mmol) in anhyd CH₂Cl₂ (55 mL) at −20° C. was added NIS (1.18 g, 5.28 mmol) and Me₃SiOTf (63 μL, 0.35 mmol) under N₂ atmosphere at −20° C. The mixture was stirred under these conditions for 30 min, at the end of which time TLC (petroleum ether-EtOAc, 3:2) indicated that all starting materials were consumed. The reaction mixture was neutralized with TEA, then concentrated. Column chromatography (petroleum ether-EtOAc, 2:1) of the residue gave 96 as a foamy solid (4.94 g, 85%): MALDITOF-MS: Calcd for C₁₀₂H₁₁₀O₃₆: 1814.71 [M]⁺. Found 1837.50 [M+Na]⁺.

### Preparation of (97)

The mixture of compound 96 (500 mg, 0.275 mmol) and NaBH₄ (313 mg, 8.26 mmol) in 2-propanol (24 mL) and CH₂Cl₂ (3 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 97 as a white solid (320 mg, 64%): MALDITOF-MS: Calcd for C₁₀₂H₁₁₂O₃₀: 1816.72 [M]⁺. Found 1839.50 [M+Na]⁺.

### Preparation of (98)

Compound 97 (200 mg, 0.11 mmol) was dissolved in anhyd CH₂Cl₂-MeOH (1:2, 21 mL), and then 1.0 M NaOMe in MeOH (0.22 mL) was added at 0° C. After stirring at room temperature for 4.5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 98 as an amorphous solid (95 mg, 94%): Selected ¹H NMR (400 MHz, C₆D₅N): δ5.35 (d, 1H, J 7.9 Hz, H-1), 5.30 (d, 1H, J 7.9 Hz, H-1), 5.20 (d, 1H, J 7.4 Hz, H-1). ¹³C NMR (100 MHz, CD₅N): δ 104.5, 103.3, 102.9. MALDITOF-MS: Calcd for C₄₅H₂₆O₁₉: 920.5 [M]⁺. Found 943.7 [M+Na]⁺.

### Example 23

### The Preparation of (25S)-26-O-β-D-galactopyranosyl-22-hydroxy-5β-furostane-3β,26-diol-3-O-β-D-glucopyranosyl-(1→2)-β-D-galactopyranoside (102)

The overall reaction scheme is as follows:

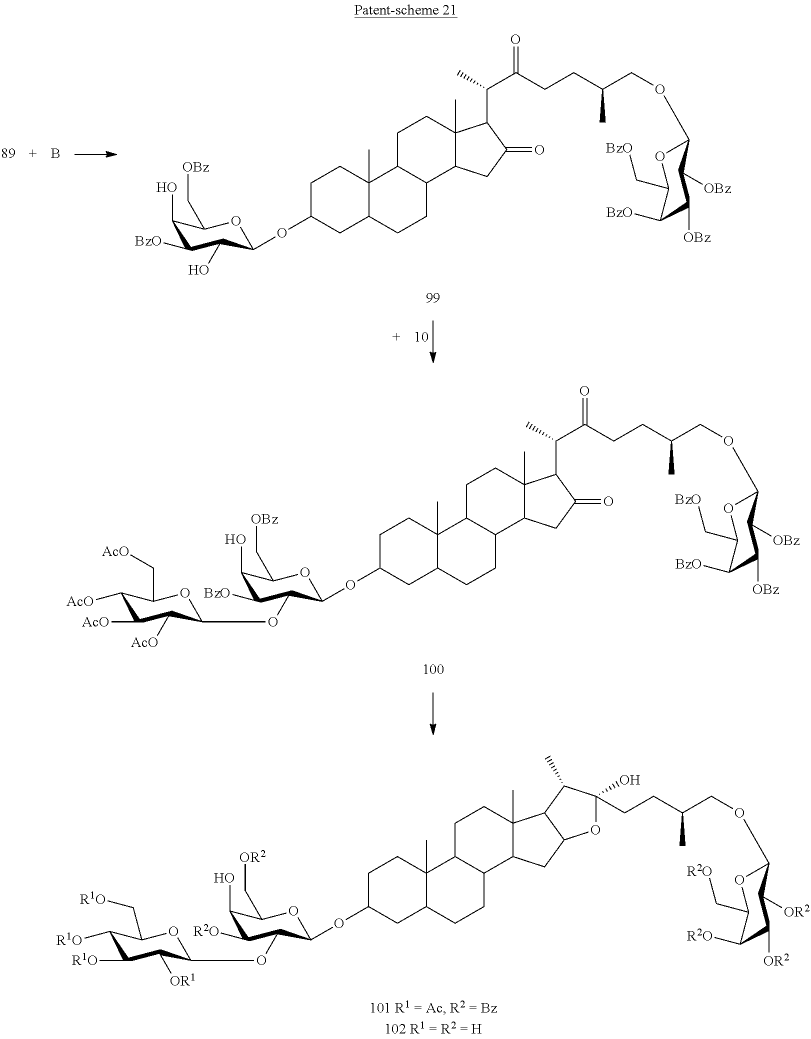

### Preparation of (99)

To a mixture of compound 8 (1.38 g, 3.08 mmol) and 89 (2.6 g, 2.6 mmol) in anhyd CH₂Cl₂ (50 mL) was added NIS (1.04 g, 4.6 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 99 as a foamy solid (2.94 g, 82%): MALDITOF-MS: Calcd for C₈₁H₈₈O₂₀: 1380.59 [M]⁺. Found 1403.70 [M+Na]⁺.

### Preparation of (100)

To a mixture of compound 10 (685 mg, 1.39 mmol) and 99 (1.6 g, 1.15 mmol) in anhyd CH₂Cl₂ (15 mL) was added Me₃SiOTf (26 μL, 0.14 mmol) under N₂ atmosphere at −42° C. The mixture was stirred under these conditions for 30 min, at which time TLC (petroleum ether-EtOAc, 1:1) indicated that all starting materials were consumed. The reaction mixture was neutralized with TEA, Then he mixture was concentrated with toluene and purified by column chromatography (petroleum ether-EtOAc, 3:1) to give 100 as a white foamy solid (1.5 g, 75%): MALDITOF-MS: Calcd for C₉₅H₁₀₆O₂₉: 1710.68 [M]⁺. Found 173335 [M Na]⁺.

### Preparation of (101)

The mixture of compound 100 (150 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 101 as a white solid (102 mg, 68%): MALDITOF-MS: Calcd for C₉₅H₁₀₆O₂₉: 1712.68 [M]⁺. Found 1735.75 [M+Na]⁺.

### Preparation of (102)

Compound 101 (100 mg, 0.057 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 102 as an amorphous solid (50 mg, 95%): Selected ¹H NMR (400 MHz, C₆D₅N): δ5.39 (d, 1H, J 7.9 Hz, H-1), 5.28 (d, 1H, J 7.9 Hz, H-1), 5.19 (d, 1H, J 7.4 Hz, H-1). ¹³C NMR (100 MHz, CD₅N): δ104.0, 103.1, 101.9. MALDITOF-MS: Calcd for C₄₅H₇₆O₁₉: 920.5 [M]⁺. Found 943.95 [M+Na]⁺.

### Example 24

### The Preparation of (25S)-26-O-β-D-galactopyranosyl-22-hydroxy-5β-furostane-3β,26-diol-3-O-β-D-glucopyranosyl-(1→4)-β-D-glucopyranoside (106)

The overall reaction scheme is as follows:

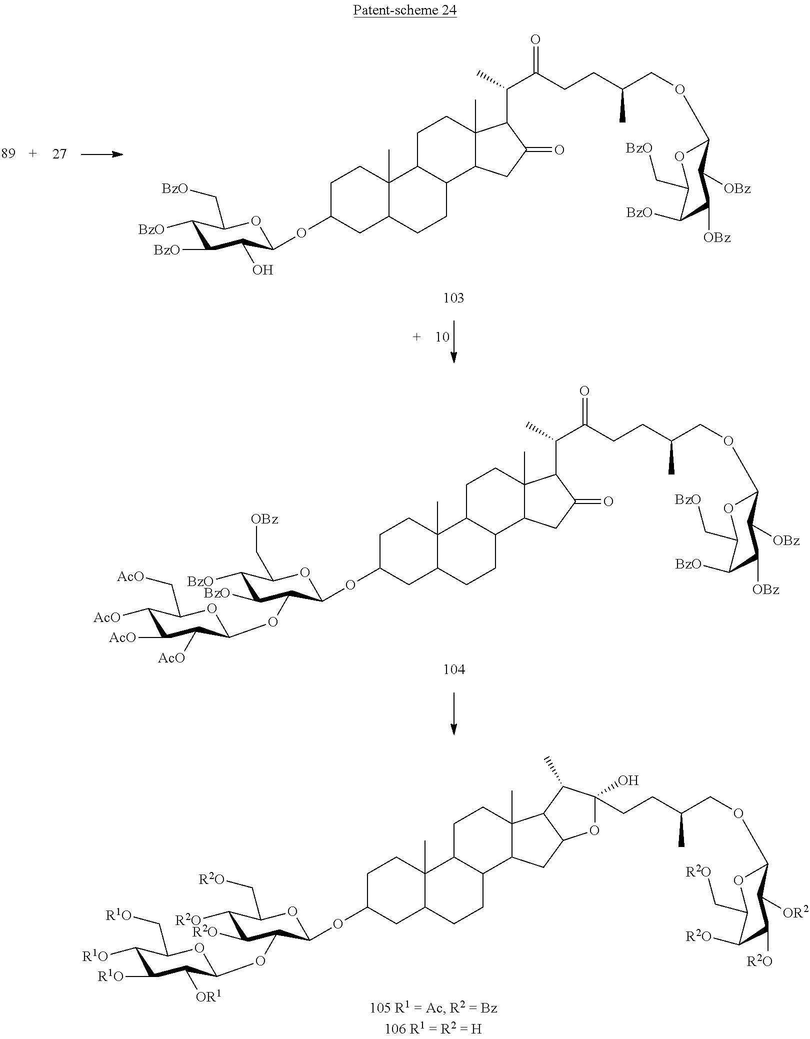

### Preparation of (103)

To a mixture of compound 27 (1.57 g, 2.86 mmol) and 89 (2.6 g, 2.6 mmol) in anhyd CH₂Cl₂ (35 mL) was added MS (643 mg, 2.86 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 103 as a foamy solid (3.09 g, 80%). MALDITOF-MS: Calcd for C₈₈H₉₂O₂₁: 1484.61 [M]⁺. Found 1507.80 [M+Na]⁺.

### Preparation of (104)

To a mixture of compound 10 (834 mg, 1.69 mmol) and 103 (2.1 g, 1.41 mmol) in anhyd CH₂Cl₂ (18 mL) was added Me₃SiOTf (31 μL, 0.17 mmol) under N₂ atmosphere at −20° C. The mixture was stirred under these conditions for 20 min, at which time TLC (petroleum ether-EtOAc, 1:1) indicated that all starting materials were consumed. The reaction mixture was neutralized with TEA, then concentrated to afford 104 as a white foamy solid (2.0 g, 78%). MALDITOF-MS: Calcd for C₁₀₂H₁₁₀O₃₀: 1814.71 [M]⁺. Found 1837.8 [M+Na]⁺.

### Preparation of (105)

The mixture of compound 104 (600 mg, 0.33 mmol) and NaBH₄ (312 mg, 8.25 mmol) in 2-propanol (24 mL) and CH₂Cl₂ (3 mL) was stirred at room temperature for about 6.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 105 as a white solid (313 mg, 67%). MALDITOF-MS: Calcd for C₁₀₂H₁₁₂O₃₀: 1816.71 [M]⁺. Found 1839.8 [M+Na]⁺.

### Preparation of (106)

Compound 105 (200 mg, 0.14 mmol) was dissolved in anhyd CH₂Cl₂-MeOH (1:2, 27 mL), and then 1.0 M NaOMe in MeOH (0.3 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₃ column to afford 106 as an amorphous solid (122 mg, 95%): Selected NMR (400 MHz, C₆D₅N): δ5.28 (d, 1H, J 7.9 Hz, H-1), 5.21 (d, 1H, J 7.9 Hz, H-1), 5.20 (d, 1H, J 7.4 Hz, H-1). ¹³C NMR (100 MHz, CD₅N): δ104.1, 103.0, 102.5. MALDITOF-MS: Calcd for C₄₅H₇₆O₁₉: 920.5 [M]⁺. Found 944.0 [M+Na]⁺.

### Example 25

### The Preparation of (25S)-26-O-β-D-galactopyranosyl-22-hydroxy-5β-furostane-3β,26-diol-3-O-β-D-glucopyranosyl-(1→4)-β-D-glucopyranoside (109)

The overall reaction scheme is as follows:

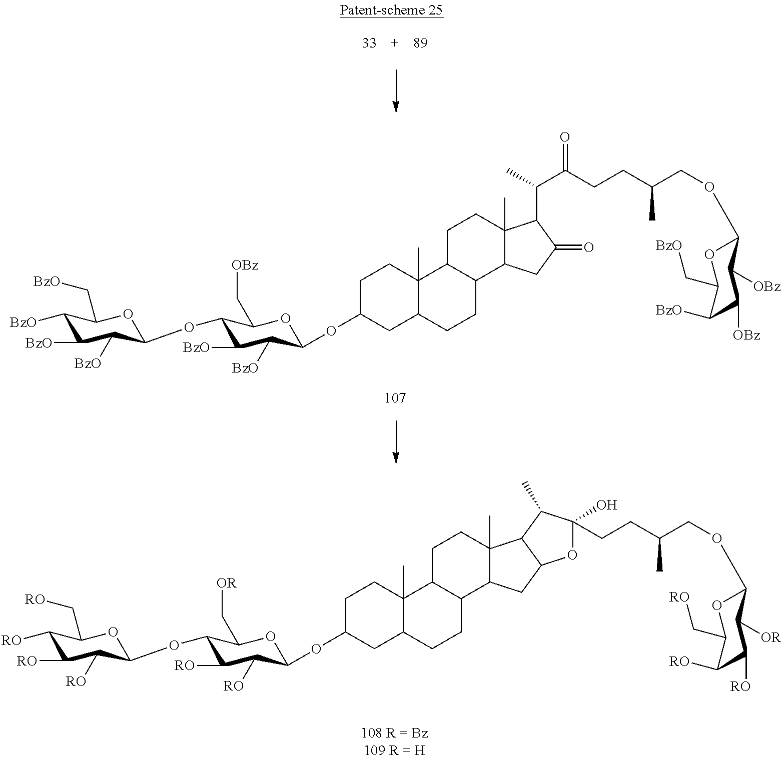

### Preparation of (107)

To a mixture of 33 (2.0 g, 1.8 mmol) and 89 (1.66 g, 1.64 mmol) in anhyd CH₂Cl₂ (30 mL) was added NIS (607 mg, 2.7 mmol) and Me₃SiOTf (33 μL, 0.18 mmol) under N₂ atmosphere at 0° C. The mixture was stirred under room temperature for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 107 as a foamy solid (2.88 g, 85%). MALDITOF-MS: Calcd for C₁₂₂H₁₁₈O₃₀: 2062.77 [M]⁺. Found 2085.90 [M+Na]⁺.

### Preparation of (108)

The mixture of compound 107 (175 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:0 to give compound 108 as a white solid (120 mg, 68%). MALDITOF-MS: Calcd for C₁₂₂H₁₂₀O₃₀: 2064.77 [M]⁺. Found 2087.90 [M Na]⁺.

### Preparation of (109)

Compound 108 (120 mg, 0.057 mmol) was dissolved in anhyd CH₂Cl₂-MeOH (1:2, 18 mL), and then 1.0 M NaOMe in MeOH (0.21 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 109 as an amorphous solid (50 mg, 95%): Selected ¹H NMR (400 MHz, C₆D₅N): δ5.38 (d, 1H, J 7.4 Hz, H-1), 5.32 (d, 1H, J 7.5 Hz, H-1), 5.11 (d, 1H, J 7.9 Hz, H-1). ¹³C NMR (100 MHz, CD₅N): δ103.5, 103.0, 102.1. MALDITOF-MS: Calcd for C₄₅H₇₆O₁₉: 920.50 [M]⁺. Found 943.90 [M+Na]⁺.

### Example 26

### The Preparation of (25S)-26-O-β-D-galactopyranosyl-22-hydroxy-5β-furostane-3β,26-diol-3-O-β-D-glucopyranosyl-(1→6)-β-D-glucopyranoside (112)

The overall reaction scheme is as follows:

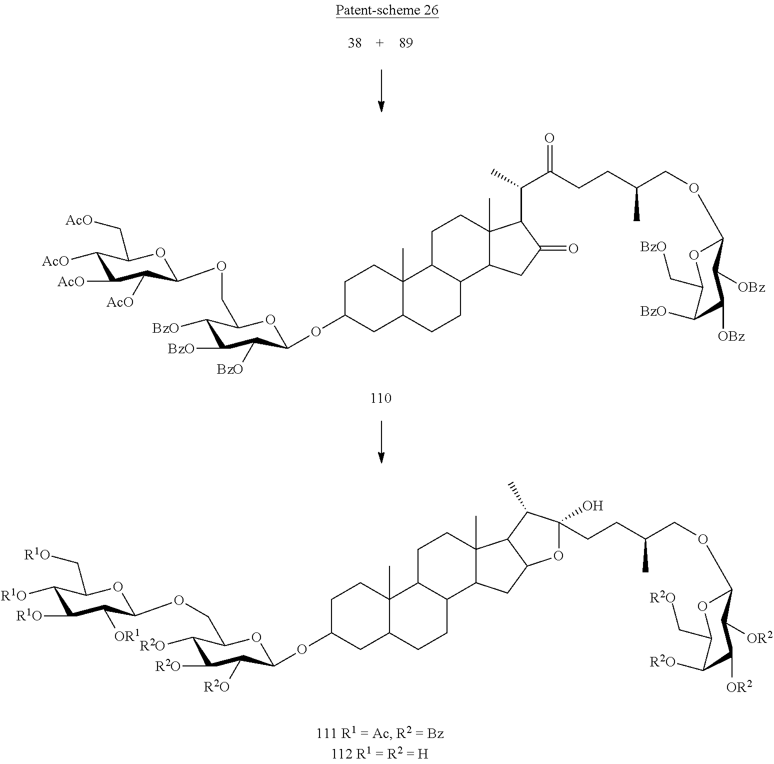

### Preparation of (110)

To a mixture of 38 (2.1 g, 2.4 mmol) and 89 (2.02 g, 2.0 mmol) in anhyd CH₂Cl₂ (35 mL) at −20° C. was added NIS (810 mg, 3.6 mmol) and Me₃SiOTf (43 μL, 0.24 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 110 as a foamy solid (2.98 g, 82%). MALDITOF-MS: Calcd for C₁₀₂H₁₁₀O₃₀: 1814.71 [M]⁺. Found 1837.8 [M+Na]⁺.

### Preparation of (111)

The mixture of compound 110 (154 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound III as a white solid (99 mg, 64%). MALDITOF-MS: Calcd for C₁₀₂H₁₁₂O₃₀: 1816.71 [M]⁺. Found 1839.8 [M+Na]⁺.

### Preparation of (112)

Compound 111 (80 mg, 0.056 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 111 as an amorphous solid (50 mg, 96%): Selected ¹H NMR (400 MHz, C₆D₅N): δ5.41 (d, 1H, J 7.1 Hz, H-1), 5.33 (d, 1H, J 7.5 Hz, H-1), 5.12 (d, 1H, J 7.2 Hz, H-1). ¹³C NMR (100 MHz, CD₅N): δ103.7, 103.0, 102.5. MALDITOF-MS: Calcd for C₄₅H₇₆O₁₉: 920.5 [M]⁺. Found 943.8 [M+Na]⁺.

### Example 27

### The Preparation of (25S)-26-β-D-galactopyranosyl-22-hydroxy-5β-furostane-3β,26-diol-3-O-α-L-rhamnopyranosyl-(1→2)-β-D-glucopyranoside (115)

The overall reaction scheme is as follows:

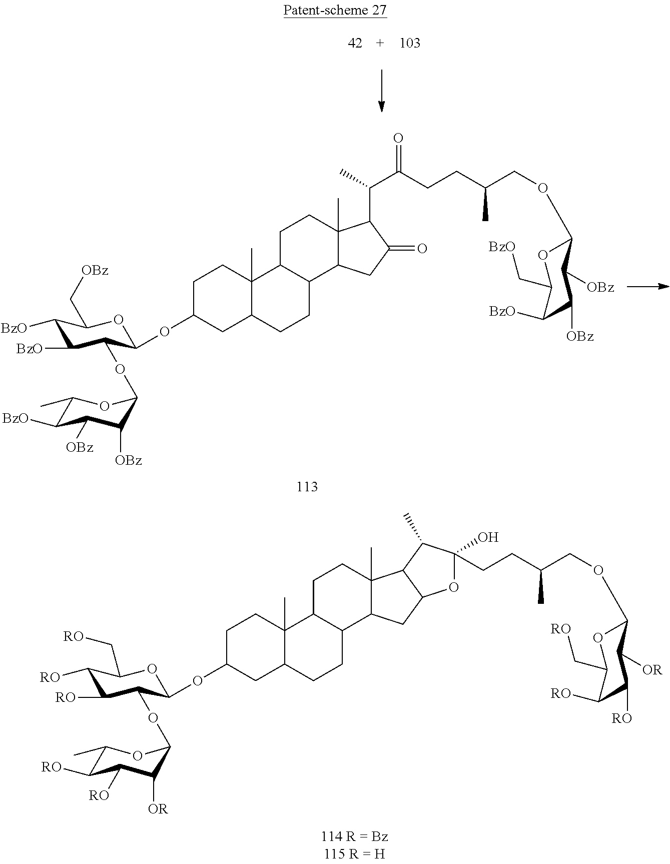

### Preparation of (113)

To a mixture of compound 42 (1.5 g, 2.4 mmol) and 103 (3.27 g, 2.2 mmol) in anhyd CH₂Cl₂ (50 mL) at −20° C. was added TMSOTf (44 μL, 0.24 mmol). The mixture was stirred at these conditions for 40 min, then neutralized with TEA, and concentrated. The residue was purified by column chromatography to give a foamy solid 113 (3.68 g, 86%). MALDITOF-MS: Calcd for C₁₁₅H₁₁₄O₂₈: 1942.75 [M]⁺. Found 1965.60 [M+Na]⁺.

### Preparation of (114)

The mixture of compound 113 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 114 as a white solid (115 mg, 70%). MALDITOF-MS: Calcd for C₁₁₅H₁₁₆O₂₈: 1944.75 [M]⁺. Found 1967.60 [M+Na]⁺.

### Preparation of (115)

Compound 114 (100 mg, 0.051 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 115 as an amorphous solid (44 mg, 95%): Selected ¹H NMR (400 MHz, C₆D₅N): δ5.53 (d, 1H, J 2.5 Hz, H-1), 5.30 (d, 1H, J 7.1 Hz, H-1), 5.20 (d, 1H, J 7.2 Hz, H-1). ¹³C NMR (100 MHz, CD₅N): δ105.5, 103.1, 102.2. MALDITOF-MS: Calcd for C₄₅H₇₆O₁₈: 904.50 [M]⁺. Found 947.80 [M+Na]⁺.

### Example 28

### The Preparation of (25S)-26-O-β-D-galactopyranosyl-22-hydroxy-5β-furostane-3β,26-diol-3-O-α-L-rhamnopyranosyl-(1→4)-β-D-glucopyranoside (118)

The overall reaction scheme is as follows:

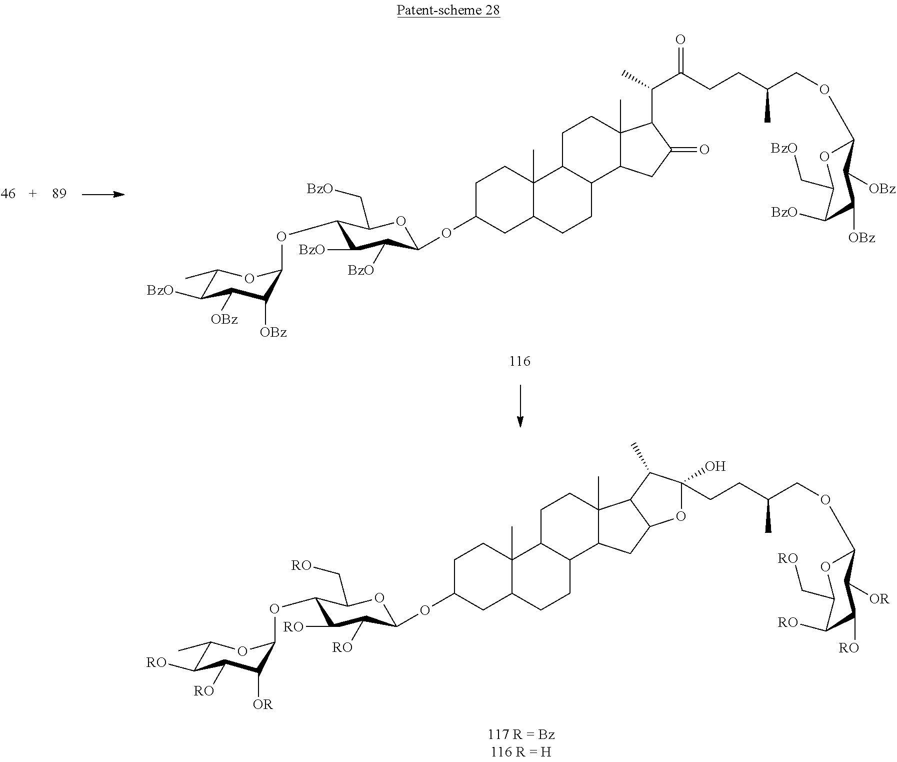

### Preparation of (116)

To a mixture of 46 (2.1 g, 2.11 mmol) and 89 (1.94 g, 1.92 mmol) in anhyd CH₂Cl₂ (45 mL) at −20° C. was added NIS (712 mg, 3.2 mmol) and Me₃SiOTf (38 μL, 0.211 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 116 as a foamy solid (3.17 g, 85%). MALDITOF-MS: Calcd for C₁₁₅H₁₁₄O₂₈: 1942.75 [M]⁺. Found 1965.60 [M+Na]⁺.

### Preparation of (117)

The mixture of compound 116 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 117 as a white solid (115 mg, 70%). MALDITOF-MS: Calcd for C₁₁₅H₁₁₆O₂₆: 1944.75 [M]⁺. Found 1967.60 [M+Na]⁺.

### Preparation of (118)

Compound 117 (100 mg, 0.051 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 118 as an amorphous solid (44 mg, 95%): Selected ¹H NMR (400 MHz, C₆D₅N): δ5.53 (d, 1H, J 2.6 Hz, H-1), 5.21 (d, 1H, J 7.5 Hz, H-1), 5.19 (d, 1H, J 7.4 Hz, H-1). ¹³C NMR (100 MHz, CD₅N): δ105.5, 103.7, 102.1. MALDITOF-MS: Calcd for C₄₅H₇₆O₃₈: 904.50 [M]⁺. Found 947.80 [M+Na]⁺.

### Example 29

### The Preparation of (25S)-26-O-β-D-galactopyranosyl-22-hydroxy-5β-furostane-3β,26-diol-3-O-α-L-rhamnopyranosyl-(1→6)-β-D-galactopyranoside (121)

The overall reaction scheme is as follows:

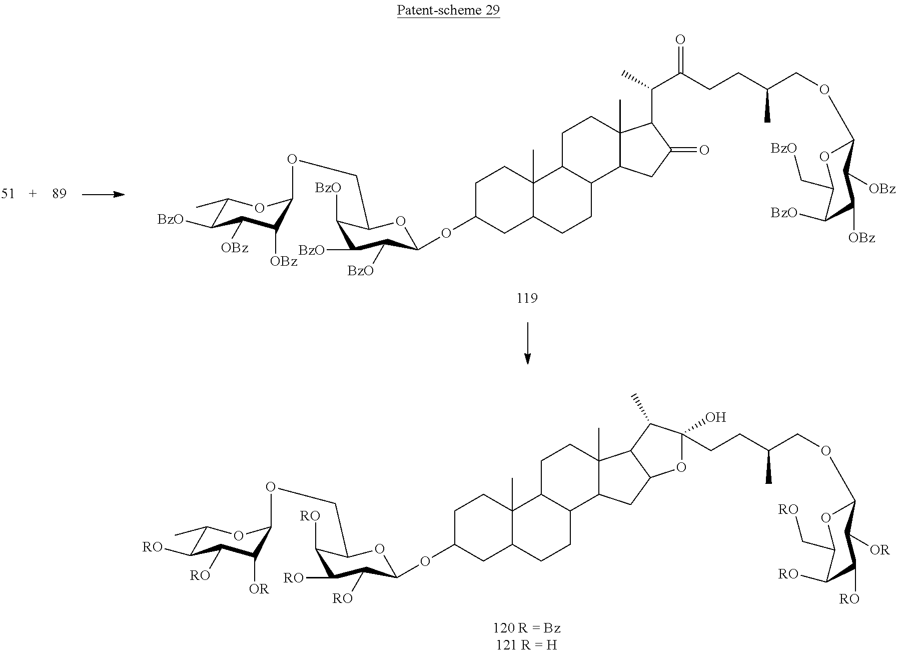

### Preparation of (119)

To a mixture of 51 (2.1 g, 2.11 mmol) and 89 (1.94 g, 1.92 mmol) in anhyd CH₂Cl₂ (40 mL) at −20° C. was added NIS (712 mg, 3.2 mmol) and Me₃SiOTf (38 μL, 0.211 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 119 as a foamy solid (3.17 g, 85%). MALDITOF-MS: Calcd for C₁₁₅H₁₁₄O₂₈: 1942.75 [M]⁺. Found 1965.60 [M+Na]⁺.

### Preparation of (120)

The mixture of compound 119 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 120 as a white solid (115 mg, 68%). MALDITOF-MS: Calcd for C₁₁₅H₁₁₆O₂₈: 1944.75 [M]⁺. Found 1967.60 [M+Na]⁺.

### Preparation of (121)

Compound 120 (100 mg, 0.057 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 121 as an amorphous solid (44 mg, 95%): Selected ¹H NMR (400 MHz, C₆D₅N): δ5.55 (d, 1H, J 2.7 Hz, H-1), 5.30 (d, 1H, J 7.5 Hz, H-1), 5.16 (d, 1H, J 7.2 Hz, H-1). ¹³C NMR (100 MHz, CD₅N): δ105.9, 103.4, 102.5. MALDITOF-MS: Calcd for C₄₅H₇₆O₁₈: 904.50 [M]⁺. Found 927.80 [M+Na]⁺.

### Example 30

### The Preparation of (25S)-26-O-β-D-galactopyranosyl-22-hydroxy-5β-furostane-3β,26-diol-3-O-[α-L-rhamnopyranosyl-(1→4)]-[α-L-rhamnopyranosyl-(1→2)]-β-D-glucopyranoside (125)

The overall reaction scheme is as follows:

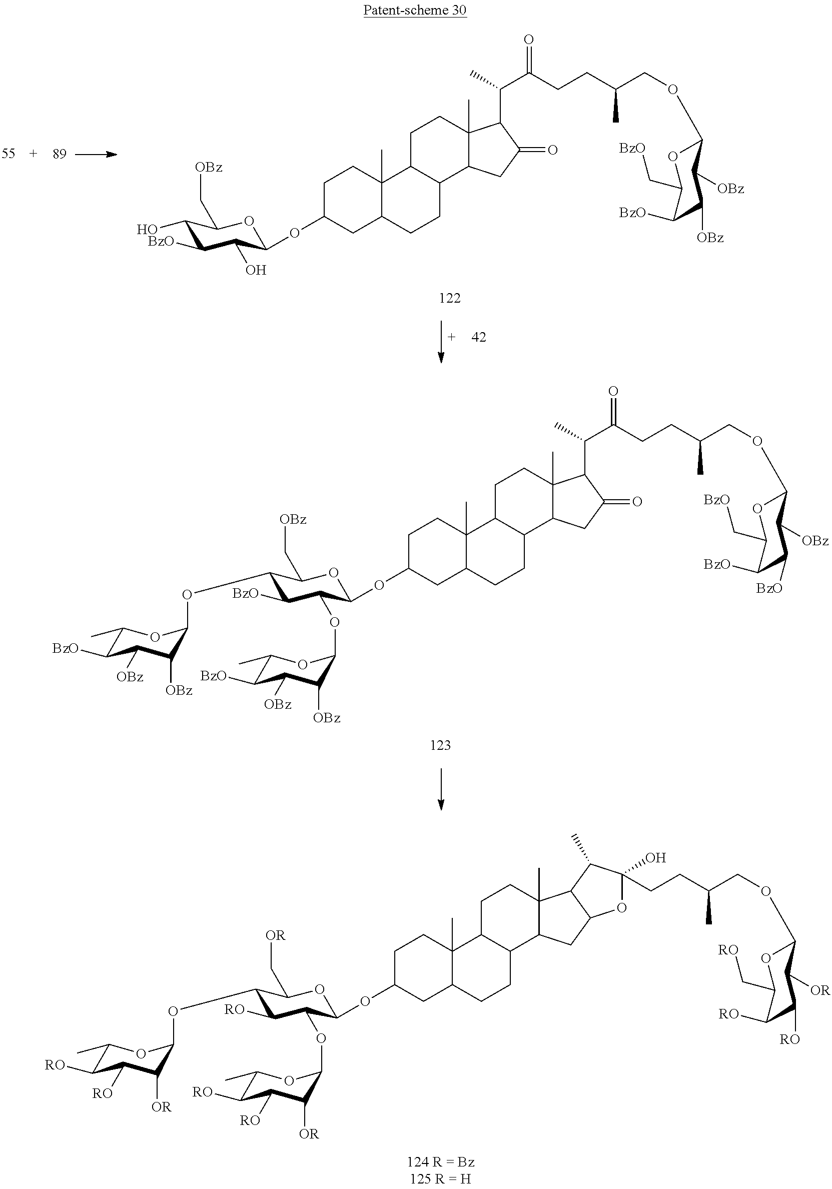

### Preparation of (122)

To a mixture of 55 (996 mg, 2.30 mmol) and 89 (1.94 g, 1.92 mmol) in anhyd CH₂Cl₂ (20 mL) at −40° C. was added NIS (517 mg, 2.30 mmol) and Me₃SiOTf (42 μL, 0.23 mmol) under N₂ atmosphere at −20° C. The mixture was stirred under these conditions for 40 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 122 as a foamy solid (2.12 g, 80%), MALDITOF-MS: Calcd for C₈₁H₈₈O₂₀: 1380.59 [M]⁺. Found 1403.70 [M Na]⁺.

### Preparation of (123)

To a mixture of 122 (2.0 g, 1.45 mmol) and 42 (2.16 g, 3.48 mmol) in anhyd CH₂Cl₂ (30 mL) at −20° C. was added Me₃SiOTf (62 μL, 0.34 mmol) under N₂ atmosphere at −20° C. The mixture was stirred under these conditions for 30 min, at the end of which time TLC (petroleum ether-EtOAc, 3:1) indicated that all starting materials were consumed. The reaction mixture was neutralized with TEA, then concentrated. Column chromatography (petroleum ether-EtOAc, 2:1) of the residue gave 123 as a foamy solid (2.83 g, 85%). MALDITOF-MS: Calcd for C₁₃₅H₁₃₂O₃₄: 2296.86 [M]⁺. Found 2319.90 [M+Na]⁺.

### Preparation of (124)

The mixture of compound 123 (195 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 124 as a white solid (121 mg, 68%). MALDITOF-MS: Calcd for C₁₃₅H₁₃₄O₃₄: 2298.86 [M]⁺. Found 2321.90 [M+Na]⁺.

### Preparation of (125)

Compound 124 (120 mg, 0.052 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:1) indicated that the reaction was complete. The solution was neutralized with ion-exchange resin (H⁺), and then filtered and concentrated. The residue was purified by Bio-gel P₂ column to afford 125 as an amorphous solid (53 mg, 97%): Selected ¹H NMR (400 MHz, C₆D₅N): δ5.53 (d, 1H, J 2.5 Hz, H-1), 5.50 (d, 1H, J 2.6 Hz, H-1), 5.22 (d, 1H, J 7.3 Hz, H-1), 5.17 (d, 1H, J 7.2 Hz, H-1). ¹³C NMR (100 MHz, CD₅N): δ 105.5, 104.8, 103.0, 102.5. MALDITOF-MS: Calcd for C₅₁H₈₆O₂₁: 1050.56 [M]⁺. Found 1073.80 [M+Na]⁺.

### Example 31

### The Preparation of (25S)-26-O-β-D-galactopyranosyl-22-hydroxy-5β-furostane-3β,26-diol-3-O-[β-D-glucopyranosyl-(1→4)]-[α-L-rhamnopyranosyl-(1→2)]-β-D-glucopyranoside (128)

The overall reaction scheme is as follows:

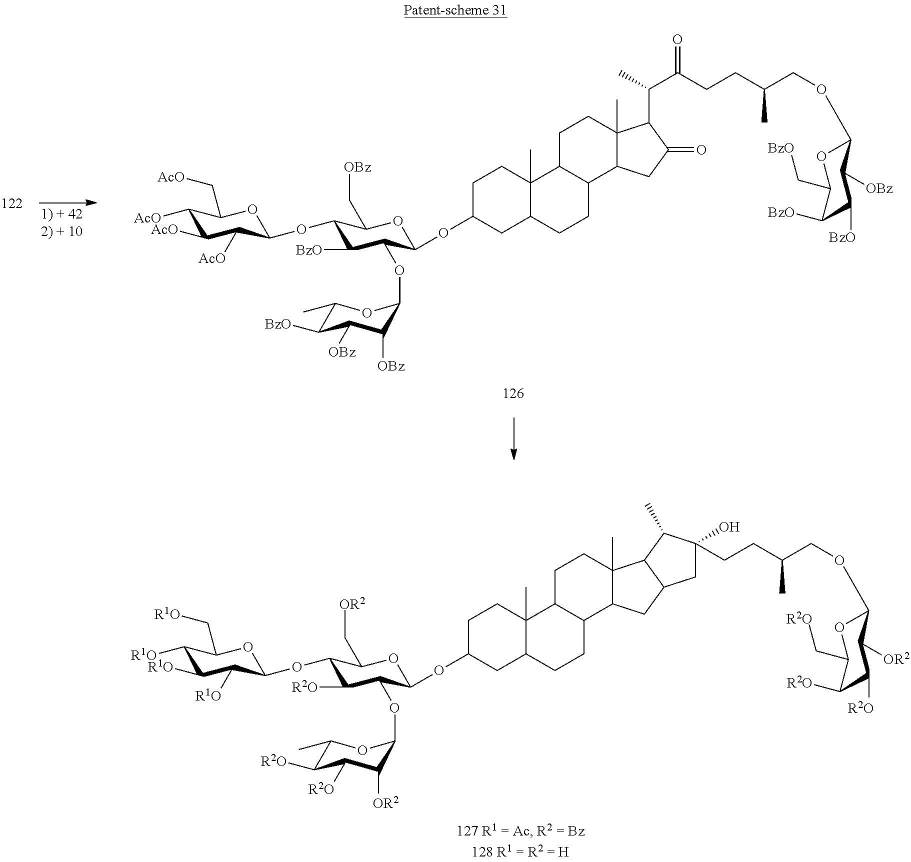

### Preparation of (126)

To a mixture of compound 122 (2.5 g, 1.81 mmol) and 42 (1.35 g, 2.17 mmol) in anhyd CH₂Cl₂ (50 mL) at −40° C. was added TMSOTf (40 μL, 0.22 mmol). The mixture was stirred at these conditions for 40 min, then the mixture was added 10 (1.07 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 126 as a foamy solid (3.10 g, 79%). MALDITOF-MS: Calcd for C₁₂₂H₁₂₈O₃₆: 2168.82 [M]⁺. Found 2192.0 [M+Na]⁺.

### Preparation of (127)

The mixture of compound 126 (185 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 127 as a white solid (118 mg, 64%). MALDITOF-MS: Calcd for C₁₂₂H₁₃₀O₃₆: 2170.82 [M]⁺. Found 2194.0 [M+Na]⁺.

### Preparation of (128)

Compound 127 (100 mg, 0.046 mmol) was dissolved in anhyd CH₂Cl₂-MeOH (1:2, 18 mL), and then 1.0 M NaOMe in MeOH (0.2 mL) was added at 0° C. After stirring at room temperature for 5H, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 128 as an amorphous solid (47 mg, 95%): Selected ¹H NMR (400 MHz, C₆D₅N): δ5.53 (d, 1H, J 2.5 Hz, H-1), 5.30 (d, 1H, J 6.9 Hz, H-1), 5.20 (d, 1H, J 7.5 Hz, H-1), 5.09 (d, 1H, J 7.2 Hz, H-1). ¹³C NMR (100 MHz, CD₅N): δ 105.5, 103.8, 103.0, 102.1. MALDITOF-MS: Calcd for C₅₁H₈₆O₂₃: 1066.56 [M]⁺. Found 1089.72 [M+Na]⁺.

### Example 32

### The Preparation of (25S)-26-O-β-D-galactopyranosyl-22-hydroxy-5β-furostane-3β,26-diol-3-O-[α-L-rhamnopyranosyl-(1→4)]-[β-D-glucopyranosyl-(1→2)]-β-D-glucopyranoside (131)

The overall reaction scheme is as follows:

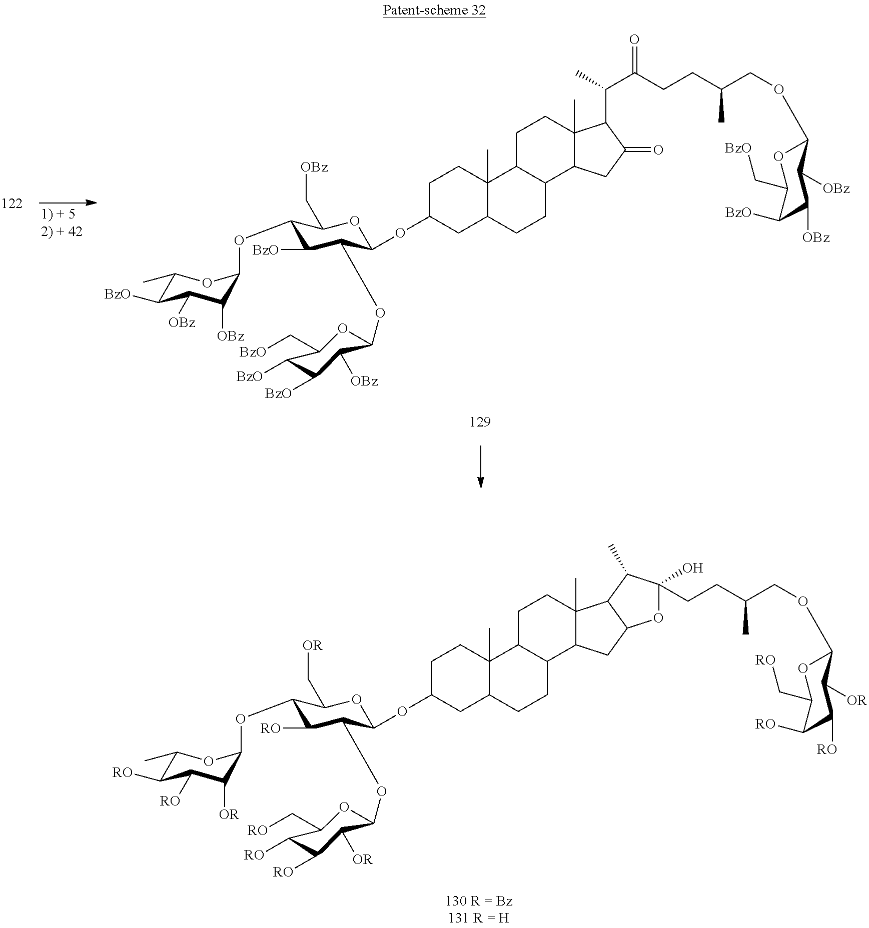

### Preparation of (129)

To a mixture of compound 122 (2.5 g, 1.81 mmol) and 5 (1.61 g, 2.17 mmol) in anhyd CH₂Cl₂ (50 mL) at −40° C. was added TMSOTf (40 μL, 0.22 mmol). The mixture was stirred at these conditions for 40 min, then the mixture was added 42 (1.35 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 129 as a foamy solid (3.46 g, 79%). MALDITOF-MS: Calcd for C₁₄₂H₁₃₆O₃₆: 2416.88 [M]⁺. Found 2439.75 [M+Na]⁺.

### Preparation of (130)

The mixture of compound 129 (206 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 130 as a white solid (123 mg, 60%). MALDITOF-MS: Calcd for C₁₄₂H₁₃₈O₃₆: 2418.88 [M]⁺. Found 2441.75 [M+Na]⁺.

### Preparation of (131)

Compound 130 (120 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 131 as an amorphous solid (51 mg, 95%): Selected ¹H NMR (400 MHz, C₆D₅N): δ5.56 (d, 1H, J 2.5 Hz, H-1), 5.22 (d, 1H, J 6.9 Hz, H-1), 5.19 (d, 1H, J 7.5 Hz, H-1), 5.10 (d, 1H, J 7.2 Hz, H-1). ¹³C NMR (100 MHz, CD₅N): δ 105.5, 103.1, 103.0, 102.6. MALDITOF-MS: Calcd for C₅₁H₈₆O₂₃: 1066.56 [M]⁺. Found 1089.72 [M+Na]⁺.

### Example 33

### The Preparation of (25S)-26-O-β-D-galactopyranosyl-22-hydroxy-5β-furostane-3β,26-diol-3-O-[β-D-glucopyranosyl-(1→4)]-[α-L-rhamnopyranosyl-(1→2)]-β-D-galactopyranoside (134)

The overall reaction scheme is as follows:

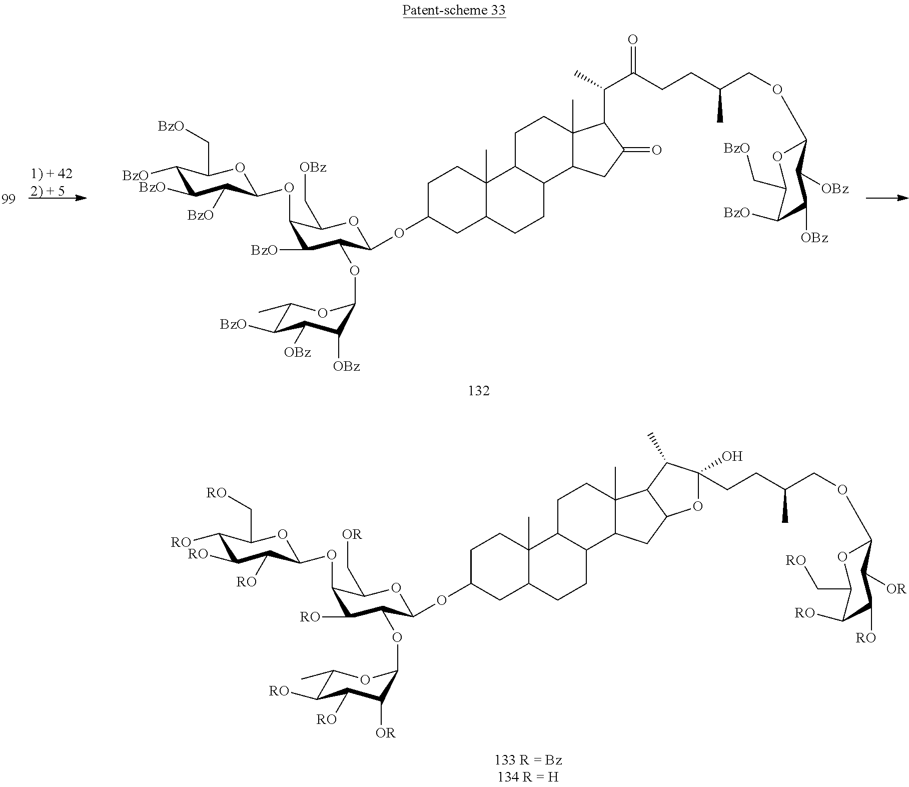

### Preparation of (132)

To a mixture of compound 99 (2.5 g, 1.81 mmol) and 42 (1.35 g, 2.17 mmol) in anhyd CH₂Cl₂ (50 mL) at −40° C. was added TMSOTf (40 μL, 0.22 mmol). The mixture was stirred at these conditions for 40 min, then the mixture was added 5 (1.61 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 132 as a foamy solid (3.46 g, 79%). MALDITOF-MS: Calcd for C₁₄₂H₁₃₆O₃₆: 2416.88 [M]⁺. Found 2439.75 [M+Na]⁺.

### Preparation of (133)

The mixture of compound 132 (206 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 133 as a white solid (123 mg, 60%). MALDITOF-MS: Calcd for C₁₄₂H₁₃₈O₃₆: 2418.88 [M]⁺. Found 2441.75 [M+Na]⁺.

### Preparation of (134)

Compound 133 (120 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio gel P₂ column to afford 134 as an amorphous solid (49 mg, 93%): Selected ¹H NMR (400 MHz, C₆D₅N): δ5.49 (d, 1H, J 2.5 Hz, H-1), 5.30 (d, 1H, J 6.9 Hz, H-1), 5.24 (d, 1H, J 17.5 Hz, H-1), 5.12 (d, 1H, J 7.2 Hz, H-1). ¹³C NMR (100 MHz, CD₅N): δ 105.5, 103.5, 103.1, 102.4. MALDITOF-MS: Calcd for C₅₁H₈₆O₂₃: 1066.56 [M]⁺. Found 1089.72 [M+Na]⁺.

### Example 34

### The Preparation of (25S)-26-O-β-D-galactopyranosyl-22-hydroxy-5β-furostane-3β,26-diol-3-O-[α-L-rhamnopyranosyl-(1→4)]-[β-D-glucopyranosyl-(1→2)]-β-D-galactopyranoside (137)

The overall reaction scheme is as follows:

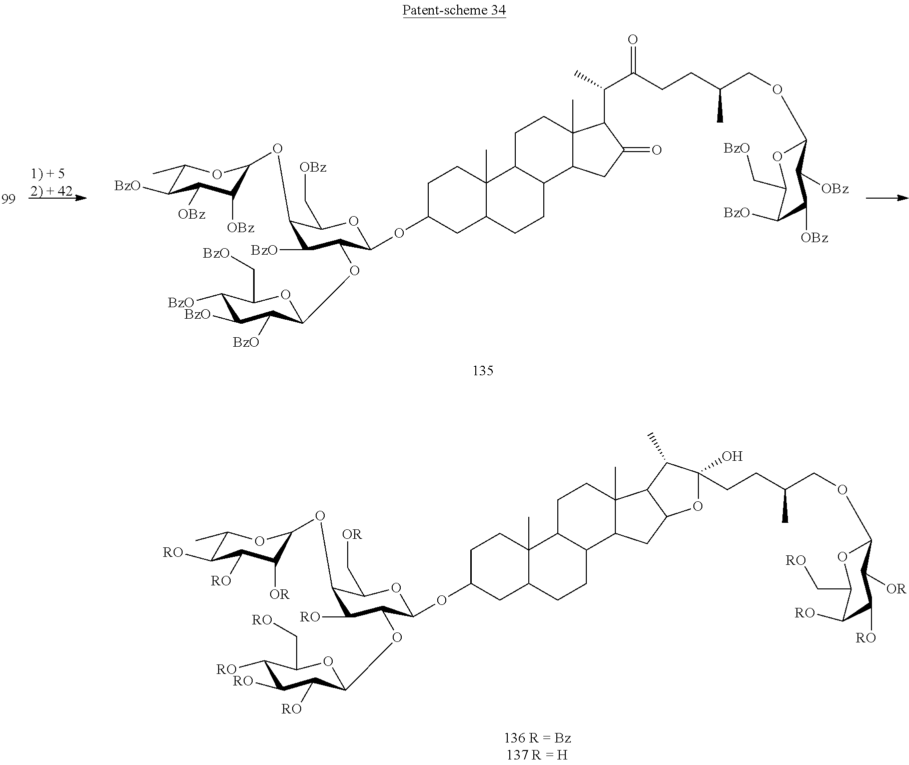

### Preparation of (135)

To a mixture of compound 99 (2.5 g, 1.81 mmol) and 5 (1.61 g, 2.17 mmol) in anhyd CH₂Cl₂ (50 mL) at −40° C. was added TMSOTf (40 μL, 0.22 mmol). The mixture was stirred at these conditions for 40 min, then the mixture was added 42 (1.35 g, 2.17 mmol) and TMSOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 135 as a foamy solid (3.46 g, 79%). MALDITOF-MS: Calcd for C₁₄₂H₁₃₆O₃₆: 2416.88 [M]⁺. Found 2439.75 [M+Na]⁺.

### Preparation of (136)

The mixture of compound 135 (206 mg, 0.085 mmol) and NaBH₄ (96 mg, 2.5 mmol) in 2-propanol (8 mL) and CH₂Cl₂ (1 mL) was stirred at room temperature for about 9 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 136 as a white solid (123 mg, 60%). MALDITOF-MS: Calcd for C₁₄₂H₁₃₈O₃₆: 2418.88 [M]⁺. Found 2441.75 [M+Na]⁺.

### Preparation of (137)

Compound 136 (120 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 137 as an amorphous solid (49 mg, 93%): Selected ¹H NMR (400 MHz, C₆D₅N): δ5.55 (d, 1H, J 2.5 Hz, H-1), 5.24 (d, 1H, J 6.9 Hz, H-1), 5.20 (d, 1H, J 7.5 Hz, H-1), 5.08 (d, 1H, J 7.2 Hz, H-1). ¹³C NMR (100 MHz, CD₅N): δ105.9, 103.2, 103.1, 102.5. MALDITOF-MS: Calcd for C₅₁H₈₆O₂₃: 1066.56 [M]⁺. Found 1089.72 [M+Na]⁺.

### Example 35

### The Preparation of (25S)-26-O-β-D-galactopyranosyl-22-hydroxy-5β-furostane-3β,26-diol-3-O-β-D-glucopyranosyl-(1→4)-β-D-glucopyranosyl-(1→4)-β-D-galactopyranoside (140)

The overall reaction scheme is as follows:

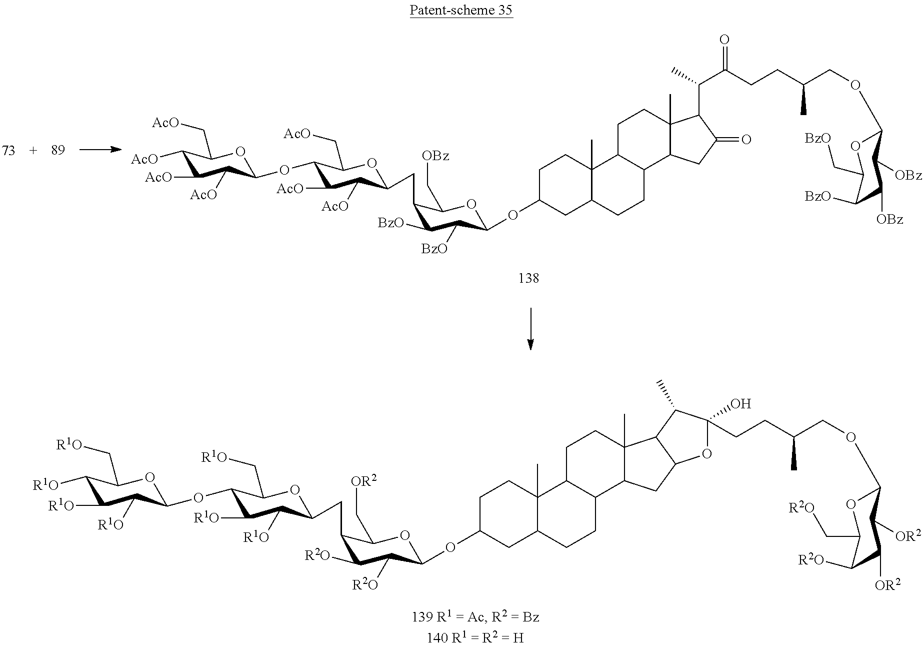

### Preparation of (138)

To a mixture of 73 (2.0 g, 1.7 mmol) and 89 (1.57 g, 1.55 mmol) in anhyd CH₂Cl₂ (50 mL) at −20° C. was added NIS (562 mg, 2.5 mmol) and Me₃SiOTf (31 μL, 0.17 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 138 as a foamy solid (2.71 g, 83%). MALDITOF-MS: Calcd for C₁₁₄H₁₂₆O₃₈: 2102.79 [M]⁺. Found 2125.90 [M+Na]⁺.

### Preparation of (139)

The mixture of compound 138 (179 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 139 as a white solid (116 mg, 67%). MALDITOF-MS: Calcd for C₁₁₄H₁₃₈O₃₈: 2104.79 [M]⁺. Found 2127.90 [M+Na]⁺.

### Preparation of (140)

Compound 139 (100 mg, 0.047 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 140 as an amorphous solid (49 mg, 96%): Selected ¹H NMR (400 MHz, C₆D₅N): δ5.35 (d, 1H, J 7.5 Hz, H-1), 5.30 (d, 1H, J 6.9 Hz, H-1), 5.27 (d, 1H, J 7.5 Hz, H-1), 5.05 (d, 1H, J 7.2 Hz, H-1). ¹³C NMR (100 MHz, CD₅N): δ 103.5, 103.1, 103.0, 102.5. MALDITOF-MS: Calcd for C₅₁H₈₆O₂₄: 1082.55 [M]⁺. Found 1105.70 [M+Na]⁺.

### Example 36

### The Preparation of (258)-26-O-β-D-galactopyranosyl-22-hydroxy-5β-furostane-3β,26-diol-3-O-β-D-glucopyranosyl-(1→4)-β-D-glucopyranosyl-(1→2)-β-D-galactopyranoside (143)

The overall reaction scheme is as follows:

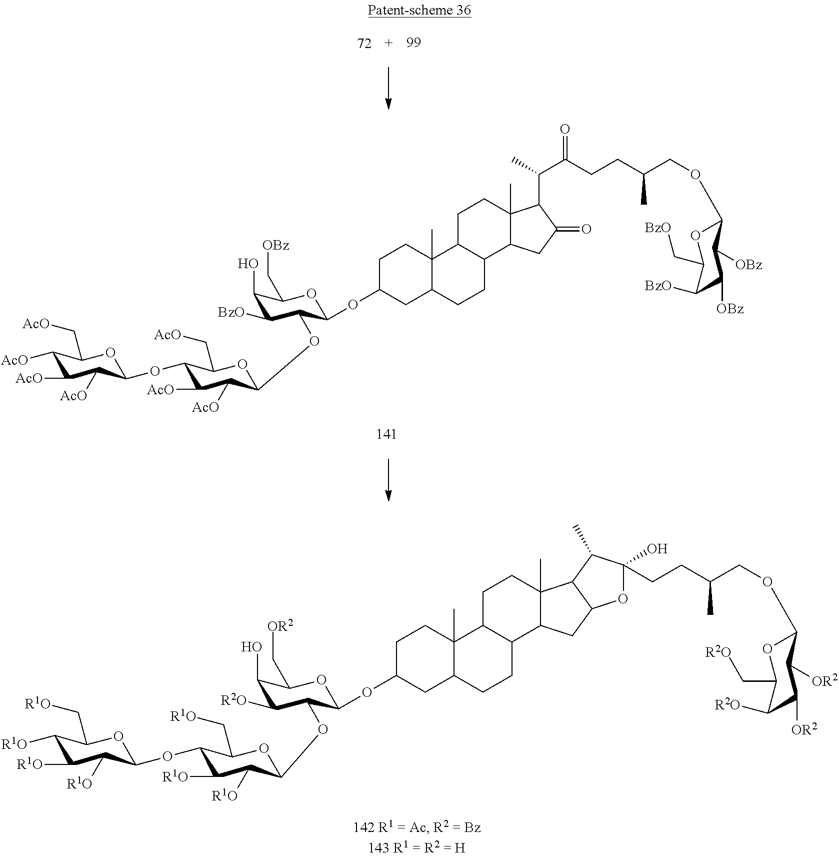

### Preparation of (141)

To a mixture of compound 72 (2.5 g, 3.20 mmol) and 99 (4.02 g, 2.91 mmol) in anhyd CH₂Cl₂ (50 mL) at −40° C. was added TMSOTf (58 μL, 0.32 mmol). The mixture was stirred at these conditions for 1 h, then neutralized with TEA, and concentrated. The residue was purified by column chromatography to give a foamy solid 141 (4.54 g, 78%). MALDITOF-MS: Calcd for C₁₀₇H₁₂₂O₃₇: 1998.77 [M]⁺. Found 2021.90 [M+Na]⁺.

### Preparation of (142)

The mixture of compound 141 (170 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 142 as a white solid (116 mg, 68%). MALDITOF-MS: Calcd for C₁₀₇H₁₂₄O₃₇: 2000.77 [M]⁺. Found 2023.90 [M+Na]⁺.

### Preparation of (143)

Compound 142 (100 mg, 0.05 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 143 as an amorphous solid (51 mg, 94%): Selected NMR (400 MHz, C₆D₅N): δ5.37 (d, 1H, J 6.9 Hz, H-1), 5.28 (d, 1H, J 7.6 Hz, H-1), 5.19 (d, 1H, J 7.5 Hz, H-1), 5.04 (d, 1H, J 7.2 Hz, H-1). ¹³C NMR (100 MHz, CD₅N): δ 103.1, 102.8, 102.5, 102.1. MALDITOF-MS: Calcd for C₅₁H₈₆O₂₄: 1082.55 [M]⁺. Found 1105.70 [M t Na]⁺.

### Example 37

### The Preparation of (25S)-26-O-β-D-galactopyranosyl-22-hydroxy-5β-furostane-3β,26-diol-3-O-β-D-glucopyranosyl-(1→4)-β-D-glucopyranosyl-(1→4)-β-D-glucopyranoside (146)

The overall reaction scheme is as follows:

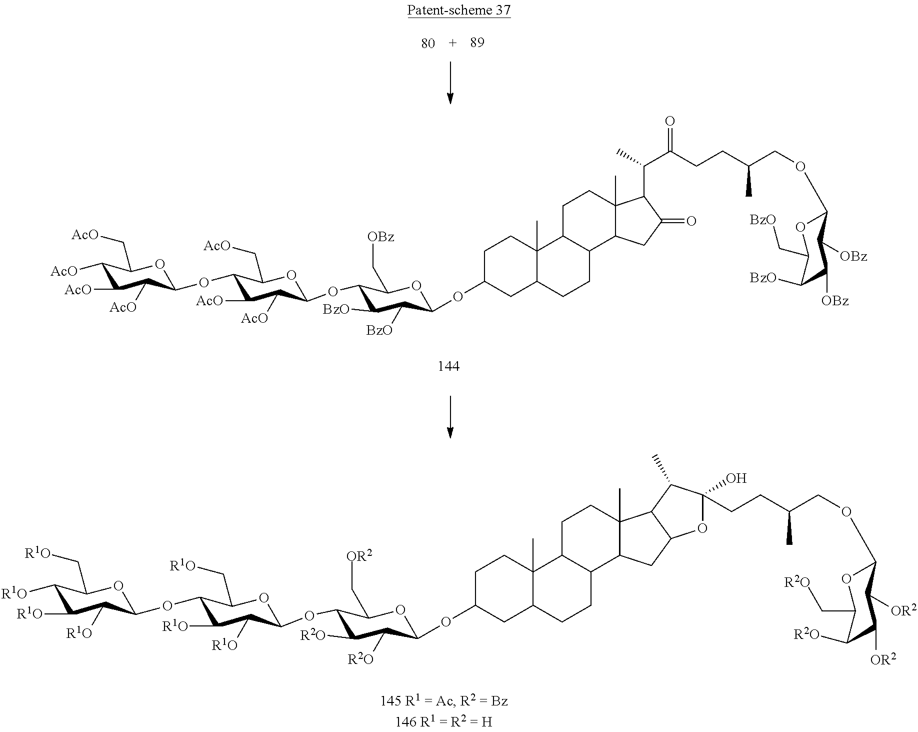

### Preparation of (144)

To a mixture of 80 (2.5 g, 2.16 mmol) and 89 (1.98 g, 1.96 mmol) in anhyd CH₂Cl₂ (50 mL) at −20° C. was added NIS (729 mg, 3.24 mmol) and Me₃SiOTf (40 μL, 0.22 mmol) under N₂ atmosphere at 0° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 1:1) of the residue gave 144 as a foamy solid (3.51 g, 85%). MALDITOF-MS: Calcd for C₁₁₄H₁₂₆O₃₈: 2102.79 [M]⁺. Found 2125.90 [M+Na]⁺.

### Preparation of (145)

The mixture of compound 144 (179 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 145 as a white solid (116 mg, 67%). MALDITOF-MS: Calcd for C₁₁₄H₁₂₈O₃₈: 2104.79 [M]⁺. Found 2127.90 [M+Na]⁺.

### Preparation of (146)

Compound 145 (100 mg, 0.047 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 146 as an amorphous solid (49 mg, 96%): Selected ¹H NMR (400 MHz, C₆D₅N): δ5.29 (d, 1H, J 7.1 Hz, H-1), 5.23 (d, 1H, J 6.9 Hz, H-1), 5.20 (d, 1H, J 7.5 Hz, H-1), 5.04 (d, 1H, J 7.2 Hz, H-1). ¹³C NMR (100 MHz, CD₅N): δ 103.6, 103.4, 102.8, 101.6. MALDITOF-MS: Calcd for C₅₁H₈₆O₂₄: 1082.55 [M]⁺. Found 1105.70 [M+Na]⁺.

### Example 38

### The Preparation of (25S)-26-O-β-D-galactopyranosyl-22-hydroxy-5β-furostane-3β,26-diol-3-O-β-D-glucopyranosyl-(1→4)-β-D-glucopyranosyl-(1→2)-β-D-glucopyranoside (149)

The overall reaction scheme is as follows;

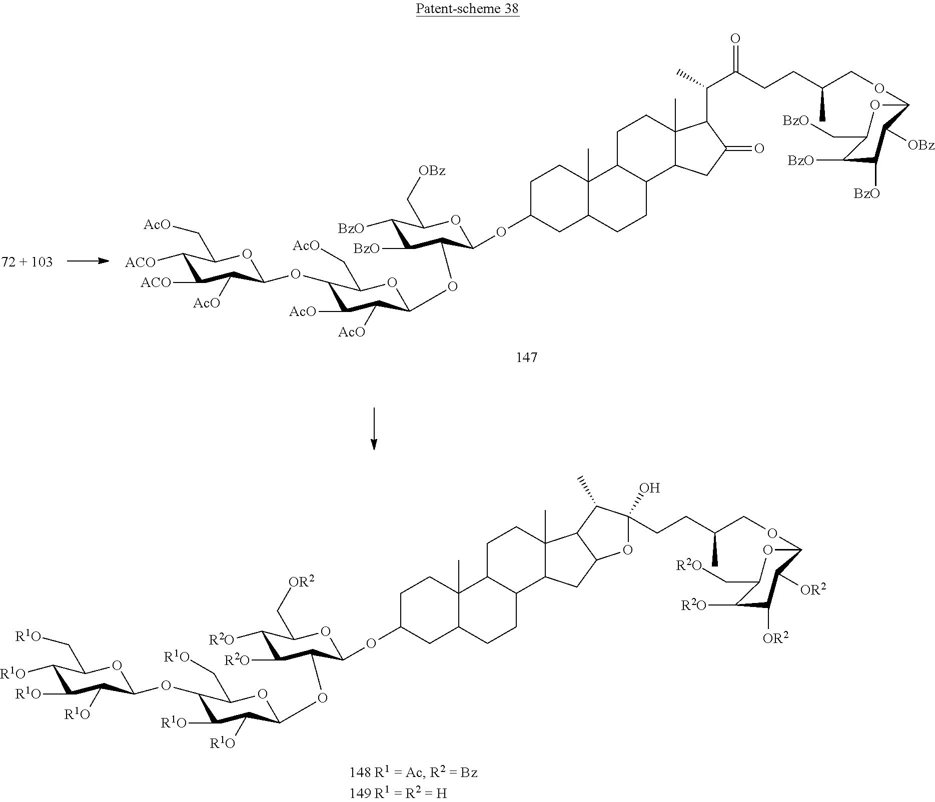

### Preparation of (147)

To a mixture of compound 103 (5.8 g, 3.9 mmol) and 72 (3.6 g, 4.6 mmol) in anhyd CH₂Cl₂ (55 mL) at −20° C. was added TMSOTf (83 μL, 0.46 mmol). The mixture was stirred at these conditions for 30 min, then neutralized with TEA, and concentrated. The residue was purified by column chromatography to give a foamy solid 147 (6.97 g, 85%), MALDITOF-MS: Calcd for C₁₁₄H₁₂₆O₃₈: 2102.79 [M]⁺. Found 2125.90 [M+Na]⁺.

### Preparation of (148)

The mixture of compound 147 (179 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂C17 (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 148 as a white solid (116 mg, 67%). MALDITOF-MS: Calcd for C₁₁₄H₁₂₈O₃₈: 2104.79 [M]⁺. Found 2127.90 [M+Na]⁺.

### Preparation of (149)

Compound 148 (100 mg, 0.047 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 149 as an amorphous solid (50 mg, 96%): Selected ¹H NMR (400 MHz, C₆D₅N): δ5.27 (d, 1H, J 7.5 Hz, H-1), 5.30 (d, 1H, J 7.9 Hz, H-1), 5.22 (d, 1H, J 7.5 Hz, H-1), 5.05 (d, 1H, J 7.3 Hz, H-1). ¹³C NMR (100 MHz, CD₅N): δ 103.5, 103.1, 102.3, 101.6. MALDITOF-MS: Calcd for C₅₁H₈₆O₂₄: 1082.55 [M]⁺. Found 1105.70 [M+Na]⁺.

### Example 39

### The Preparation of (25S)-26-O-α-L-rhamnopyranosyl-22-hydroxy-5β-furostane-3β,26-diol-3-O-β-D-galactopyranoside (154)

The overall reaction scheme is as follows:

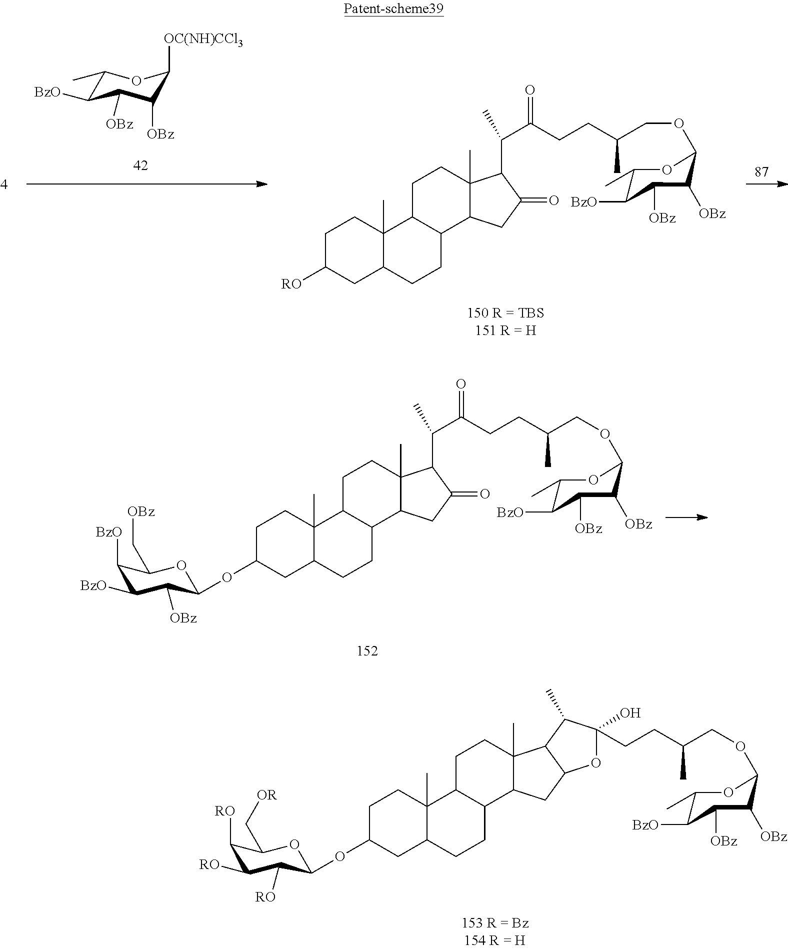

### Preparation of (151)

To a mixture of compounds 4 (2.25 g, 4.1 mmol) and 42 (2.98 g, 4.8 mmol, commercially available) in anhyd CH₂Cl₂ (36 mL) was added Me₃SiOTf (86 μL, 0.47 mmol) under N₂ atmosphere at −20° C. The mixture was stirred under these conditions for 40 min, at the end of which time TLC (petroleum ether-EtOAc, 4:1) indicated that all starting materials were consumed. The reaction mixture was neutralized with triethylamine (TEA), then concentrated. Column chromatography (petroleum ether-EtOAc, 6:1) of the residue gave 150 as a foamy solid (3.29 g, 80%).

To a solution of compound 150 (3.2 g, 3.2 mmol) in dry CH₂Cl₂ (40 mL) was added BF₃.Et₂O (1.0 mL, 7.3 mmol), and the mixture was stirred at room temperature for 4 h, and TLC (petroleum ether-EtOAc, 1:1) indicated that the reaction was complete. The mixture was diluted with CH₂Cl₂, washed with satd aq NaHCO₃ and then said aq NaCl. The organic layer was combined, dried, and concentrated. Purification by column chromatography (petroleum ether-EtOAc, 3:1) gave 151 as a white foamy solid (2.71 g, 95%): MALDITOF-MS: Calcd for C₃₄H₆₆O₁₁: 890.46 [M]⁺. Found 913.60 [M+Na]⁺.

### Preparation of (152)

To a mixture of compound 87 (2.30 g, 3.10 mmol) and 151 (2.32 g, 2.6 mmol) in anhyd CH₂Cl₂ (50 mL) was added Me₃SiOTf (56 μL, 0.31 mmol) under N₂ atmosphere at 0° C. The mixture was stirred under r.t. for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 152 as a foamy solid (3.44 g, 90%): MALDITOF-MS: Calcd for C₈₈H₉₂O₂₀: 1468.62 [M]⁺. Found 1491.75 [M+Na]⁺.

### Preparation of (153)

The mixture of compound 152 (558 mg, 0.38 mmol) and NaBH₄ (357 mg, 9.4 mmol) in 2-propanol (16 mL) and CH₂Cl₂ (2 mL) was stirred at room temperature for about 7.5 h. The reaction mixture was then extracted with CH₂Cl₂ (100 mL×2), the combined organic layer was washed with water (100 in L×3) and dried over anhydrous Na₂SO₄, and the solvent was removed under vacuum to give an colorless oil, which was purified by column chromatography (petroleum ether/ethyl acetate, 1:1) to give compound 153 as a white solid (363 mg, 65%): MALDITOF-MS: Calcd for C₈₈H₉₄O₂₀: 1470.62 [M]⁺. Found 1493.75 [M+Na]⁺.

### Preparation of (154)

Compound 153 (190 mg, 0.13 mmol) was dissolved in anhyd CH₂Cl₂-MeOH (1:2, 24 mL), and then 1.0 M NaOMe in MeOH (0.25 mL) was added at 0° C. After stirring at room temperature for 5 h, TLC (n-BuOH-EtOH—H₂O, 2:0.5:0.5) indicated that the reaction was complete. The solution was neutralized with ion-exchange resin an, and then filtered and concentrated. The residue was purified by Bio-gel P₂ column to afford 154 as an amorphous solid (92 mg, 95%): Selected ¹H NMR (400 MHz, C₆D₅N): δ5.53 (d, 1H, J 2.5 Hz, H-1), 5.30 (d, 1H, J 6.9 Hz, H-1). ¹³C NMR (100 MHz, CD₅N): δ105.5, 103.1. MALDITOF-MS: Calcd for C₃₉H₆₆O₁₃: 742.45 [M]⁺. Found 765.50 [M+Na]⁺.

### Example 40

### The Preparation of (25S)-26-O-α-L-rhamnopyranosyl-22-hydroxy-5β-furostane-3β,26-diol-3-O-β-D-glucopyranosyl-(1→2)-β-D-galactopyranoside (158)

The overall reaction scheme is as follows:

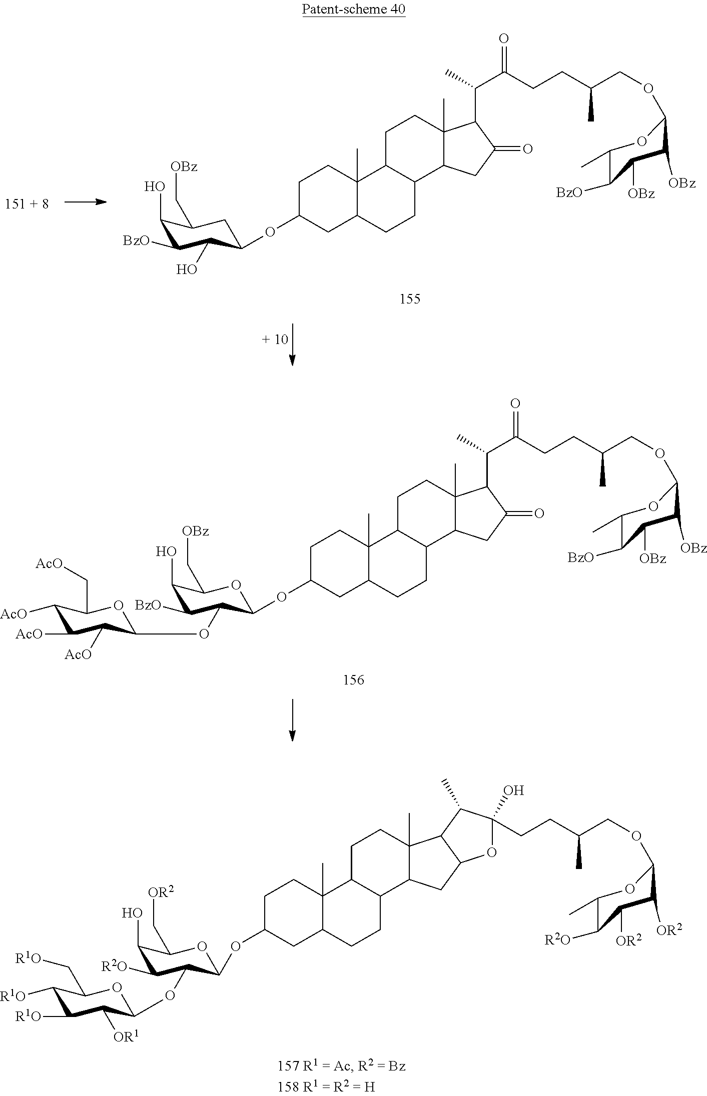

### Preparation of (155)

To a mixture of compound 8 (1.38 g, 3.08 mmol) and 151 (2.3 g, 2.6 mmol) in anhyd CH₂Cl₂ (50 mL) was added NIS (692 mg, 3.08 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 155 as a foamy solid (2.69 g, 82%): MALDITOF-MS: Calcd for C₇₄H₈₄O₁₈: 1260.57 [M]⁺. Found 1283.70 [M+Na]⁺.

### Preparation of (156)

To a mixture of compound 10 (685 mg, 1.39 mmol) and 155 (1.45 g, 1.15 mmol) in anhyd CH₂Cl₂ (15 mL) was added Me₃SiOTf (26 μL, 0.14 mmol) under N₂ atmosphere at −42° C. The mixture was stirred under these conditions for 30 min, at which time TLC (petroleum ether-EtOAc, 1:1) indicated that all starting materials were consumed. The reaction mixture was neutralized with TEA, Then he mixture was concentrated with toluene and purified by column chromatography (petroleum ether-EtOAc, 3:1) to give 156 as a white foamy solid (1.37 g, 75%): MALDITOF-MS: Calcd for C₈₈H₁₀₂O₂₇: 1590.66 [M]⁺. Found 1613.75 [M+Na]⁺.

### Preparation of (157)

The mixture of compound 156 (135 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 157 as a white solid (88 mg, 65%): MALDITOF-MS: Calcd for C₈₈H₁₀₄O₂₇: 1592.66 [M]⁺. Found 1615.75 [M+Na]⁺.

### Preparation of (158)

Compound 157 (80 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 158 as an amorphous solid (43 mg, 95%): Selected ¹H NMR (400 MHz, C₆D₅N): δ5.53 (d, 1H, J 2.5 Hz, H-1), 5.25 (d, 1H, J 7.5 Hz, H-1), 5.09 (d, 1H, J 7.2 Hz, H-1). ¹³C NMR (100 MHz, CD₅N): δ105.5, 103.0, 102.1. MALDITOF-MS: Calcd for C₄₅H₇₆O₁₈: 904.50 [M]⁺. Found 927.75 [M+Na]^{α}.

### Example 41

### The Preparation of (25S)-26-O-α-L-rhamnopyranosyl-22-hydroxy-5β-furostane-3β,26-diol-3-O-[α-L-rhamnopyranosyl-(1→4)]-[α-L-rhamnopyranosyl-(1→2)]-β-D-glucopyranoside (162)

The overall reaction scheme is as follows:

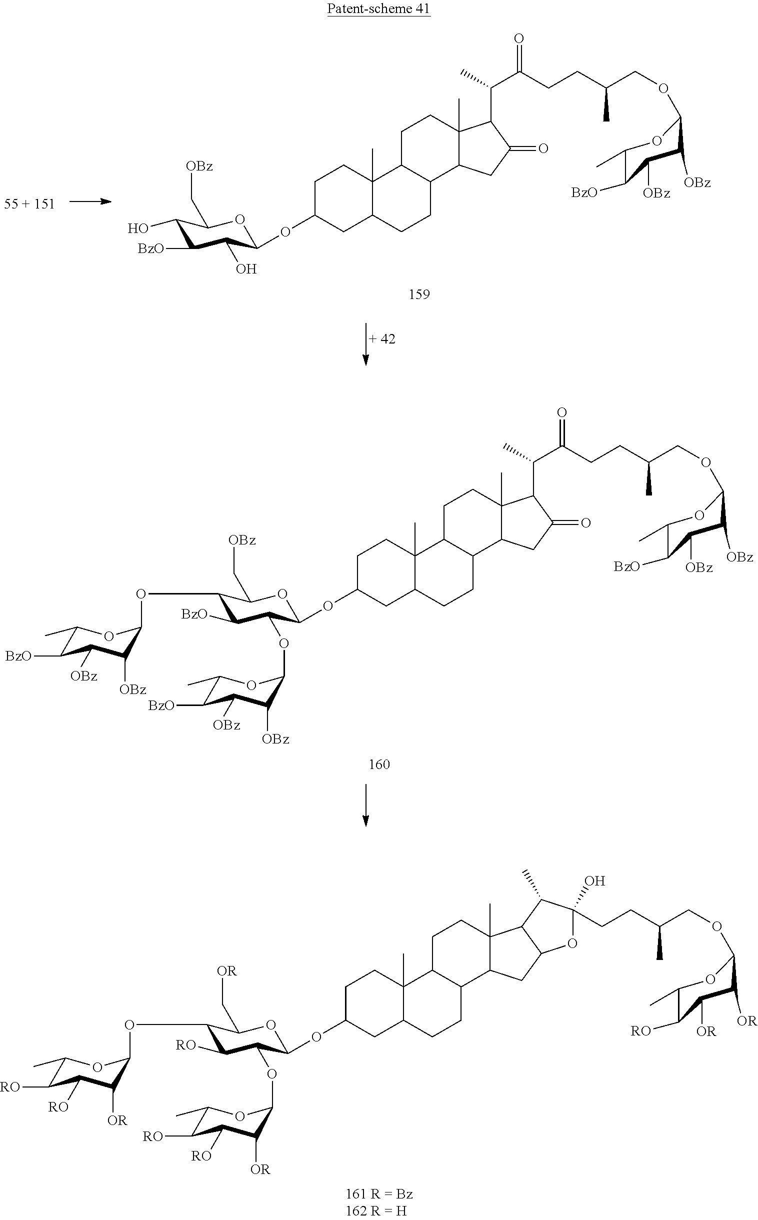

### Preparation of (159)

To a mixture of 55 (996 mg, 2.30 mmol) and 151 (1.71 g, 1.92 mmol) in anhyd CH₂Cl₂ (20 mL) at −40° C. was added NIS (517 mg, 2.30 mmol) and Me₃SiOTf (42 μL, 0.23 mmol) under N₂ atmosphere at −20° C. The mixture was stirred under these conditions for 40 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 159 as a foamy solid (1.94 g, 80%). MALDITOF-MS: Calcd for C₇₄H₈₄O₁₈: 1260.57 [M]⁺. Found 1283.70 [M+Na]⁺.

### Preparation of (160)

To a mixture of 159 (1.83 g, 1.45 mmol) and 42 (2.16 g, 3.48 mmol) in anhyd CH₂Cl₂ (30 mL) at −20° C. was added Me₃SiOTf (62 pt, 0.34 mmol) under N₂ atmosphere at −20° C. The mixture was stirred under these conditions for 30 min, at the end of which time TLC (petroleum ether-EtOAc, 3:1) indicated that all starting materials were consumed. The reaction mixture was neutralized with TEA, then concentrated. Column chromatography (petroleum ether-EtOAc, 2:1) of the residue gave 160 as a foamy solid (2.68 g, 85%). MALDITOF-MS: Calcd for C₁₂₈H₁₂₆O₃₂: 2176.84 [M]⁺. Found 2199.90 [M+Na]⁺.

### Preparation of (161)

The mixture of compound 160 (185 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 161 as a white solid (124 mg, 67%). MALDITOF-MS: Calcd for C₁₂₈H₁₃₀O₃₂: 2178.84 [M]⁺. Found 2201.90 [M+Na]⁺.

### Preparation of (162)

Compound 161 (120 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:1) indicated that the reaction was complete. The solution was neutralized with ion-exchange resin (H⁺), and then filtered and concentrated. The residue was purified by Bio-gel P₂ column to afford 125 as an amorphous solid (55 mg, 97%): Selected ¹H NMR (400 MHz, C₆D₅N): δ5.53 (d, 1H, J 2.5 Hz, H-1), 5.50 (d, 1H, J 2.6 Hz, H-1), 5.41 (d, 1H, J 2.6 Hz, H-1), 5.09 (d, 1H, J 7.2 Hz, H-1). ¹³C NMR (100 MHz, CD₅N): δ 105.5, 105.3, 104.8, 102.1. MALDITOF-MS: Calcd for C₅₁H₈₆O₂₁: 1034.57 [M]⁺. Found 1057.70 [M Na]⁺.

### Example 42

### The Preparation of (25S)-26-O-α-L-rhamnopyranosyl-22-hydroxy-5β-furostane-3β,26-diol-3-O-β-D-glucopyranosyl-(1→4)-β-D-glucopyranosyl-(1→4)-β-D-galactopyranoside (165)

The overall reaction scheme is as follows:

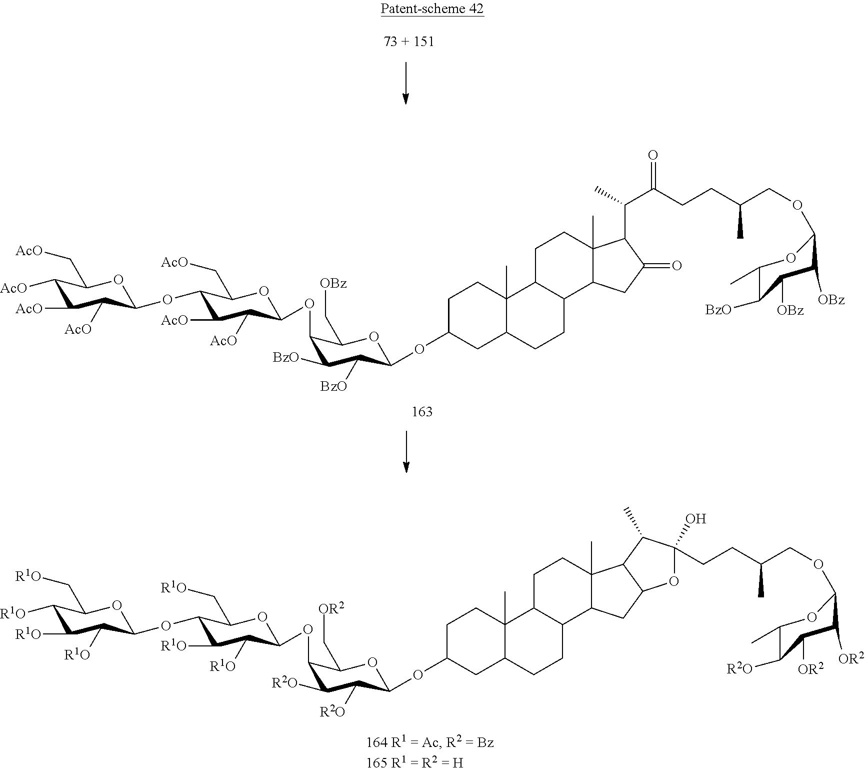

### Preparation of (163)

To a mixture of 73 (2.0 g, 1.7 mmol) and 151 (1.38 g, 1.55 mmol) in anhyd CH₂Cl₂ (20 mL) at −20° C. was added NIS (562 mg, 2.5 mmol) and Me₃SiOTf (31 μL, 0.17 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 163 as a foamy solid (2.55 g, 83%). MALDITOF-MS: Calcd for C₁₁₀₇H₁₂₂O₃₆: 1982.77 [M]⁺. Found 2005.90 [M+Na]⁺.

### Preparation of (164)

The mixture of compound 163 (168 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₃SO₄, and the solvent was removed under vacuum to give an colorless oil, which was purified by column chromatography (petroleum ether/ethyl acetate, 1:1) to give compound 164 as a white solid (113 mg, 67%). MALDITOF-MS: Calcd for C₁₁₀₇H₁₂₄O₃₆: 1984.77 [M]⁺. Found 2007.90 [M+Na]⁺.

### Preparation of (165)

Compound 164 (110 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 140 as an amorphous solid (56 mg, 96%): Selected ¹H NMR (400 MHz, C₆D₅N): δ5.53 (d, 1H, J 2.5 Hz, H-1), 5.25 (d, 1H, J 6.9 Hz, H-1), 5.21 (d, 1H, J 7.5 Hz, H-1), 5.04 (d, 1H, J 7.2 Hz, H-1). ¹³C NMR (100 MHz, CD₅N): δ 105.5, 103.1, 103.0, 102.6. MALDITOF-MS: Calcd for C₅₁H₈₆O₂₃: 1066.56 [M]⁺. Found 1089.70 [M+Na]⁺.

### Example 43

### The Preparation of (25S)-26-O-α-L-fucopyranosyl-22-hydroxy-5β-furostane-3β,26-diol-3-O-β-D-galactopyranoside (171)

The overall reaction scheme is as follows:

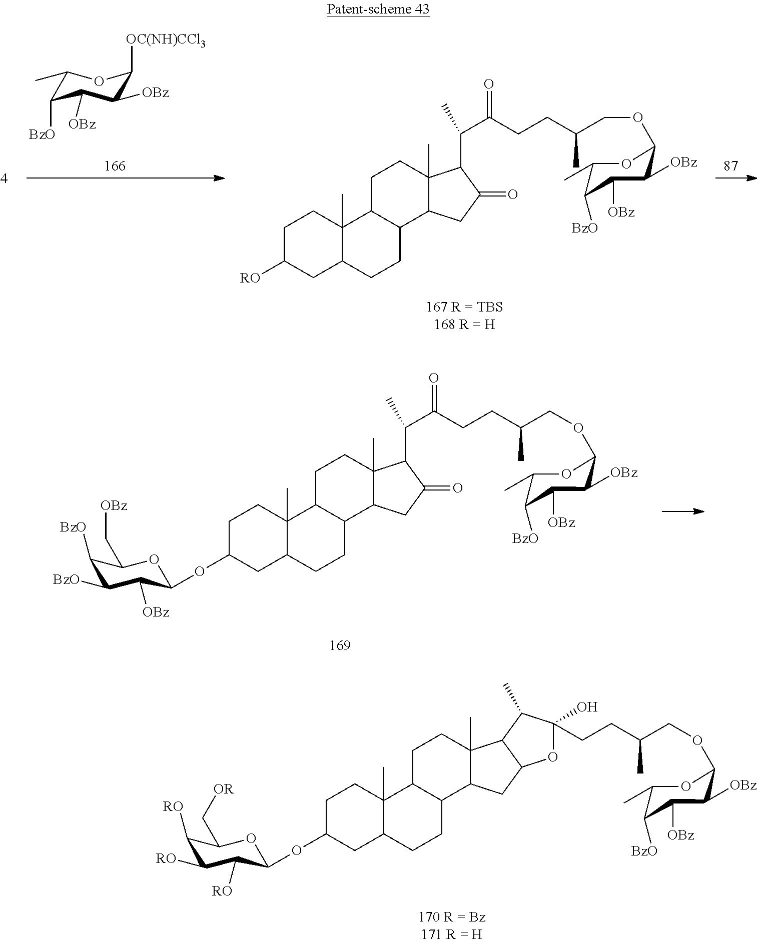

### Preparation of (168)

To a mixture of compounds 4 (2.25 g, 4.1 mmol) and 166 (2.98 g, 4.8 mmol, commercially available) in anhyd CH₂Cl₂ (36 mL) was added Me₃SiOTf (86 μL, 0.47 mmol) under N₂ atmosphere at −20° C. The mixture was stirred under these conditions for 40 min, at the end of which time TLC (petroleum ether-EtOAc, 4:1) indicated that all starting materials were consumed. The reaction mixture was neutralized with triethylamine (TEA), then concentrated. Column chromatography (petroleum ether-EtOAc, 6:1) of the residue gave 167 as a foamy solid (3.29 g, 80%).

To a solution of compound 167 (3.2 g, 3.2 mmol) in dry CH₂Cl₂ (40 mL) was added BF₃.Et₂O (1.0 mL, 7.3 mmol), and the mixture was stirred at room temperature for 4 h, and TLC (petroleum ether-EtOAc, 1:1) indicated that the reaction was complete. The mixture was diluted with CH₂Cl₂, washed with satd aq NaHCO₃ and then said aq NaCl. The organic layer was combined, dried, and concentrated. Purification by column chromatography (petroleum ether-EtOAc, 3:1) gave 168 as a white foamy solid (2.71 g, 95%): MALDITOF-MS: Calcd for C₅₄H₆₆O₁₁: 890.46 [M]⁺. Found 913.60 [M+Na]⁺

### Preparation of (169)

To a mixture of compound 87 (2.30 g, 3.10 mmol) and 168 (2.32 g, 2.6 mmol) in anhyd CH₂Cl₂ (50 mL) was added Me₃SiOTf (56 μL, 0.31 mmol) under N₂ atmosphere at 0° C. The mixture was stirred under r.t. for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 169 as a foamy solid (3.44 g, 90%): MALDITOF-MS: Calcd for C₈₈H₉₂O₂₀: 1468.62 [M]⁺. Found 1491.75 [M+Na]⁺.

### Preparation of (170)

The mixture of compound 169 (558 mg, 0.38 mmol) and NaBH₄ (357 mg, 9.4 mmol) in 2-propanol (16 mL) and CH₂Cl₂ (2 mL) was stirred at room temperature for about 7.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 170 as a white solid (363 mg, 65%): MALDITOF-MS: Calcd for C₈₈H₉₄O₂₀: 1470.62 [M]⁺. Found 1493.75 [M+Na]⁺.

### Preparation of (171)

Compound 170 (190 mg, 0.13 mmol) was dissolved in anhyd CH₂Cl₂-MeOH (1:2, 24 mL), and then 1.0 M NaOMe in MeOH (0.25 mL) was added at 0° C. After stirring at room temperature for 5 h, TLC (n-BuOH-EtOH—H₂O, 2:0.5:0.5) indicated that the reaction was complete. The solution was neutralized with ion-exchange resin (H⁺), and then filtered and concentrated. The residue was purified by Bio-gel P₂ column to afford 171 as an amorphous solid (92 mg, 95%): Selected ¹H NMR (400 MHz, C₆D₅N): δ5.51 (d, 1H, J 2.5 Hz, H-1), 5.21 (d, 1H, J 6.9 Hz, H-1). ¹³C NMR (100 MHz, CD₅N): δ105.5, 103.0. MALDITOF-MS: Calcd for C₃₉H₆₆O₁₃: 742.45 [M]⁴. Found 765.50 [M+Na]⁺.

### Example 44

### The Preparation of (25S)-26-O-α-L-fucopyranosyl-22-hydroxy-5β-furostane-3β,26-diol-3-O-β-D-glucopyranosyl-(1→2)-β-D-galactopyranoside (175)

The overall reaction scheme is as follows:

20

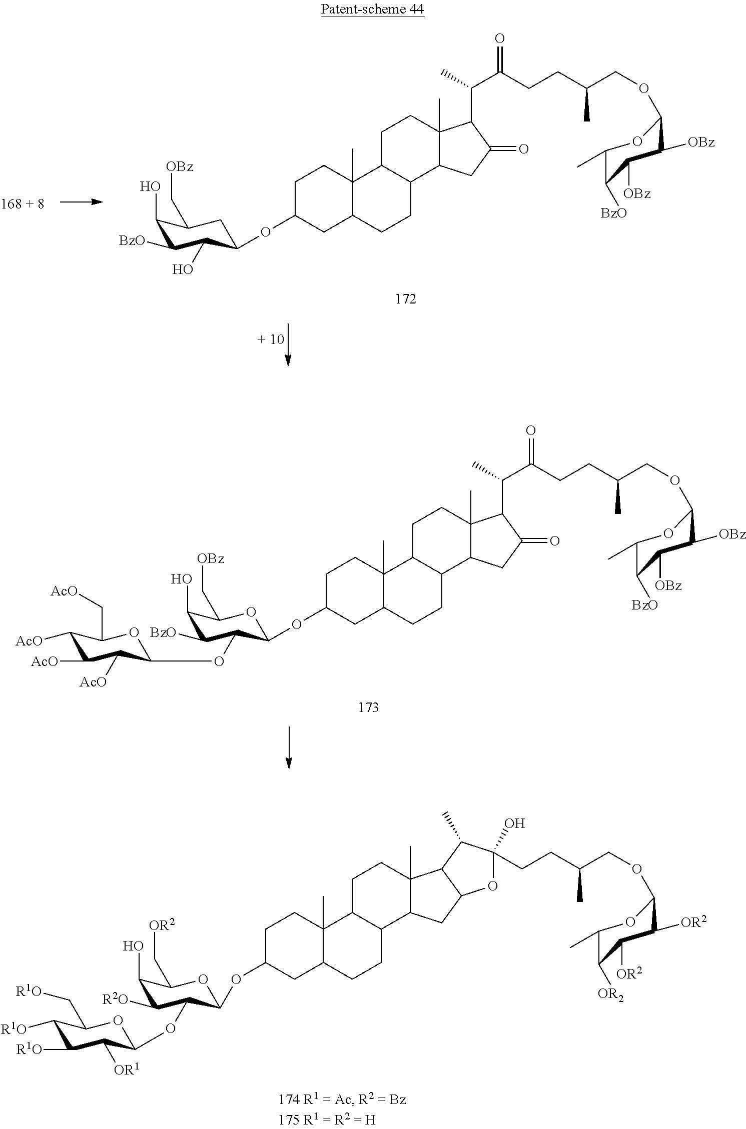

### Preparation of (172)

To a mixture of compound 8 (1.38 g, 3.08 mmol) and 168 (2.3 g, 2.6 mmol) in anhyd CH₂Cl₂ (50 mL) was added NIS (692 mg, 3.08 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 172 as a foamy solid (2.69 g, 82%): MALDITOF-MS: Calcd for C₇₄H₈₄O₁₈: 1260.57 [M]⁺. Found 1283.70 [M Na]⁺.

### Preparation of (173)

To a mixture of compound 10 (685 mg, 1.39 mmol) and 172 (1.45 g, 1.15 mmol) in anhyd CH₂Cl₂ (15 mL) was added Me₃SiOTf (26 μL, 0.14 mmol) under N₂ atmosphere at −42° C. The mixture was stirred under these conditions for 30 min, at which time TLC (petroleum ether-EtOAc, 1:1) indicated that all starting materials were consumed. The reaction mixture was neutralized with TEA, Then he mixture was concentrated with toluene and purified by column chromatography (petroleum ether-EtOAc, 3:1) to give 173 as a white foamy solid (1.37 g, 75%): MALDITOF-MS: Calcd for C₈₈H₁₀₂O₂₇: 1590.66 [M]⁺. Found 1613.75 [M+Na]⁺.

### Preparation of (174)

The mixture of compound 173 (135 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 174 as a white solid (88 mg, 65%): MALDITOF-MS: Calcd for C₈₈H₁₀₄O₂₇: 1592.66 [M]⁺. Found 1615.75 [M+Na]⁺.

### Preparation of (175)

Compound 174 (80 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 175 as an amorphous solid (43 mg, 95%): Selected ¹H NMR (400 MHz, C₆D₅N): δ5.53H, J 2.5 Hz, H-1), 5.23 (d, 1H, J 7.5 Hz, H-1), 5.11 (d, 1H, J 7.2 Hz, H-1). ¹³C NMR (100 MHz, CD₅N): δ105.5, 103.0, 102.4, MALDITOF-MS: Calcd for O₄₅H₇₆O₁₈: 904.50 [M]⁺. Found 927.75 [M+Na]⁺.

### Example 45

### The Preparation of (25S)-26-O-α-L-fucopyranosyl-22-hydroxy-5β-furostane-3β,26-diol-3-O-[α-L-rhamnopyranosyl-(1→4)]-[α-L-rhamnopyranosyl-(1→2)]-β-D-glucopyranoside (179)

The overall reaction scheme is as follows:

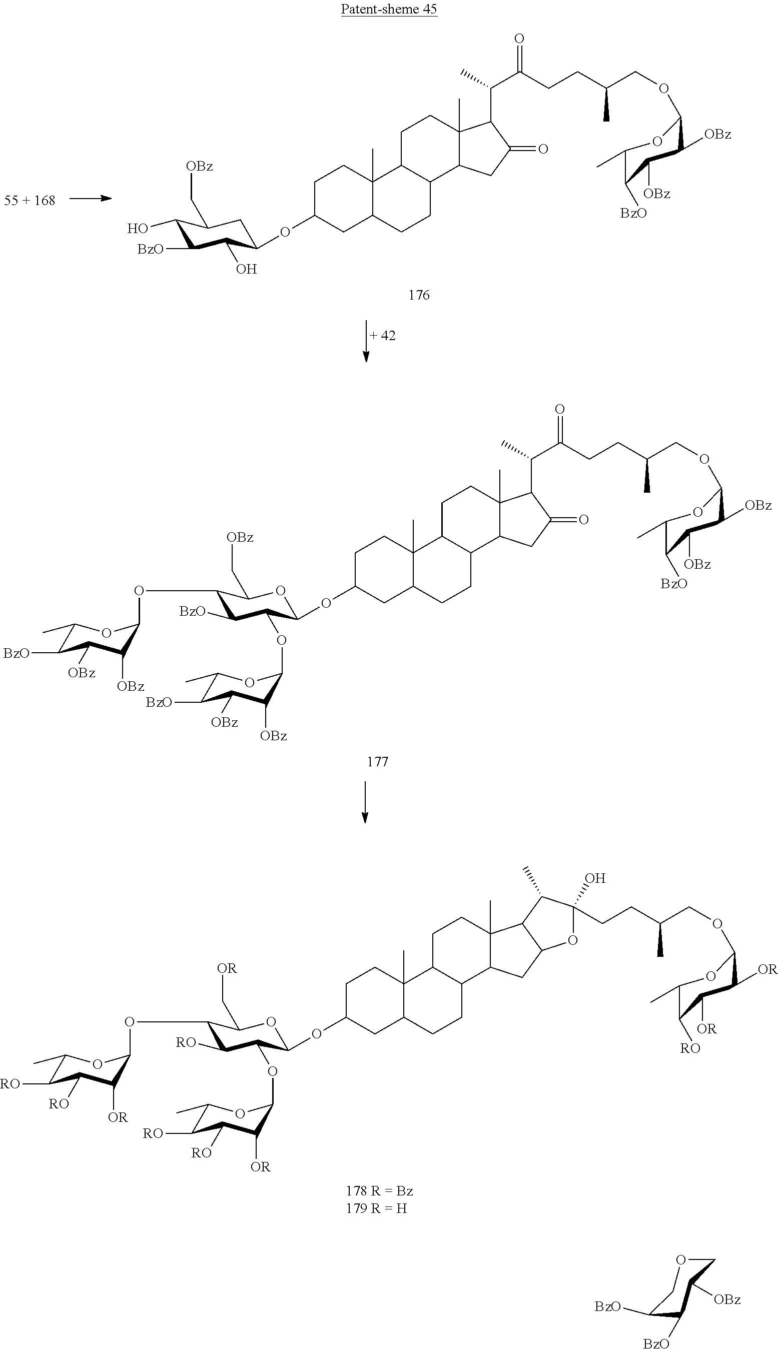

### Preparation of (176)

To a mixture of 55 (996 mg, 2.30 mmol) and 168 (1.71 g, 1.92 mmol) in anhyd CH₂Cl₂ (20 mL) at −40° C. was added NIS (517 mg, 2.30 mmol) and Me₃SiOTf (42 μL, 0.23 mmol) under N₂ atmosphere at −20° C. The mixture was stirred under these conditions for 40 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 176 as a foamy solid (1.94 g, 80%). MALDITOF-MS: Calcd for C₇₄H₈₄O₁₈: 1260.57 [M]⁺. Found 1283.70 [M+Na]⁺.

### Preparation of (177)

To a mixture of 176 (1.83 g, 1.45 mmol) and 42 (2.16 g, 3.48 mmol) in anhyd CH₂Cl₂ (30 mL) at −20° C. was added Me₃SiOTf (62 μL, 0.34 mmol) under N₂ atmosphere at −20° C. The mixture was stirred under these conditions for 30 min, at the end of which time TLC (petroleum ether-EtOAc, 3:1) indicated that all starting materials were consumed. The reaction mixture was neutralized with TEA, then concentrated. Column chromatography (petroleum ether-EtOAc, 2:1) of the residue gave 177 as a foamy solid (2.68 g, 85%). MALDITOF-MS: Calcd for C₁₇₈H₁₂₈O₃₂: 2176.84 [M]⁺. Found 2199.90 [M+Na]⁺.

### Preparation of (178)

The mixture of compound 177 (185 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 ml×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 178 as a white solid (124 mg, 67%). MALDITOF-MS: Calcd for C₁₂₈H₁₃₀O₃₂: 2178.84 [M]⁺. Found 2201.90 [M+Na]⁺.

### Preparation of (179)

Compound 178 (120 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:1) indicated that the reaction was complete. The solution was neutralized with ion-exchange resin (H⁺), and then filtered and concentrated. The residue was purified by Bio-gel P₂ column to afford 179 as an amorphous solid (55 mg, 97%): Selected ¹H NMR (400 MHz, C₆D₅N): δ5.53 (d, 1H, J 2.5 Hz, H-1), 5.50 (d, 1H, J 2.6 Hz, H-1), 5.47 (d, 1H, J 2.5 Hz, H-1), 5.09 (d, 1H, J 7.2 Hz, H-1). ¹³C NMR (100 MHz, CD₅N): δ 105.5, 105.2, 104.1, 102.5.

MALDITOF-MS: Calcd for C₅₁H₈₆O₂₁: 1034.57 [M]⁺. Found 1057.70 [M+Na]⁺.

### Example 46

### The Preparation of (25S)-26-O-α-L-fucopyranosyl-22-hydroxy-5β-furostane-313,26-diol-3-O-β-D-glucopyranosyl-(1→4)-β-D-glucopyranosyl-(1→4)-β-D-galactopyranoside (182)

The overall reaction scheme is as follows:

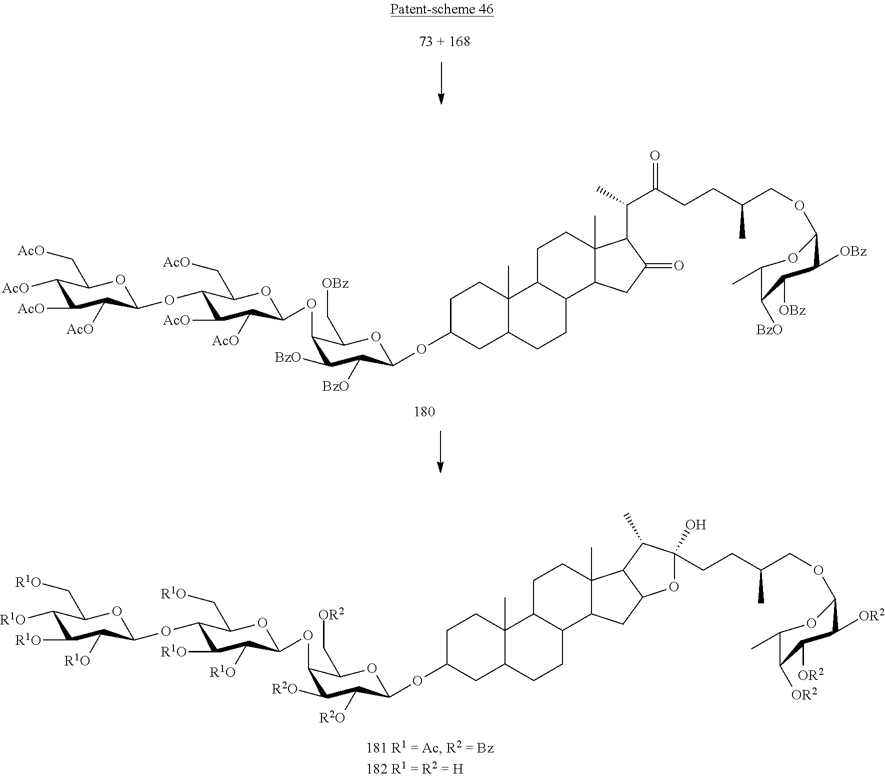

### Preparation of (180)

To a mixture of 73 (2.0 g, 1.7 mmol) and 168 (1.38 g, 1.55 mmol) in anhyd CH₂Cl₂ (20 mL) at −20° C. was added NIS (562 mg, 2.5 mmol) and Me₃SiOTf (31 μL, 0.17 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 180 as a foamy solid (2.55 g, 83%). MALDITOF-MS: Calcd for C₁₀₇H₁₂₂O₃₆: 1982.77 [M]⁺. Found 2005.90 [M Na]⁺,

### Preparation of (181)

The mixture of compound 180 (168 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 181 as a white solid (113 mg, 67%). MALDITOF-MS: Calcd for C₁₀₇H₁₂₄O₃₆: 1984.77 [M]⁺. Found 2007.90 [M+Na]⁺.

### Preparation of (182)

Compound 181 (110 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 182 as an amorphous solid (56 mg, 96%): Selected ¹H NMR (400 MHz, C₆D₅N): δ5.53 (d, 1H, J 2.5 Hz, H-1), 5.28 (d, 1H, J 6.9 Hz, H-1), 5.25 (d, 1H, J 7.5 Hz, H-1), 5.09 (d, 1H, J 7.2 Hz, H-1). ¹³C NMR (100 MHz, CD₅N): δ 105.5, 103.2, 103.0, 102.5. MALDITOF-MS: Calcd for C₅₁H₈₆O₂₃: 1066.56 [M]⁺. Found 1089.70 [M+Na]⁺.

### Example 47

### The Preparation of (25S)-26-O-α-L-arabinopyranosyl-22-hydroxy-5β-furostane-3β,26-diol-3-O-β-D-galactopyranoside (188)

The overall reaction scheme is as follows:

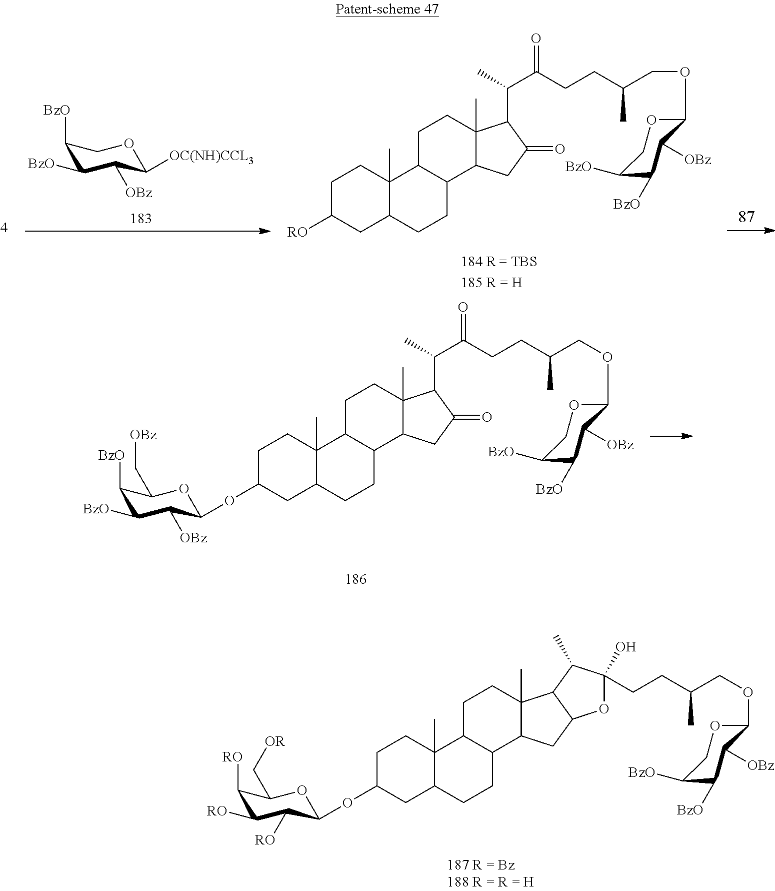

### Preparation of (185)

To a mixture of compounds 4 (2.25 g, 4.1 mmol) and 183 (2.91 g, 4.8 mmol, commercially available) in anhyd CH₂Cl₂ (36 mL) was added Me₃SiOTf (86 μL, 0.47 mmol) under N₂ atmosphere at −20° C. The mixture was stirred under these conditions for 40 min, at the end of which time TLC (petroleum ether-EtOAc, 4:1) indicated that all starting materials were consumed. The reaction mixture was neutralized with triethylamine (TEA), then concentrated. Column chromatography (petroleum ether-EtOAc, 6:1) of the residue gave 184 as a foamy solid (3.45 g, 85%).

To a solution of compound 184 (3.2 g, 3.2 mmol) in dry CH₂Cl₂ (40 mL) was added BF₃.Et₂O (1.0 mL, 7.3 mmol), and the mixture was stirred at room temperature for 4 h, and TLC (petroleum ether-EtOAc, 1:1) indicated that the reaction was complete. The mixture was diluted with CH₂Cl₂, washed with satd aq NaHCO₃ and then satd aq NaCl. The organic layer was combined, dried, and concentrated. Purification by column chromatography (petroleum ether-EtOAc, 3:1) gave 185 as a white foamy solid (2.67 g, 95%): MALDITOF-MS: Calcd for C₅₃H⁶⁴O₁₁: 876.44 [M]⁺. Found 899.50 [M+Na]⁺

### Preparation of (186)

To a mixture of compound 87 (2.30 g, 3.10 mmol) and 186 (2.28 g, 2.6 mmol) in anhyd CH₂Cl₂ (50 mL) was added Me₃SiOTf (56 μL, 0.31 mmol) under 1\1, atmosphere at 0° C. The mixture was stirred under r.t. for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 186 as a foamy solid (3.41 g, 90%): MALDITOF-MS: Calcd for C₈₇H₉₀O₂₀: 1454.60 [M]⁺. Found 1477.75 [M+Na]⁺.

### Preparation of (187)

The mixture of compound 186 (553 mg, 0.38 mmol) and NaBH₄ (357 mg, 9.4 mmol) in 2-propanol (16 mL) and CH₂Cl₂ (2 mL) was stirred at room temperature for about 7.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 187 as a white solid (360 mg, 65%): MALDITOF-MS: Calcd for C₈₂H₉₂O₂₀: 1456.60 [M]⁺. Found 1479.75 [M+Na]⁺.

### Preparation of (188)

Compound 187 (189 mg, 0.13 mmol) was dissolved in anhyd CH₂Cl₂-MeOH (1:2, 24 mL), and then 1.0 M NaOMe in MeOH (0.25 mL) was added at 0° C. After stirring at room temperature for 5 h, TLC (n-BuOH-EtOH—H₂O, 2:0.5:0.5) indicated that the reaction was complete. The solution was neutralized with ion-exchange resin (H⁺), and then filtered and concentrated. The residue was purified by Bio-gel P₂ column to afford 188 as an amorphous solid (91 mg, 95%): Selected ¹H NMR (400 MHz, C₆D₅N): δ5.25 (d, 1H, J 7.5 Hz, H-1), 5.11 (d, 1H, J 7.2 Hz, H-1), ¹³C NMR (100 MHz, CD₅N): δ105.4, 102.5, MALDITOF-MS: Calcd for C₃₈H₆₄O₁₃: 728.43 [M]⁺. Found 751.50 [M+Na]⁺.

### Example 48

### The Preparation of (25S)-26-O-α-L-arabinopyranosyl-22-hydroxy-5β-furostane-3β,26-diol-3-O-β-D-glucopyranosyl-(1→2)-β-D-galactopyranoside (192)

The overall reaction scheme is as follows

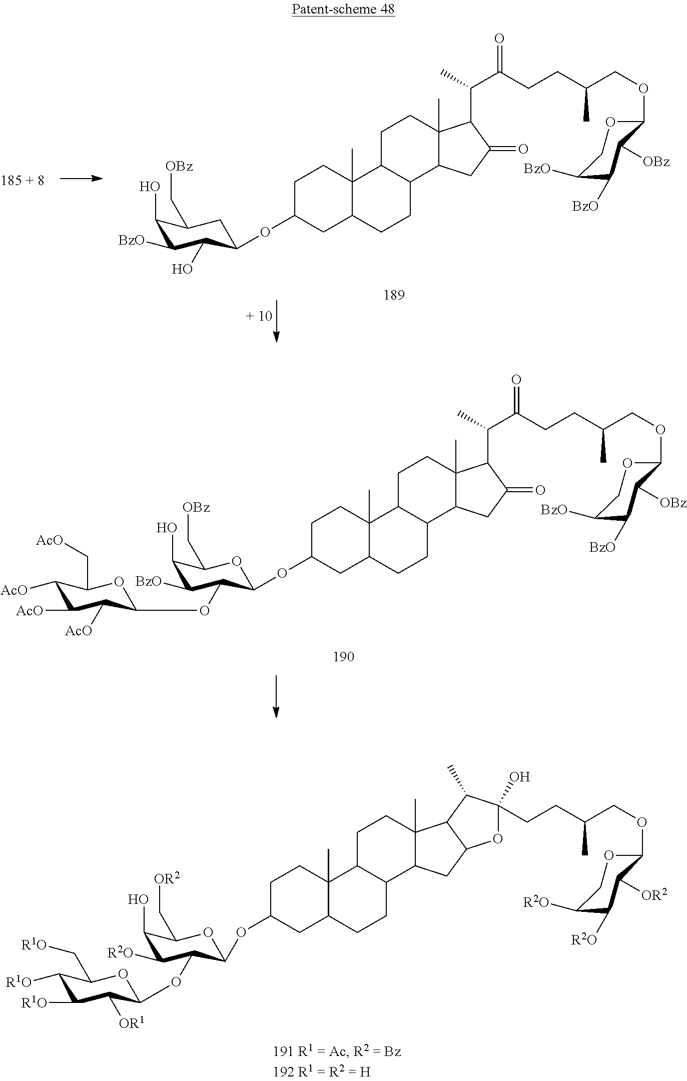

### Preparation of (190)

To a mixture of compound 8 (1.38 g, 3.08 mmol) and 185 (2.28 g, 2.6 mmol) in anhyd CH₂Cl₂ (50 mL) was added NIS (692 mg, 3.08 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 189 as a foamy solid (2.68 g, 82%): MALDITOF-MS: Calcd for C₇₃H₈₂O₁₈: 1246.55 [M]⁺. Found 1269.70 [M+Na]⁺.

### Preparation of (190)

To a mixture of compound 10 (685 mg, 1.39 mmol) and 189 (1.43 g, 1.15 morel) in anhyd CH₂Cl₂ (15 mL) was added Me₃SiOTf (26 μL, 0.14 mmol) under N₂ atmosphere at −42° C. The mixture was stirred under these conditions for 30 min, at which time TLC (petroleum ether-EtOAc, 1:1) indicated that all starting materials were consumed. The reaction mixture was neutralized with TEA, Then he mixture was concentrated with toluene and purified by column chromatography (petroleum ether-EtOAc, 3:1) to give 190 as a white foamy solid (1.35 g, 75%): MALDITOF-MS: Calcd for C₈₇H₁₀₀O₂₇: 1576.65 [M]⁺. Found 1599.80 [M+Na]⁺.

### Preparation of (191)

The mixture of compound 190 (134 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 191 as a white solid (85 mg, 65%): MALDITOF-MS: Calcd for C₈₇H₁₀₂O₂₇: 1578.65 [M]⁺. Found 1601.80 [M+Na]⁺.

### Preparation of (192)

Compound 191 (79 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 192 as an amorphous solid (41 mg, 95%): Selected ¹H NMR (400 MHz, C₆D₅N): δ5.40 (d, 1H, J 6.7 Hz, H-1), 5.22 (d, 1H, J 7.2 Hz, H-1), 5.10 (d, 1H, J 7.5 Hz, H-1). ¹³C NMR (100 MHz, CD₅N): δ105.5, 103.8, 103.0. MALDITOF-MS: Calcd for C₄₄H₇₄O₁₈: 890.49 [M]⁺. Found 913.55 [M+Na]⁺.

### Example 49

### The Preparation of (25S)-26-O-α-L-arabinopyranosyl-22-hydroxy-5β-furostane-3β,26-diol-3-O-[α-L-rhamnopyranosyl-(1→4)]-[α-L-rhamnopyranosyl-(1→2)]-β-D-glucopyranoside (196)

The overall reaction scheme is as follows:

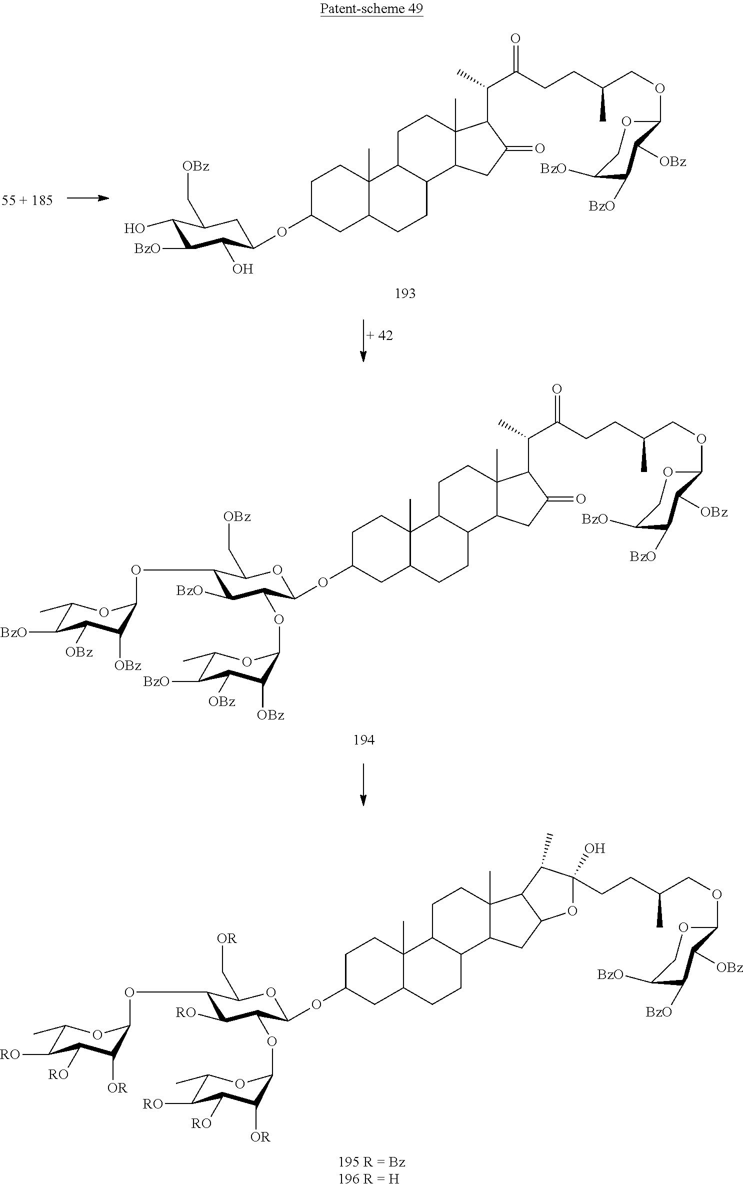

### Preparation of (193)

To a mixture of 55 (996 mg, 2.30 mmol) and 185 (1.68 g, 1.92 mmol) in anhyd CH₂Cl₂ (20 mL) at −40° C. was added NIS (517 mg, 2.30 mmol) and Me₃SiOTf (42 μL, 0.23 mmol) under N₂ atmosphere at −20° C. The mixture was stirred under these conditions for 40 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 193 as a foamy solid (1.91 g, 80%). MALDITOF-MS: Calcd for C₇₃H₈₂O₁₈: 1246.55 [M]⁺. Found 1269.70 [M Na]⁺.

### Preparation of (194)

To a mixture of 193 (1.80 g, 1.45 mmol) and 42 (2.16 g, 3.48 mmol) in anhyd CH₂Cl₂ (30 mL) at −20° C. was added Me₃SiOTf (62 μL, 0.34 mmol) under N₂ atmosphere at −20° C. The mixture was stirred under these conditions for 30 min, at the end of which time TLC (petroleum ether-EtOAc, 3:1) indicated that all starting materials were consumed. The reaction mixture was neutralized with TEA, then concentrated. Column chromatography (petroleum ether-EtOAc, 2:1) of the residue gave 194 as a foamy solid (2.65 g, 85%). MALDITOF-MS: Calcd for C₁₂₇H₁₂₆O₃₂: 2162.82 [M]⁺. Found 2185.90 [M+Na]⁺.

### Preparation of (195)

The mixture of compound 194 (182 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 195 as a white solid (121 mg, 67%). MALDITOF-MS: Calcd for C₁₂₇H₁₂₈O₃₂: 2164.82 [M]⁺. Found 2187.90 [M+Na]⁺.

### Preparation of (196)

Compound 195 (118 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:1) indicated that the reaction was complete. The solution was neutralized with ion-exchange resin (H⁺), and then filtered and concentrated. The residue was purified by Bio-gel P₂ column to afford 196 as an amorphous solid (52 mg, 97%): Selected ¹H NMR (400 MHz, C₆D₅N): δ5.53 (d, 1H, J 2.5 Hz, H-1), 5.49 (d, 1H, J 2.6 Hz, H-1), 5.40 (d, 1H, J 6.5 Hz, H-1), 5.09 (d, 1H, J 7.2 Hz, H-1). ¹³C NMR (100 MHz, CD₅N): δ 105.5, 105.3, 104.4, 1021. MALDITOF-MS: Calcd for C₅₀H₈₄O₂₁: 1020.55 [M]⁺. Found 1043.70 [M+Na]⁺.

### Example 50

### The Preparation of (25S)-26-O-α-L-arabinopyranosyl-22-hydroxy-5β-furostane-3β,26-diol-3-O-β-D-glucopyranosyl-(1→4)-β-D-glucopyranosyl-(1→4)-β-D-galactopyranoside (199)

The overall reaction scheme is as follows:

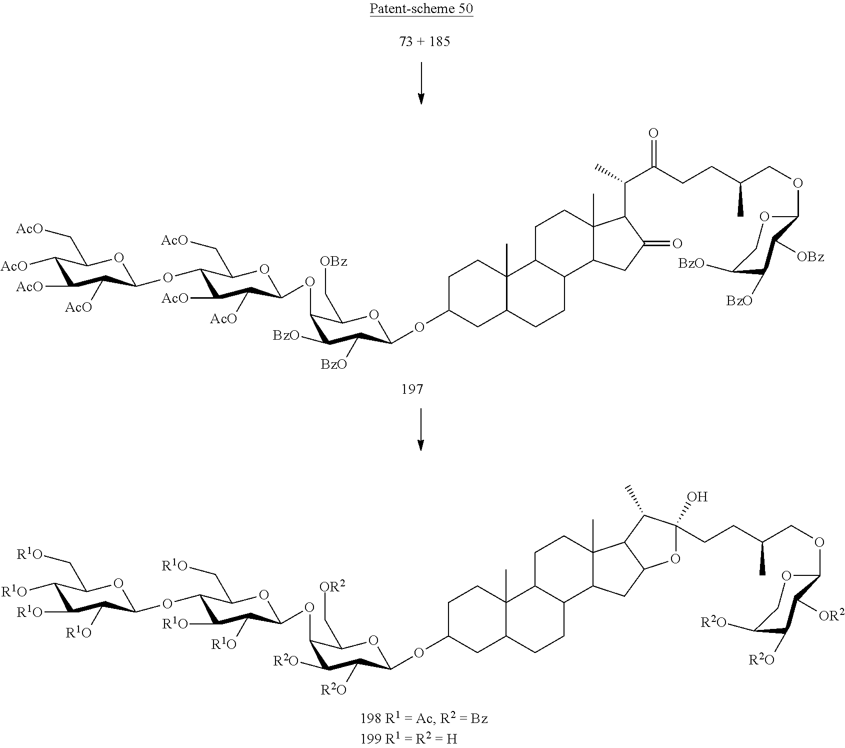

### Preparation of (197)

To a mixture of 73 (2.0 g, 1.7 mmol) and 185 (1.35 g, 1.55 mmol) in anhyd CH₂Cl₂ (20 mL) at −20° C. was added NIS (562 mg, 2.5 mmol) and Me₃SiOTf (31 μL, 0.17 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 197 as a foamy solid (2.52 g, 83%). MALDITOF-MS: Calcd for O₁₀₆H₁₂₀O₃₆: 1968.76 [M]⁺. Found 1991.85 [M+Na]⁺.

### Preparation of (198)

The mixture of compound 197 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 198 as a white solid (111 mg, 67%). MALDITOF-MS: Calcd for C₁₀₆H₁₂₂O₃₆: 1970.76 [M]⁺. Found 1993.85 [M+Na]⁺.

### Preparation of (199)

Compound 198 (110 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 199 as an amorphous solid (54 mg, 96%): Selected ¹H NMR (400 MHz, C₆D₅N): δ5.30 (d, 1H, J 7.5 Hz, H-1), 5.28 (d, 1H, J 6.1 Hz, H-1), 5.25 (d, 1H, J 7.2 Hz, H-1), 5.09 (d, 1H, J 7.7 Hz, H-1). ¹³C NMR (100 MHz, CD₅N): δ 105.5, 104.8, 103.4, 102.7. MALDITOF-MS: Calcd for C₅₀H₈₄O₂₃: 1052.54 [M]⁺. Found 1075.70 [M+Na]⁺.

### Example 51

### The Preparation of (25S)-26-O-β-D-xylopyranosyl-22-hydroxy-5β-furostane-3β,26-diol-3-O-β-D-galactopyranoside (205)

The overall reaction scheme is as follows:

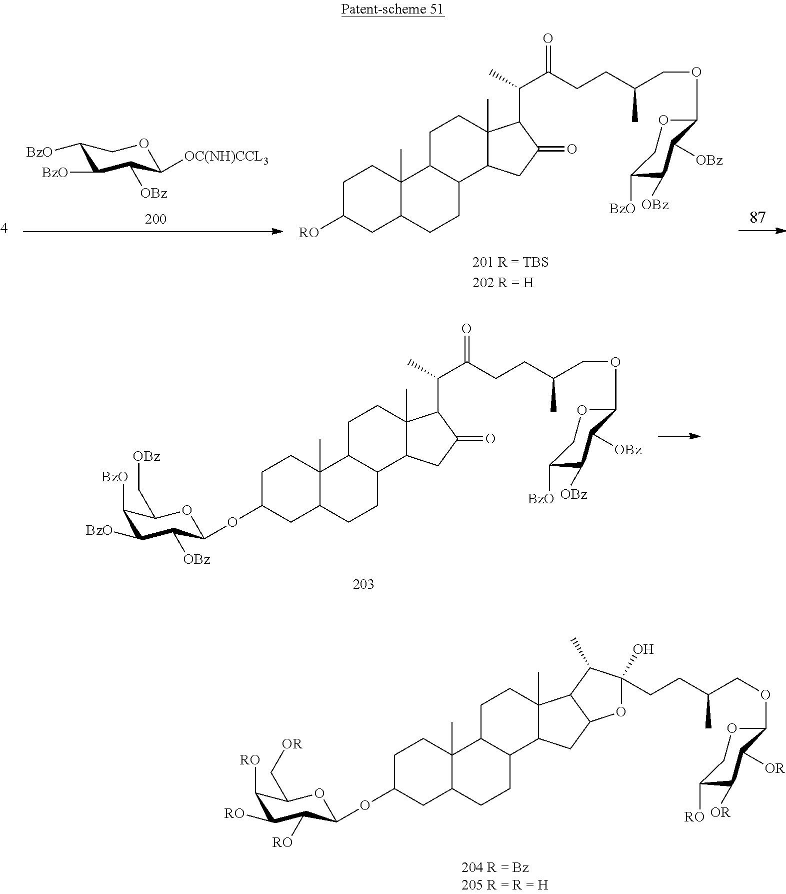

### Preparation of (202)

To a mixture of compounds 4 (2.25 g, 4.1 mmol) and 200 (2.91 g, 4.8 mmol, commercially available) in anhyd CH₂Cl₂ (36 mL) was added Me₃SiOTf (86 μL, 0.47 mmol) under N₂ atmosphere at −20° C. The mixture was stirred under these conditions for 40 min, at the end of which time TLC (petroleum ether-EtOAc, 4:1) indicated that all starting materials were consumed. The reaction mixture was neutralized with triethylamine (TEA), then concentrated. Column chromatography (petroleum ether-EtOAc, 6:1) of the residue gave 201 as a foamy solid (3.45 g, 85%).

To a solution of compound 201 (3.2 g, 3.2 mmol) in dry CH₂Cl₂ (40 mL) was added BF₃.Et₂O (1.0 mL, 7.3 mmol), and the mixture was stirred at room temperature for 4 h, and TLC (petroleum ether-EtOAc, 1:1) indicated that the reaction was complete. The mixture was diluted with CH₂Cl₂, washed with satd aq NaHCO₃ and then satd aq NaCl. The organic layer was combined, dried, and concentrated. Purification by column chromatography (petroleum ether-EtOAc, 3:1) gave 202 as a white foamy solid (2.67 g, 95%): MALDITOF-MS: Calcd for C₅₃H₆₄O₁₁: 876.44 [M]⁺. Found 899.50 [M+Na]⁺

### Preparation of (203)

To a mixture of compound 87 (2.30 g, 3.10 mmol) and 202 (2.28 g, 2.6 mmol) in anhyd CH₂Cl₂ (50 mL) was added Me₃SiOTf (56 μL, 0.31 mmol) under N₂ atmosphere at 0° C. The mixture was stirred under r.t. for 30 min, at the end of which time TLC (petroleum ether-EtOAc, 2:1) indicated that all starting materials were consumed. The reaction mixture was neutralized with TEA, then concentrated. Column chromatography (petroleum ether-EtOAc, 2:1) of the residue gave 203 as a foamy solid (3.41 g, 90%): MALDITOF-MS: Calcd for C₈₇H₉₀O₂₀: 1454.60 [M]⁺. Found 1477.75 [M+Na]⁺.

### Preparation of (204)

The mixture of compound 203 (553 mg, 0.38 mmol) and NaBH₄ (357 mg, 9.4 mmol) in 2-propanol (16 mL) and CH₂Cl₂ (2 mL) was stirred at room temperature for about 7.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 204 as a white solid (360 mg, 65%): MALDITOF-MS: Calcd for C₈₇H₉₂O₂₀: 1456.60 [M]⁺. Found 1479.75 [M+Na]⁺.

### Preparation of (205)

Compound 204 (189 mg, 0.13 mmol) was dissolved in anhyd CH₂Cl₂-MeOH (1:2, 24 mL), and then 1.0 M NaOMe in MeOH (0.25 mL) was added at 0° C. After stirring at room temperature for 5 μl, TLC (n-BuOH-EtOH—H₂O, 2:0.5:0.5) indicated that the reaction was complete. The solution was neutralized with ion-exchange resin (H⁺), and then filtered and concentrated. The residue was purified by Bio-gel P₂ column to afford 205 as an amorphous solid (91 mg, 95%): Selected ¹H NMR (400 MHz, C₆D₅N): δ5.25 (d, 1H, J 6.9 Hz, H-1), 5.10 (d, 1H, J 7.5 Hz, H-1). ¹³C NMR (100 MHz, CD₅N): δ105.5, 103.4. MALDITOF-MS: Calcd for C₃₈H₆₄O₁₃: 728.43 [M]⁺. Found 751.50 [M+Na]⁺.

### Example 52

### The Preparation of (25S)-26-O-β-D-xylopyranosyl-22-hydroxy-5β-furostane-3β,26-diol-3-O-β-D-glucopyranosyl-(1→2)-β-D-galactopyranoside (209)

The overall reaction scheme is as follows

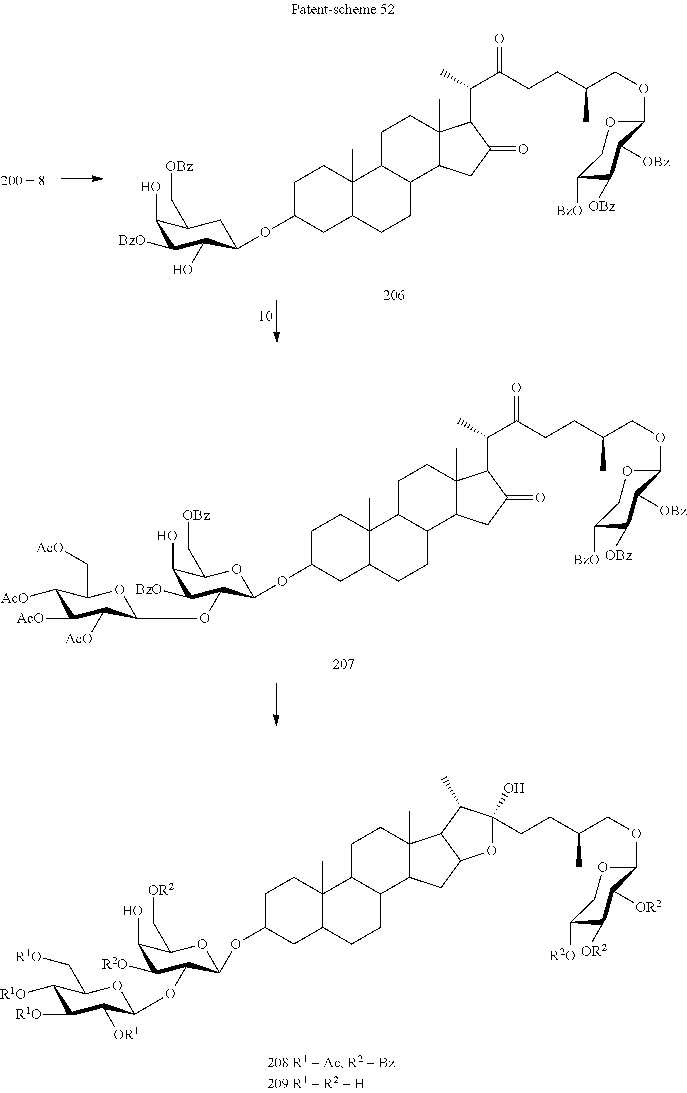

### Preparation of (206)

To a mixture of compound 8 (1.38 g, 3.08 mmol) and 202 (2.28 g, 2.6 mmol) in anhyd CH₂Cl₂ (50 mL) was added NIS (692 mg, 3.08 mmol) and Me₃SiOTf (55 μL, 0.30 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 206 as a foamy solid (2.68 g, 82%): MALDITOF-MS: Calcd for C₇₃H₈₂O₁₈: 1246.55 [M]⁺. Found 1269.70 [M+Na]⁺.

### Preparation of (207)

To a mixture of compound 10 (685 mg, 1.39 mmol) and 206 (1.43 g, 1.15 mmol) in anhyd CH₂Cl₂ (15 mL) was added Me₃SiOTf (26 μL, 0.14 mmol) under N₂ atmosphere at −42° C. The mixture was stirred under these conditions for 30 min, at which time TLC (petroleum ether-EtOAc, 1:1) indicated that all starting materials were consumed. The reaction mixture was neutralized with TEA, Then he mixture was concentrated with toluene and purified by column chromatography (petroleum ether-EtOAc, 3:1) to give 207 as a white foamy solid (1.35 g, 75%): MALDITOF-MS: Calcd for C₈₇H₁₀₀O₂₇: 1576.65 [M]⁺. Found 1599.80 [M+Na]⁺.

### Preparation of (208)

The mixture of compound 207 (134 mg, 0.085 mmol) and NaBH₄ (96 mg, 2.5 mmol) in 2-propanol (8 mL) and CH₂Cl₂ (1 mL) was stirred at room temperature for about 8.5 h, The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 208 as a white solid (85 mg, 65%): MALDITOF-MS; Calcd for C₈₇H₁₀₂O₂₇: 1578.65 [M]⁺. Found 1601.80 [M+Na]⁺.

### Preparation of (209)

Compound 208 (79 mg, 0.050 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 209 as an amorphous solid (41 mg, 95%): Selected ¹H NMR (400 MHz, C₆D₅N): δ5.28 (d, 1H, J 7.5 Hz, H-1), 5.24 (d, 1H, J 6.9 Hz, H-1), 5.10 (d, 1H, J 7.5 Hz, H-1). ¹³C NMR (100 MHz, CD₅N): δ105.5, 103.8, 103.0. MALDITOF-MS: Calcd for C₄₄H₇₄O₁₈: 890.49 [M]⁺. Found 913.55 [M+Na]⁺.

### Example 53

### The Preparation of (25S)-26-O-β-D-xylopyranosyl-22-hydroxy-5β-furostane-3β,26-diol-3-O-[α-L-rhamnopyranosyl-(1→4)]-[α-L-rhamnopyranosyl-(1→2)]-β-D-glucopyranoside (213)

The overall reaction scheme is as follows:

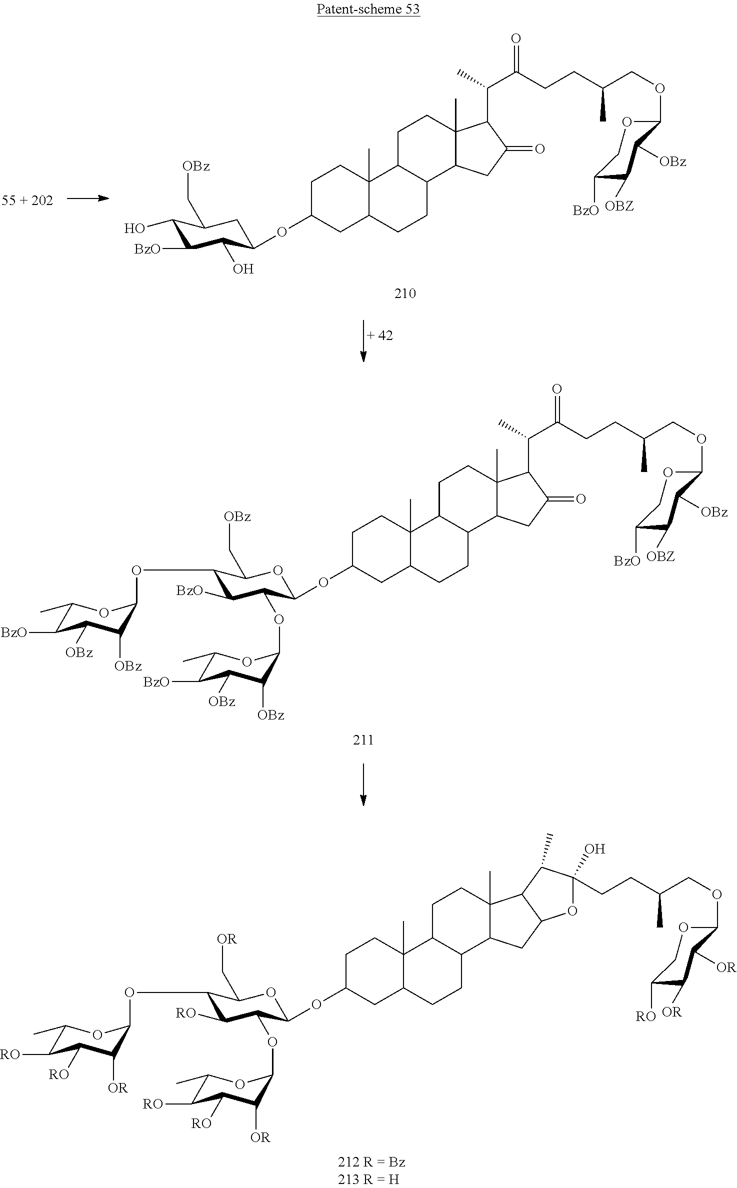

### Preparation of (210)

To a mixture of 55 (996 mg, 2.30 mmol) and 202 (1.68 g, 1.92 mmol) in anhyd CH₂Cl₂ (20 mL) at −40° C. was added NIS (517 mg, 2.30 mmol) and Me₃SiOTf (42 μL, 0.23 mmol) under N₂ atmosphere at −20° C. The mixture was stirred under these conditions for 40 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 210 as a foamy solid (1.91 g, 80%). MALDITOF-MS: Calcd for C73H82O18: 1246.55 [M]⁺. Found 1269.70 [M+Na]⁺.

### Preparation of (211)

To a mixture of 210 (1.80 g, 1.45 mmol) and 42 (2.16 g, 3.48 mmol) in anhyd CH₂Cl₂ (30 mL) at −20° C. was added Me₃SiOTf (62 μL, 0.34 mmol) under N₂ atmosphere at −20° C. The mixture was stirred under these conditions for 30 min, at the end of which time TLC (petroleum ether-EtOAc, 3:1) indicated that all starting materials were consumed. The reaction mixture was neutralized with TEA, then concentrated. Column chromatography (petroleum ether-EtOAc, 2:1) of the residue gave 211 as a foamy solid (2.65 g, 85%). MALDITOF-MS: Calcd for C₁₂₇H₁₂₆O₃₂: 2162.82 [M]⁺. Found 2185.90 [M+Na]⁺.

### Preparation of (212)

The mixture of compound 211 (182 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 mL×3) and dried over anhydrous Na₂SO₄, and the solvent was removed under vacuum to give an colorless oil, which was purified by column chromatography (petroleum ether/ethyl acetate, 1:1) to give compound 212 as a white solid (121 mg, 67%). MALDITOF-MS: Calcd for C₁₂₇H₁₂₈O₃₂: 2164.82 [M]⁺. Found 2187.90 [M+Na]⁺.

### Preparation of (213)

Compound 212 (118 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:1) indicated that the reaction was complete. The solution was neutralized with ion-exchange resin (H⁺), and then filtered and concentrated. The residue was purified by Bio-gel P₂ column to afford 213 as an amorphous solid (52 mg, 97%): Selected ¹H NMR (400 MHz, C₆D₅N): δ5.55 (d, 1H, J 2.5 Hz, H-1), 5.50 (d, 1H, J 2.9 Hz, H-1), 5.10 (d, 1H, J 7.5 Hz, H-1), 5.05 (d, 1H, J 7.2 Hz, H-1). ¹³C NMR (100 MHz, CD₅N): δ 105.5, 104.8, 103.2, 103.1. MALDITOF-MS: Calcd for C₅₀H₈₄O₂₁: 1020.55 [M]⁺. Found 1043.70 [M+Na]⁺.

### Example 54

### The Preparation of (25S)-26-O-β-D-xylopyranosyl-22-hydroxy-5β-furostane-3β,26-diol-3-O-β-D-glucopyranosyl-(1→4)-β-D-glucopyranosyl-(1→4)-β-D-galactopyranoside (216)

The overall reaction scheme is as follows:

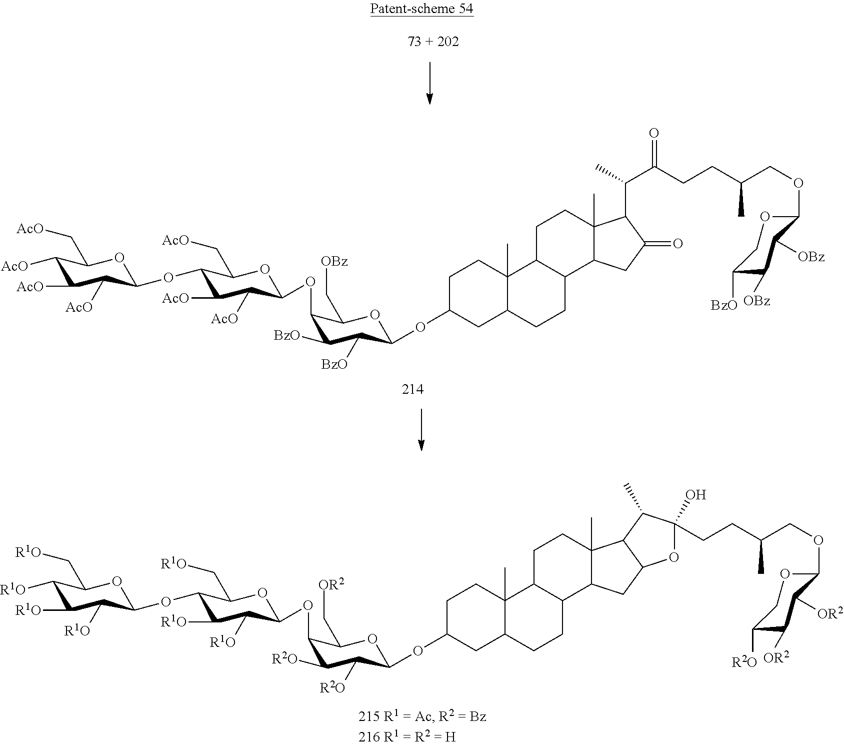

### Preparation of (214)

To a mixture of 73 (2.0 g, 1.7 mmol) and 202 (1.35 g, 1.55 mmol) in anhyd CH₂Cl₂ (20 mL) at −20° C. was added NIS (562 mg, 2.5 mmol) and Me₃SiOTf (31 μL, 0.17 mmol) under N₂ atmosphere at −20° C. The mixture was stirred under these conditions for 30 min, at the end of which time TLC (petroleum ether-EtOAc, 1:1) indicated that all starting materials were consumed. The reaction mixture was neutralized with TEA, then concentrated. Column chromatography (petroleum ether-EtOAc, 2:1) of the residue gave 214 as a foamy solid (2.52 g, 83%). MALDITOF-MS: Calcd for C₁₀₆H₁₂₀O₃₆: 1968.76 [M]⁺. Found 1991.85 [M+Na]⁺.

### Preparation of (215)

The mixture of compound 214 (165 mg, 0.085 mmol) and NaBH₄ (96 mg, 2.5 mmol) in 2-propanol (8 mL) and CH₂Cl₂ (1 mL) was stirred at room temperature for about 8.5 h. The reaction mixture was then extracted with CH₂Cl₂ (100 mL×2), the combined organic layer was washed with water (100 in L×3) and dried over anhydrous Na₂SO₄, and the solvent was removed under vacuum to give an colorless oil, which was purified by column chromatography (petroleum ether/ethyl acetate, 1:1) to give compound 215 as a white solid (111 mg, 67%). MALDITOF-MS: Calcd for C₁₀₆H₁₂₂O₃₆: 1970.76 [M]⁺. Found 1993.85 [M+Na]⁺.

### Preparation of (216)

Compound 215 (110 mg, 0.055 mmol) was dissolved in anhyd CH₂Cl₂-MeOH (1:2, 18 mL), and then 1.0 M NaOMe in MeOH (0.2 mL) was added at 0° C. After stirring at room temperature for 5 h, TLC (n-BuOH-EtOH—H₂O, 2:1:0.5) indicated that the reaction was complete. The solution was neutralized with ion-exchange resin (H⁺), and then filtered and concentrated. The residue was purified by Bio-gel P₂ column to afford 216 as an amorphous solid (54 mg, 96%): Selected ¹H NMR (400 MHz, C₆D₅N): δ5.27 (d, 1 J 7.5 Hz, H-1), 5.24 (d, 1H, J 6.9 Hz, H-1), 5.14 (d, 1H, J 7.5 Hz, H-1), 5.06 (d, 1H, J 7.2 Hz, H-1). ¹³C NMR (100 MHz, CD₅N): δ 103.6, 103.3, 103.0, 102.4. MALDITOF-MS: Calcd for C₅₀H₉₄O₂₃: 1052.54 [M]⁺. Found 1075.70 [M+Na]⁺.

The foregoing broadly defines the present invention without limitation. Variations and modifications as will be readily apparent to those skilled in this art are intended to be included within the present invention as defined in the appended claims.

1. A method of preparing a compound of general formula I: 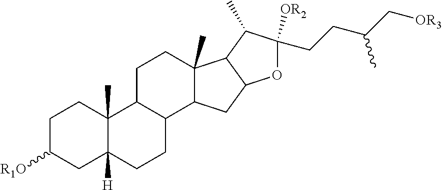
wherein, independently of each other,
R₁ represents hydrogen or an ester, ether or sugar residue;
R₂ represents hydrogen; and
R₃ represents hydrogen or a sugar residue;
or a protected form thereof in which any one or more of the groups R₁ and R₃ are, independently from each other, protected by a removable protecting group to prevent an undesirable reaction of the group;
which comprises selectively reducing a diketone compound of general formula II: 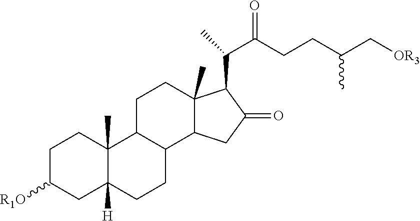
wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group to prevent an undesirable reaction of the residue;
using a borohydride reducing agent in a suitable solvent.

2. A method according to claim 1, wherein the compound of general formula II is a compound of general formula IIa: 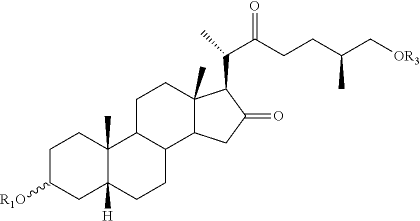
wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group to prevent an undesirable reaction of the residue.

3. A method according to claim 1, wherein the compound of general formula II is a compound of general formula IIb: 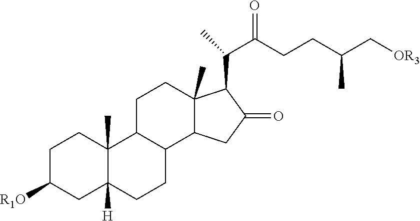
wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group to prevent an undesirable reaction of the residue.

4. A method according to claim 1, for preparing timosaponin BII or a prodrug form thereof.

5. A method according to claim 1, wherein the reducing agent is an unhindered borohydride reducing agent.

6. A method according to claim 1, wherein the reducing agent is sodium borohydride.

7. A method according to claim 1, wherein the solvent for the selective reduction of the diketone of general formula II is a mixture of a polar organic solvent and a non-polar organic solvent which is miscible with the polar solvent.

8. A method according to claim 7, wherein the solvent is an about 2:1 to about 20:1 by volume mixture of 2-propanol and dichloromethane.

9. A method according to claim 1, wherein, prior to the reduction, the compound of general formula II is subjected to one or more coupling reactions to add optionally protected ester, ether and/or sugar moieties to one or both of the residues R₁ and R₃.

10. A method according to claim 9, wherein the compound of general formula II is subjected to one or more coupling reaction to add one or more optionally protected sugar moieties to one or more sugar moieties of one or both of the residues R₁ and R₃.

11. A method according to claim 9, wherein the sugar coupling reaction uses a sugar trihaloacetimidate as an activated sugar moiety for coupling.

12. A method according to claim 9, wherein the sugar coupling reaction uses a thioglycoside as an activated sugar moiety for coupling.

13. A method according to any claim 9, wherein cyclic sugars are coupled successively to assemble a polysaccharide in situ on the compound of formula II at one or more of the residues R₁ and R₃.

14. Compounds of general formula I: 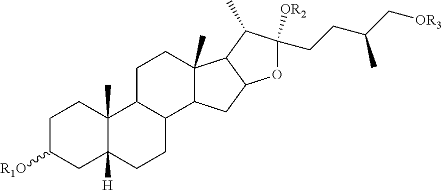
wherein, independently of each other,
R₁ denotes H, COCH₃, CO(CH₂)ₙCH₃ (n=1-6), CₘH₂ₘ₊₁ (m=1-6), Gal, Glc, β-D-Glc-(1→2)-β-D-Gal, β-D-Glc-(1→4)-β-D-Gal, β-D-Glc-(1→2)-β-D-Glc, β-D-Glc-(1→4)-β-D-Glc, β-D-Glc-(1→6)-β-D-Glc, α-L-Rha-(1→2)-β-D-Glc, α-L-Rha-(1→4)-β-D-Glc, α-L-Rha-(1→6)-β-D-Gal, α-L-Rha-(1→4)-[α-L-Rha-(1→2)]-β-D-Glc, β-D-Glc-(1→4)-[α-L-Rha-(1→2)]-β-D-Glc, β-D-Glc-(1→2)-[α-L-Rha-(1→4)]-β-D-Glc, β-D-Glc-(1→4)-[α-L-Rha-(1→2)]-β-D-Gal, α-L-Rha-(1→4)-[β-DGlc-(1→2)]-β-D-Glc, β-D-Glc-(1→4)-β-D-Glc-(1→4)-β-D-Gal, β-D-Glc-(1→4)-β-D-Glc-(1→2)-β-D-Gal, β-D-Glc-(1→4)-β-D-Glc-(1→4)-β-D-Glc or β-D-Glc-(1→4)-β-D-Glc-(1→2)-β-D-Glc;
R₂ denotes H or CₘH₂ₘ₊₁ (m=1-6); and
R₃ denotes H, α-L-Fuc, β-D-Xyl, β-D-Ara, α-L-Rha, β-D-Gal and β-D-Glc;
excluding the compounds disclosed in WO-A-99/16786, WO-A-2005/105108 and WO-A-2005/105824 and the publications referred to therein.

15. Compounds according to claim 14, when prepared using the method according to claim 1.

16. Compounds of general formula II: 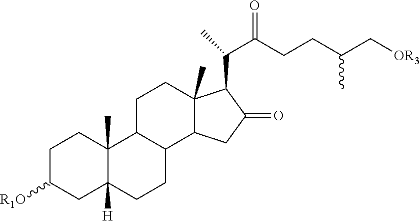
wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group.

17. Compounds according to claim 16, being compounds of general formula IIa: 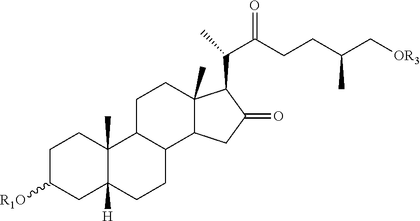
wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group.

18. Compounds according to claim 16, being compounds of general formula IIb: 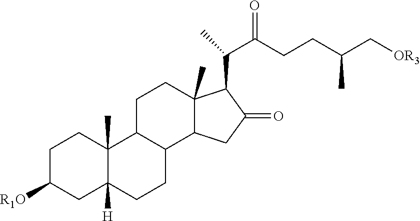
wherein, independently of each other,
R₁ represents hydrogen or an ester, ether or sugar residue; and
R₃ represents hydrogen or a sugar residue;
or a protected form thereof in which any one or both of the groups R₁ and R₃ are, independently from each other, protected by a removable protecting group.

19. Compounds according to claim 18, wherein, independently of each other,
R₁ denotes H, COCH₃, CO(CH₂)ₙCH₃ (n=1-6), CₘH₂ₘ₊₁ (m=1-6), Gal, Glc, β-D-Glc-(1→2)-β-D-Gal, β-D-Glc-(1→4)-β-D-Gal, β-D-Glc-(1→2)-β-D-Glc, β-D-Glc-(1→4)-β-D-Glc, β-D-Glc-(1→6)-β-D-Glc, α-L-Rha-(1→2)-β-D-Glc, α-L-Rha-(1→4)-β-D-Glc, α-L-Rha-(1→6)-β-D-Gal, α-L-Rha-(1→4)-[α-L-Rha-(1→2)]-β-D-Glc, β-D-Glc-(1→4)-[α-L-Rha-(1→2)]-β-D-Glc, β-D-Glc-(1→2)-[α-L-Rha-(1→4)]-β-D-Glc, β-D-Glc-(1→4)-[α-L-Rha-(1→2)]-β-D-Gal, α-L-Rha-(1→4)-[β-DGlc-(1→2)]-β-D-Glc, β-D-Glc-(1→4)-β-D-Glc-(1→4)-β-D-Gal, β-D-Glc-(1→4)-β-D-Glc-(1→2)-β-D-Gal, β-D-Glc-(1→4)-β-D-Glc-(1→4)-β-D-Glc or β-D-Glc-(1→4)-β-D-Glc-(1→2)-β-D-Glc; and
R₃ denotes H, α-L-Fuc, β-D-Ara, α-L-Rha, β-D-Gal and β-D-Glc.
